(19) United States
(12) Patent Application Publication
(10) Pub. No.: US 2024/0318180 A1
(43) Pub. Date: Sep. 26, 2024

(54) ANTISENSE OLIGONUCLEOTIDES TARGETING ACTL6B

(71) Applicant: Hoffmann-La Roche Inc., Little Falls, NJ (US)

(72) Inventors: Lars JOENSON, Viby Sjaelland (DK); Bettina NORDBO, Hørsholm (DK); Jonas VIKESAA, Fredensborg (DK)

(21) Appl. No.: 18/735,668

(22) Filed: Jun. 6, 2024

Related U.S. Application Data
(63) Continuation of application No. PCT/EP2022/084365, filed on Dec. 5, 2022.

(30) Foreign Application Priority Data
Dec. 7, 2021 (EP) 21212904.3

(51) Int. Cl. C12N 15/113

(52) U.S. Cl. CPC C12N 15/113; C12N 2310/11; C12N 2310/315; C12N 2310/321; C12N 2310/3231; C12N 2310/3233; C12N 2320/33

(57) ABSTRACT

The present invention relates to antisense oligonucleotide splice modulators of actin-like 6B (ACTL6B). These antisense oligonucleotide splice modulators are complementary, such as fully complementary, to the ACTL6B precursor-mRNA, and are capable of increasing or restoring expression of ACTL6B in TDP-43 depleted cells, such as for use in conditions and medical indications where TDP-43 is functionally depleted.

The present invention relates to antisense oligonucleotide splice modulators of actin-like 6B (ACTL6B). These antisense oligonucleotide splice modulators are complementary, such as fully complementary, to the ACTL6B precursor-mRNA, and are capable of increasing or restoring expression of ACTL6B in TDP-43 depleted cells, such as for use in conditions and medical indications where TDP-43 is functionally depleted.

### SEQUENCE LISTING

The instant application contains a Sequence Listing which has been submitted electronically in XML format and is hereby incorporated by reference in its entirety. Said XML copy, created on Jun. 5, 2024, is named 51551-022001_Sequence_Listing_6_5_24 and is 285,479 bytes in size.

### BACKGROUND

TAR DNA binding protein 43 (TDP-43) is a versatile RNA/DNA binding protein involved in RNA-related metabolism. Dysregulation of TDP-43 deposits act as inclusion bodies in the brain and spinal cord of patients with the motor neuron diseases: amyotrophic lateral sclerosis (ALS) and frontotemporal lobar degeneration (FTLD) (Prasad et al., Front. Mol. Neurosci., 2019).

TDP-43 depletion is indicated in a range of diseases, referred to as TDP-43 pathologies, and including for example diseases such as amyotrophic lateral sclerosis (ALS), frontotemporal lobar degeneration (FTLD), Progressive supranuclear palsy (PSP), Primary lateral sclerosis, Progressive muscular atrophy, Alzheimer's disease, Parkinson's disease, autism, Hippocampal sclerosis dementia, Down syndrome, Huntington's disease, polyglutamine diseases, such as spinocerebellar ataxia 3, myopathies and Chronic Traumatic Encephalopathy.

ACTL6B is involved in chromatin remodelling during neuronal differentiation. It is a splice variant of ACTL6A, and replaces ACTL6A once a stem cell has finished differentiating into a mature neuron.

We show that splicing of ACTL6B is controlled, at least in part, by TDP-43 due a to a TDP-43 binding site present within the ACTL6B pre-mRNA sequence.

### SUMMARY OF INVENTION

The present inventors have surprisingly determined that ACTL6B mRNA splicing changes if TDP-43 is depleted in a cell.

The inventors therefore hypothesised that modifying ACTL6B splicing patterns, may be able to ameliorate the detrimental effects of TDP-43 depletion on neuronal cells.

Here the inventors have used antisense oligonucleotide ACTL6B splice modulators to increase expression of ACTL6B.

In one aspect the invention provides an antisense oligonucleotide actin-like 6B (ACTL6B) splice modulator, wherein said antisense oligonucleotide splice modulator is 8 to 40 nucleotides in length and comprises a contiguous nucleotide sequence of at least 8 nucleotides in length which is complementary to the ACTL6B precursor-mRNA.

In some embodiments, the antisense oligonucleotide splice modulator may be capable of increasing the expression of actin-like 6B (ACTL6B) in a TDP-43 depleted cell.

In some embodiments, the antisense oligonucleotide splice modulator may be capable of decreasing expression of a ACTL6B mutant polypeptide, such as a splice variant of ACTL6B, in a TDP-43 depleted cell.

The inventors have surprisingly determined that in TDP-43 depleted cells, increased expression of an ACTL6B splice variant including an additional exon is observed. This leads to a decrease in production of the functionally active wild-type (WT) ACTL6B polypeptide. In some embodiments the splice variant may therefore comprise a polypeptide sequence encoded by an additional exon, when compared to the wild-type ACTL6B polypeptide sequence.

In some embodiments, the mutant ACTL6B splice variant may comprise an insertion, such as an insertion of about 23 amino acids, when compared to the wild-type ACTL6B polypeptide sequence.

In some embodiments the contiguous nucleotide sequence of the antisense oligonucleotide splice modulator may be complementary to a splice enhancer site in the ACTL6B precursor-mRNA.

In some embodiments the contiguous nucleotide sequence of the antisense oligonucleotide splice modulator may be complementary to a sequence selected from SEQ ID NOs: 199 to 205.

In some embodiments, the contiguous nucleotide sequence of the antisense oligonucleotide splice modulator may be complementary to a sequence selected from SEQ ID NOs: 6-97 and 190-193.

In some embodiments the contiguous nucleotide sequence of the antisense oligonucleotide splice modulator may be complementary to a sequence selected from SEQ ID NOs: 26, 28, 29, 30, 31, 32, 33, 38, 39, 46, 47, 48, 52, 53, 55 and 72.

In some embodiments the contiguous nucleotide sequence of the antisense oligonucleotide splice modulator may be complementary to a sequence selected from SEQ ID NOs: 28, 29, 30, 31, 32, 33 and 47.

In some embodiments, the contiguous nucleotide sequence of the antisense oligonucleotide splice modulator may be a sequence selected from SEQ ID Nos 98-189 and 194-197, or at least 10 contiguous nucleotides thereof.

In some embodiments, the contiguous nucleotide sequence of the antisense oligonucleotide splice modulator may be a sequence selected from the group consisting of SEQ ID NOs: 118, 120, 121, 122, 123, 124, 125, 130, 131, 138, 139, 140, 144, 145, 147 and 164, or at least 10 contiguous nucleotides thereof.

In some embodiments, the contiguous nucleotide sequence of the antisense oligonucleotide splice modulator may be a sequence selected from the group consisting of SEQ ID NOs: 120, 121, 122, 123, 124, 125 and 139, or at least 10 contiguous nucleotides thereof.

In some embodiments, the antisense oligonucleotide splice modulator may be at least 12 nucleotides in length, such as at least 13, 14, 15, 16, 17, 18, 19, 20, 21, 22, 23, 24, 25, 26, 27, 28, 29, 30, 31 or 32 nucleotides in length.

In some embodiments, the contiguous nucleotide sequence of the antisense oligonucleotide splice modulator may be the same length as the antisense oligonucleotide splice modulator.

In some embodiments, the antisense oligonucleotide splice modulator may comprise one or more modified nucleosides, such as a 2′ sugar modified nucleoside, which may be independently selected from the group consisting of 2′-O-alkyl-RNA; 2′-O-methyl RNA (2′-OMe); 2′-alkoxy-RNA; 2′-O-methoxyethyl-RNA (2′-MOE); 2′-amino-DNA; 2′-fluro-RNA; 2′-fluoro-DNA; arabino nucleic acid (ANA); 2′-fluoro-ANA; locked nucleic acid (LNA), or any combination thereof.

In some embodiments, the contiguous nucleotide sequence of the antisense oligonucleotide splice modulator may comprise 2′-O-methoxyethyl-RNA (2′-MOE) nucleosides, optionally linked by phosphorothioate internucleoside linkages.

In some embodiments, one or more of the modified nucleosides may be a locked nucleic acid nucleoside (LNA), such as an LNA nucleoside selected from the group consisting of constrained ethyl nucleoside (cEt), and β-D-oxy-LNA.

In some embodiments, the contiguous nucleotide sequence of the antisense oligonucleotide splice modulator may be at least 75%, such as at least 80%, at least 85%, at least 90% or at least 95%, complementary to the ACTL6B precursor-mRNA sequence.

In other embodiments, the contiguous nucleotide sequence contiguous nucleotide sequence of the may be fully complementary to the ACTL6B precursor-mRNA.

In some embodiments, the antisense oligonucleotide splice modulator may not comprise a region of more than 3, or more than 4, contiguous DNA nucleosides, and may not be capable of mediating RNAseH cleavage.

In some embodiments, one or more, or all, of the internucleoside linkages within the antisense oligonucleotide splice modulator may be modified. For examples, the modified internucleoside linkages may comprise a phosphorothioate linkage.

In some embodiments, the antisense oligonucleotide splice modulator may be covalently attached to at least one conjugate moiety.

In some embodiments, the antisense oligonucleotide splice modulator may be in the form of a pharmaceutically acceptable salt, such as a sodium salt or a potassium salt.

In another aspect there is provided a pharmaceutical composition comprising the antisense oligonucleotide splice modulator of the invention, and a pharmaceutically acceptable diluent, solvent, carrier, salt and/or adjuvant.

In another aspect there is provided a method, such as an in vivo or in vitro method, for increasing ACTL6B expression in a cell, said method comprising administering an antisense oligonucleotide splice modulator or pharmaceutical composition of the invention, in an effective amount to said cell, which may express aberrant or exhibits depleted levels of TDP-43.

In another aspect the invention provides a method for treating or preventing a disease in a subject comprising administering a therapeutically or prophylactically effective amount of an antisense oligonucleotide splice modulator or pharmaceutical composition of the invention to a subject suffering from or susceptible to the disease.

In another aspect there is provided an antisense oligonucleotide splice modulator or a pharmaceutical composition of the invention for use as a medicament.

In another aspect there is provided an antisense oligonucleotide splice modulator or pharmaceutical composition of the invention for use in the treatment or prevention of disease in a subject.

In another aspect there is provided an antisense oligonucleotide splice modulator or pharmaceutical composition of the invention for the preparation of a medicament for treatment or prevention of a disease in a subject.

In all aspects of the invention, the disease may be a neurological disorder selected from the group consisting of amyotrophic lateral sclerosis (ALS), frontotemporal lobar degeneration (FTLD), Progressive supranuclear palsy (PSP), Primary lateral sclerosis, Progressive muscular atrophy, Alzheimer's disease, Parkinsons disease, Autism, Hippocampal sclerosis dementia, Down syndrome, Huntington's disease, polyglutamine diseases, such as spinocerebellar ataxia 3, myopathies and Chronic Traumatic Encephalopathy.

In particular embodiments, the disease may be a neurological disorder selected from the group consisting of amyotrophic lateral sclerosis (ALS) and frontotemporal lobar degeneration (FTLD).

### BRIEF DESCRIPTION OF FIGURES

FIG. 1 displays a screenshot from the CLC Genomics Workbench software where the NGS read mapping from the ACTL6B gene can be seen. The inclusion of the new exon in the sample treated with compound A (SEQ ID 198) is shown with an arrow.

### DETAILED DESCRIPTION

The inventors have identified that the splicing of ACTL6B is affected by TDP-43. This is thought to lead to the production of non-functional, or less functional, ACTL6B in TDP-43 cells.

Without wishing to be bound by theory, it is considered that in TDP-43 depleted cells ACTL6B may be spliced such that an addition exon is included. This additional exon may be 23 amino acids in length. The inclusion of this additional exon may lead to formation of a polypeptide with is less functionally active than wild-type ACTL6B. Such an alternatively spliced polypeptide is referred to herein as a “mutant ACTL6B polypeptide”, a “splicing variant of ACTL6B” or an “ACTL6B splice variant”.

The inventors have also determined that production of an ACTL6B splicing variant can be reduced using an antisense oligonucleotide splice modulator. Herein an antisense oligonucleotide splice modulator of the invention may also be referred to as an oligonucleotide of the invention or an antisense oligonucleotide of the invention.

The oligonucleotide splice modulators of the invention may target a splice enhancer site in the ACTL6B precursor-mRNA. This may reduce alternative splicing, thereby increasing conventional splicing and the production of wild-type ACTL6B protein.

Enhanced wild-type ACTL6B expression is desirable to treat a range of disorders which are characterised by, or caused by, reduced expression of ACTL6B. These include amyotrophic lateral sclerosis (ALS), frontotemporal lobar degeneration (FTLD), Progressive supranuclear palsy (PSP), Primary lateral sclerosis, Progressive muscular atrophy, Alzheimer's disease, Parkinsons disease, Autism, Hippocampal sclerosis dementia, Down syndrome, Huntington's disease, polyglutamine diseases, such as spinocerebellar ataxia 3, myopathies and Chronic Traumatic Encephalopathy.

### Splice Modulation

The antisense oligonucleotides of the invention are ACTL6B splice modulators, that is they affect the splicing of ACTL6B pre-mRNA. Herein the oligonucleotides of the invention may be referred to as “antisense oligonucleotide splice modulators”.

In some embodiments, the antisense oligonucleotide splice modulators of the invention may be complementary to the ACTL6B precursor-mRNA.

In some embodiments, the ACTL6B precursor-mRNA may have the sequence of SEQ ID NO 1. SEQ ID NO 1 is provided herein as a reference sequence and it will be understood that the target precursor-mRNA may be an allelic variant of SEQ ID NO 1, such as an allelic variant which comprises one or more polymorphisms.

In some embodiments, the antisense oligonucleotide splice modulator may be capable of increasing the expression of ACTL6B in a TDP-43 depleted cell. Herein, it is anticipated that expression of wild-type, i.e. conventionally spliced, ACTL6B which will be increased by exposure to the antisense oligonucleotide splice modulator of the invention.

Without wishing to be bound by theory, it is thought that the antisense oligonucleotide splice modulators of the invention may increase conventional splicing of ACTL6B precursor-mRNA. This is thought to lead to an increase in the amount of conventionally spliced mature ACTL6B mRNA, which in turn is thought to lead to an increase in the amount of wild-type ACTL6B protein.

Herein the terms “wild-type” and “conventionally spliced” will be used interchangeably.

In some embodiments the wild-type (i.e. conventionally spliced) mature ACTL6B mRNA sequence may have the sequence of SEQ ID NO: 2, or a fragment or variant thereof. SEQ ID NO 2 is provided herein as a reference sequence and it will be understood that the conventionally spliced ACTL6B mRNA may be an allelic variant of SEQ ID NO 2, such as an allelic variant which comprises one or more polymorphisms.

In some embodiments, the wild-type ACTL6B protein may have the sequence of SEQ ID NO: 3, or a fragment or variant thereof. SEQ ID NO 3 is provided herein as a reference sequence and it will be understood that the wild-type ACTL6B protein may be an allelic variant of SEQ ID NO 3, such as an allelic variant which comprises one or more polymorphisms.

Herein, the term “increasing the expression of wild-type ACTL6B” is understood to mean increasing conventionally spliced ACTL6B mRNA levels, increasing wild-type ACTL6B protein levels or increasing conventionally spliced ACTL6B mRNA levels and wild-type ACTL6B protein levels.

In certain embodiments, the antisense oligonucleotide splice modulators of the present invention may increase conventional splicing of ACTL6B precursor-mRNA by at least about 10% compared to a control. More preferably the antisense oligonucleotide splice modulators of the present invention may increase conventional splicing of ACTL6B precursor-mRNA by at least about 15%, at least about 20%, at least about 25%, at least about 30%, at least about 35%, at least about 40%, at least about 45%, at least about 50%, at least about 60%, at least about 70%, at least about 80%, at least about 90%, at least about 100%, at least about 200%, at least about 300%, at least about 400%, at least about 500%, at least about 600%, or more compared to a control.

In certain embodiments, the antisense oligonucleotide splice modulators of the present invention may increase the amount of wild-type ACTL6B protein by at least about 10% compared to a control. More preferably the antisense oligonucleotide splice modulators of the present invention may increase the amount of wild-type ACTL6B protein by at least about 15%, at least about 20%, at least about 25%, at least about 30%, at least about 35%, at least about 40%, at least about 45%, at least about 50%, at least about 60%, at least about 70%, at least about 80%, at least about 90%, at least about 100%, at least about 200%, at least about 300%, at least about 400%, at least about 500%, at least about 600%, or more compared to a control.

In certain embodiments, the antisense oligonucleotide splice modulators of the present invention may increase conventional splicing of ACTL6B precursor-mRNA and increase the amount of wild-type ACTL6B protein by at least about 10% compared to a control. More preferably the antisense oligonucleotide splice modulators of the present invention may increase conventional splicing of ACTL6B precursor-mRNA and increase the amount of wild-type ACTL6B protein by at least about 15%, at least about 20%, at least about 25%, at least about 30%, at least about 35%, at least about 40%, at least about 45%, at least about 50%, at least about 60%, at least about 70%, at least about 80%, at least about 90%, at least about 100%, at least about 200%, at least about 300%, at least about 400%, at least about 500%, at least about 600%, or more compared to a control.

Preferably, the antisense oligonucleotide splice modulators of the present invention increase the amount of wild-type ACTL6B by decreasing expression of a ACTL6B mutant polypeptide in a TDP-43 depleted cell.

The ACTL6B mutant polypeptide may be a splicing variant of ACTL6B. Herein, the term “splicing variant” or “splice variant” includes, but is not limited to, a variant mature mRNA which includes an additional exon relative to the wild-type ACTL6B mature mRNA sequence. The wild-type ACTL6B mature mRNA sequence may be SEQ IS NO 2.

In some embodiments the inclusion of an additional exon within the ACTL6B mature mRNA sequence may lead to an insertion in the translated polypeptide sequence, relative to the wild-type ACTL6B polypeptide sequence. The wild-type ACTL6B polypeptide may have the sequence of SEQ ID NO 3.

In some embodiments the insertion may be about 23 amino acids, relative to the wild-type ACTL6B polypeptide sequence. In other embodiments the insertion may be 20, 21, 22, 23, 24, 25, 26, 27, 28, 29, 30 or more amino acids, relative to the wild-type ACTL6B polypeptide sequence. The wild-type ACTL6B polypeptide may have the sequence of SEQ ID NO 3.

In some embodiments, the mutant polypeptide may be encoded by the nucleotide sequence of SEQ ID NO: 4, or a fragment or variant thereof. SEQ ID NO 4 is provided herein as a reference sequence and it will be understood that the nucleic acid sequence encoding the mutant ACTL6B polypeptide may be an allelic variant of SEQ ID NO 4, such as an allelic variant which comprises one or more polymorphisms.

In other embodiments, the ACTL6B mutant polypeptide may have the sequence of SEQ ID NO: 5, or a fragment or variant thereof. SEQ ID NO 5 is provided herein as a reference sequence and it will be understood that the mutant ACTL6B polypeptide may be an allelic variant of SEQ ID NO 5, such as an allelic variant which comprises one or more polymorphisms.

Herein, the term “decreasing expression of a ACTL6B mutant” is understood to mean decreasing alternatively spliced ACTL6B mature mRNA levels, decreasing mutant ACTL6B polypeptide levels, or decreasing alternatively spliced ACTL6B mature mRNA levels and decreasing mutant ACTL6B polypeptide levels.

In some embodiments the antisense oligonucleotide splice modulators of the invention are capable of decreasing the level of alternatively spliced ACTL6B mature mRNA by at least 10% compared to a control. More preferably the antisense oligonucleotide splice modulators of the invention are capable of decreasing the level of alternatively spliced ACTL6B mature mRNA by at least about 15%, at least about 20%, at least about 25%, at least about 30%, at least about 35%, at least about 40%, at least about 45%, at least about 50%, at least about 60%, at least about 70%, at least about 80%, at least about 90%, at least about 100%, at least about 200%, at least about 300%, at least about 400%, at least about 500%, at least about 600%, or more compared to a control.

In some embodiments the antisense oligonucleotide splice modulators of the invention are capable of decreasing mutant ACTL6B polypeptide levels by at least 10% compared to a control. More preferably the antisense oligonucleotide splice modulators of the invention are capable of decreasing mutant ACTL6B polypeptide levels by at least about 15%, at least about 20%, at least about 25%, at least about 30%, at least about 35%, at least about 40%, at least about 45%, at least about 50%, at least about 60%, at least about 70%, at least about 80%, at least about 90%, at least about 100%, at least about 200%, at least about 300%, at least about 400%, at least about 500%, at least about 600%, or more compared to a control.

In some embodiments the antisense oligonucleotide splice modulators of the invention are capable of decreasing alternatively spliced ACTL6B mature mRNA levels and decreasing wild-type ACTL6B polypeptide levels by at least 10% compared to a control. More preferably the antisense oligonucleotide splice modulators of the invention are capable of decreasing alternatively spliced mature ACTL6B mRNA levels and decreasing mutant ACTL6B polypeptide levels by at least about 15%, at least about 20%, at least about 25%, at least about 30%, at least about 35%, at least about 40%, at least about 45%, at least about 50%, at least about 60%, at least about 70%, at least about 80%, at least about 90%, at least about 100%, at least about 200%, at least about 300%, at least about 400%, at least about 500%, at least about 600%, or more compared to a control.

### Control

By the term “control”, when used in relation to measurements of the effect of an antisense oligonucleotide splice modulator, it is generally understood that the control is a cell that has not been exposed to the antisense oligonucleotide splice modulator of the invention.

Alternatively, an increase in the expression of wild-type ACTL6B or a decrease in the expression of a ACTL6B mutant may be determined by reference to the amount of wild-type and/or mutant ACTL6B mRNA and/or polypeptide expressed before exposure to the antisense oligonucleotide splice modulator of the invention.

In other embodiments, the control may be a cell treated with a non-targeting oligonucleotide.

In some embodiments, the control may be a mock transfection, for example wherein cells are treated with PBS.

### Oligonucleotides

The term “oligonucleotide” as used herein is defined as it is generally understood by the skilled person as a molecule comprising two or more covalently linked nucleosides. Such covalently bound nucleosides may also be referred to as nucleic acid molecules or oligomers.

Oligonucleotides are commonly made in the laboratory by solid-phase chemical synthesis followed by purification and isolation. When referring to a sequence of the oligonucleotide, reference is made to the sequence or order of nucleobase moieties, or modifications thereof, of the covalently linked nucleotides or nucleosides. The antisense oligonucleotide splice modulators of the invention are man-made, and are chemically synthesised, and are typically purified or isolated. The antisense oligonucleotide splice modulators of the invention may comprise one or more modified nucleosides such as 2′ sugar modified nucleosides. The antisense oligonucleotide splice modulators of the invention may comprise one or more modified internucleoside linkages, such as one or more phosphorothioate internucleoside linkages.

In some embodiments, the antisense oligonucleotide splice modulators of the invention are single stranded oligonucleotides.

In some embodiments, the antisense oligonucleotide splice modulators of the invention are 8 to 40 nucleotides in length.

In some embodiments, the antisense oligonucleotide splice modulators of the invention are 8 to 40 nucleotides in length and comprise a contiguous nucleotide sequence of 8 to 40 nucleotides.

In some embodiments, the antisense oligonucleotide splice modulators of the invention are 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 nucleotides in length.

In some embodiments the antisense oligonucleotide splice modulators of the invention are at least 12 nucleotides in length.

In some embodiments the antisense oligonucleotide splice modulators of the invention are at least 14 nucleotides in length.

In some embodiments the antisense oligonucleotide splice modulators of the invention are at least 16 nucleotides in length.

In some embodiments the antisense oligonucleotide splice modulators of the invention are at least 18 nucleotides in length.

Preferably, the antisense oligonucleotide splice modulators of the invention are 16 to 20 nucleotides in length.

More preferably, the antisense oligonucleotide splice modulators of the invention are 18 to 20 nucleotides in length.

### Contiguous Nucleotide Sequence

The term “contiguous nucleotide sequence” as used herein refers to the region of the antisense oligonucleotide splice modulator of the invention which is complementary to a target nucleic acid, which may be or may comprise an oligonucleotide motif sequence. The term is used interchangeably herein with the term “contiguous nucleobase sequence”.

The antisense oligonucleotide splice modulator comprises the contiguous nucleotide sequence, and may optionally comprise further nucleotide(s), for example a nucleotide linker region which may be used to attach a functional group (e.g. a conjugate group) to the contiguous nucleotide sequence. The nucleotide linker region may or may not be complementary to the target nucleic acid.

It is understood that the contiguous nucleotide sequence of the antisense oligonucleotide splice modulator cannot be longer than the antisense oligonucleotide splice modulator as such and that the antisense oligonucleotide splice modulator cannot be shorter than the contiguous nucleotide sequence.

In some embodiments, the entire nucleotide sequence of the antisense oligonucleotide splice modulator of the invention is the contiguous nucleotide sequence.

The contiguous nucleotide sequence is the sequence of nucleotides in the antisense oligonucleotide splice modulator of the invention which are complementary to, and in some instances fully complementary to, the target nucleic acid, target sequence, or target site sequence.

In some embodiments, the contiguous nucleotide sequence is 8 to 40 nucleotides in length.

In some embodiments, the contiguous nucleotide sequence is 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 nucleotides in length.

In some embodiments the contiguous nucleotide sequence is at least 12 nucleotides in length.

In some embodiments the contiguous nucleotide sequence is at least 14 nucleotides in length.

In some embodiments the contiguous nucleotide sequence is at least 16 nucleotides in length.

In some embodiments the contiguous nucleotide sequence is at least 18 nucleotides in length.

In a preferred embodiment the contiguous nucleotide sequence is 16 to 20 nucleotides in length.

More preferably, the contiguous nucleotide sequence is 18 to 20 nucleotides in length.

In some embodiments the antisense oligonucleotide splice modulator of the invention consists of the contiguous nucleotide sequence.

In some embodiments the antisense oligonucleotide splice modulator of the invention is the contiguous nucleotide sequence.

### Antisense Oligonucleotide Splice Modulator Targeting ACTL6B Precursor-mRNA

The antisense oligonucleotide splice modulators of the invention comprise a contiguous nucleotide sequence which is complementary to the ACTL6B precursor-mRNA.

The ACTL6B precursor-mRNA may be described as the target for the contiguous nucleotide sequence or for the antisense oligonucleotide splice modulator. Put another way, the antisense oligonucleotide splice modulator targets the ACTL6B precursor-mRNA.

In some embodiments the target sequence may have the sequence of SEQ ID NO 1. SEQ ID NO 1 is provided herein as a reference sequence and it will be understood that the ACTL6B precursor-mRNA sequence may be an allelic variant of SEQ ID NO 1, such as an allelic variant which comprises one or more polymorphisms. This applies equally to all sequences identified as target sequences herein.

In one aspect, the invention relates to an antisense oligonucleotide splice modulator wherein said antisense oligonucleotide splice modulator is 8 to 40 nucleotides in length and comprises a contiguous nucleotide sequence of at least 8 nucleotides in length which is complementary to SEQ ID NO 1.

In some embodiments, the antisense oligonucleotide splice modulator of the invention comprises a contiguous sequence which is at least about 75% complementary, at least about 80% complementary, at least about 85% complementary, at least about 90% complementary, at least about 95% complementary, or fully complementary (i.e. 100% complementary) to SEQ ID NO 1. Here's complementarity is determined across the length of the contiguous nucleotide sequence.

In some embodiments the antisense oligonucleotide splice modulator of the invention comprises a contiguous sequence which is at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% complementary or fully complementary (i.e. 100% complementary) to SEQ ID NO 1.

In some embodiments, the antisense oligonucleotide splice modulator of the invention comprises a contiguous sequence which may comprise one, two or three mismatches between the contiguous nucleotide sequence and the target nucleic acid.

In a preferred embodiment the oligonucleotide of the invention, or contiguous nucleotide sequence thereof is fully complementary (100% complementary) to SEQ ID NO 1, across the length of the contiguous nucleotide sequence.

In one embodiment the contiguous nucleotide sequence is complementary to a splice enhancer site in the ACTL6B precursor-mRNA.

An aspect of the present invention relates to an antisense oligonucleotide splice modulator, which comprises a contiguous nucleotide sequence of 8 to 40 nucleotides in length which is complementarity to SEQ ID NO 199.

In some embodiments, the antisense oligonucleotide splice modulator of the invention comprises a contiguous sequence which is at least 90% complementary, such as at least 91%, such as at least 92%, such as at least 93%, such as at least 94%, such as at least 95%, such as at least 96%, such as at least 97%, such as at least 98%, or 100% complementary to SEQ ID NO 199.

In a preferred embodiment the antisense oligonucleotide splice modulator of the invention comprises a contiguous sequence which is fully complementary (i.e. 100% complementary) to SEQ ID NO 199.

An aspect of the present invention relates to an antisense oligonucleotide splice modulator, which comprises a contiguous nucleotide sequence of 8 to 40 nucleotides in length which is complementary to SEQ ID NO 200.

In some embodiments, the antisense oligonucleotide splice modulator of the invention comprises a contiguous sequence which is at least 90% complementary, such as at least 91%, such as at least 92%, such as at least 93%, such as at least 94%, such as at least 95%, such as at least 96%, such as at least 97%, such as at least 98%, or 100% complementary to SEQ ID NO 200.

In a preferred embodiment the antisense oligonucleotide splice modulator of the invention comprises a contiguous sequence which is fully complementary (i.e. 100% complementary) to SEQ ID NO 200.

An aspect of the present invention relates to an antisense oligonucleotide splice modulator, which comprises a contiguous nucleotide sequence of 8 to 40 nucleotides in length which is complementary to SEQ ID NO 201.

In some embodiments, the antisense oligonucleotide splice modulator of the invention comprises a contiguous sequence which is at least 90% complementary, such as at least 91%, such as at least 92%, such as at least 93%, such as at least 94%, such as at least 95%, such as at least 96%, such as at least 97%, such as at least 98%, or 100% complementary to SEQ ID NO 201.

In a preferred embodiment the antisense oligonucleotide splice modulator of the invention comprises a contiguous sequence which is fully complementary (i.e. 100% complementary) to SEQ ID NO 201.

An aspect of the present invention relates to an antisense oligonucleotide splice modulator, which comprises a contiguous nucleotide sequence of 8 to 40 nucleotides in length which is complementary to SEQ ID NO 202.

In some embodiments, the antisense oligonucleotide splice modulator of the invention comprises a contiguous sequence which is at least 90% complementary, such as at least 91%, such as at least 92%, such as at least 93%, such as at least 94%, such as at least 95%, such as at least 96%, such as at least 97%, such as at least 98%, or 100% complementary to SEQ ID NO 202.

In a preferred embodiment the antisense oligonucleotide splice modulator of the invention comprises a contiguous sequence which is fully complementary (i.e. 100% complementary) to SEQ ID NO 202.

An aspect of the present invention relates to an antisense oligonucleotide splice modulator, which comprises a contiguous nucleotide sequence of 8 to 40 nucleotides in length which is complementary to SEQ ID NO 203.

In some embodiments, the antisense oligonucleotide splice modulator of the invention comprises a contiguous sequence which is at least 90% complementary, such as at least 91%, such as at least 92%, such as at least 93%, such as at least 94%, such as at least 95%, such as at least 96%, such as at least 97%, such as at least 98%, or 100% complementary to SEQ ID NO 203.

In a preferred embodiment the antisense oligonucleotide splice modulator of the invention comprises a contiguous sequence which is fully complementary (i.e. 100% complementary) to SEQ ID NO 203.

An aspect of the present invention relates to an antisense oligonucleotide splice modulator, which comprises a contiguous nucleotide sequence of 8 to 40 nucleotides in length which is complementary to SEQ ID NO 204.

In some embodiments, the antisense oligonucleotide splice modulator of the invention comprises a contiguous sequence which is at least 90% complementary, such as at least 91%, such as at least 92%, such as at least 93%, such as at least 94%, such as at least 95%, such as at least 96%, such as at least 97%, such as at least 98%, or 100% complementary to SEQ ID NO 204.

In a preferred embodiment the antisense oligonucleotide splice modulator of the invention comprises a contiguous sequence which is fully complementary (i.e. 100% complementary) to SEQ ID NO 204.

An aspect of the present invention relates to an antisense oligonucleotide splice modulator, which comprises a contiguous nucleotide sequence of 8 to 40 nucleotides in length which is complementary to SEQ ID NO 205.

In some embodiments, the antisense oligonucleotide splice modulator of the invention comprises a contiguous sequence which is at least 90% complementary, such as at least 91%, such as at least 92%, such as at least 93%, such as at least 94%, such as at least 95%, such as at least 96%, such as at least 97%, such as at least 98%, or 100% complementary to SEQ ID NO 205.

In a preferred embodiment the antisense oligonucleotide splice modulator of the invention comprises a contiguous sequence which is fully complementary (i.e. 100% complementary) to SEQ ID NO 205.

In one embodiment the target sequence is SEQ ID NO 199. Put another way, in some embodiments the contiguous nucleic acid is complementary to SEQ ID NO 199.

In one embodiment the target sequence is SEQ ID NO 200. Put another way, in some embodiments the contiguous nucleic acid is complementary to SEQ ID NO 200.

In one embodiment the target sequence is SEQ ID NO 201. Put another way, in some embodiments the contiguous nucleic acid is complementary to SEQ ID NO 201.

In one embodiment the target sequence is SEQ ID NO 202. Put another way, in some embodiments the contiguous nucleic acid is complementary to SEQ ID NO 202.

In one embodiment the target sequence is SEQ ID NO 203. Put another way, in some embodiments the contiguous nucleic acid is complementary to SEQ ID NO 203.

In one embodiment the target sequence is SEQ ID NO 204. Put another way, in some embodiments the contiguous nucleic acid is complementary to SEQ ID NO 204.

In one embodiment the target sequence is SEQ ID NO 205. Put another way, in some embodiments the contiguous nucleic acid is complementary to SEQ ID NO 205.

In some embodiments, the antisense oligonucleotide splice modulator of the invention comprises a contiguous nucleotide sequence of 8 to 40 nucleotides in length with at least 75% complementary, such as at least 80%, at least 85%, at least 90% or at least 95% or 100% complementarity, to a target nucleic acid region selected from the group consisting of SEQ ID NO 6, SEQ ID NO 7, SEQ ID NO 8, SEQ ID NO 9, SEQ ID NO 10, SEQ ID NO 11, SEQ ID NO 12, SEQ ID NO 13, SEQ ID NO 14, SEQ ID NO 15, SEQ ID NO 16, SEQ ID NO 17, SEQ ID NO 18, SEQ ID NO 19, SEQ ID NO 20, SEQ ID NO 21, SEQ ID NO 22, SEQ ID NO 23, SEQ ID NO 24, SEQ ID NO 25, SEQ ID NO 26, SEQ ID NO 27, SEQ ID NO 28, SEQ ID NO 29, SEQ ID NO 30, SEQ ID NO 31, SEQ ID NO 32, SEQ ID NO 33, SEQ ID NO 34, SEQ ID NO 35, SEQ ID NO 36, SEQ ID NO 37, SEQ ID NO 38, SEQ ID NO 39, SEQ ID NO 40, SEQ ID NO 41, SEQ ID NO 42, SEQ ID NO 43, SEQ ID NO 44, SEQ ID NO 45, SEQ ID NO 46, SEQ ID NO 47, SEQ ID NO 48, SEQ ID NO 49, SEQ ID NO 50, SEQ ID NO 51, SEQ ID NO 52, SEQ ID NO 53, SEQ ID NO 54, SEQ ID NO 55, SEQ ID NO 56, SEQ ID NO 57, SEQ ID NO 58, SEQ ID NO 59, SEQ ID NO 60, SEQ ID NO 61, SEQ ID NO 62, SEQ ID NO 63, SEQ ID NO 64, SEQ ID NO 65, SEQ ID NO 66, SEQ ID NO 67, SEQ ID NO 68, SEQ ID NO 69, SEQ ID NO 70, SEQ ID NO 71, SEQ ID NO 72, SEQ ID NO 73, SEQ ID NO 74, SEQ ID NO 75, SEQ ID NO 76, SEQ ID NO 77, SEQ ID NO 78, SEQ ID NO 79, SEQ ID NO 80, SEQ ID NO 81, SEQ ID NO 82, SEQ ID NO 83, SEQ ID NO 84, SEQ ID NO 85, SEQ ID NO 86, SEQ ID NO 87, SEQ ID NO 88, SEQ ID NO 89, SEQ ID NO 90, SEQ ID NO 91, SEQ ID NO 92, SEQ ID NO 93, SEQ ID NO 94, SEQ ID NO 95, SEQ ID NO 96, SEQ ID NO 97, SEQ ID NO 190, SEQ ID NO 191, SEQ ID NO 192 and SEQ ID NO 193.

In some embodiments the target sequence is selected from the group consisting of SEQ ID NO 26, SEQ ID NO 28, SEQ ID NO 29, SEQ ID NO 30, SEQ ID NO 31, SEQ ID NO 32, SEQ ID NO 33, SEQ ID NO 38, SEQ ID NO 39, SEQ ID NO 46, SEQ ID NO 47, SEQ ID NO 48, SEQ ID NO 52, SEQ ID NO 53, SEQ ID NO 55 and SEQ ID NO 72. Put another way, in some embodiments the contiguous nucleic acid is complementary to sequence is selected from the group consisting of SEQ ID NO 26, SEQ ID NO 28, SEQ ID NO 29, SEQ ID NO 30, SEQ ID NO 31, SEQ ID NO 32, SEQ ID NO 33, SEQ ID NO 38, SEQ ID NO 39, SEQ ID NO 46, SEQ ID NO 47, SEQ ID NO 48, SEQ ID NO 52, SEQ ID NO 53, SEQ ID NO 55 and SEQ ID NO 72.

In some embodiments the target sequence is selected from the group consisting of SEQ ID NO 28, SEQ ID NO 29, SEQ ID NO 30, SEQ ID NO 31, SEQ ID NO 32, SEQ ID NO 33 and SEQ ID NO 47. Put another way, in some embodiments the contiguous nucleic acid is complementary to sequence is selected from the group consisting of SEQ ID NO 28, SEQ ID NO 29, SEQ ID NO 30, SEQ ID NO 31, SEQ ID NO 32, SEQ ID NO 33 and SEQ ID NO 47.

In one embodiment the target sequence is SEQ ID NO 26, or a fragment thereof. Put another way, in some embodiments the contiguous nucleic acid is complementary to SEQ ID NO 26, or a fragment thereof.

In another embodiment the contiguous nucleotide sequence may be fully complementary to SEQ ID NO 26.

In one embodiment the target sequence is SEQ ID NO 28, or a fragment thereof. Put another way, in some embodiments the contiguous nucleic acid is complementary to SEQ ID NO 28, or a fragment thereof.

In another embodiment the contiguous nucleotide sequence may be fully complementary to SEQ ID NO 28.

In one embodiment the target sequence is SEQ ID NO 29, or a fragment thereof. Put another way, in some embodiments the contiguous nucleic acid is complementary to SEQ ID NO 29, or a fragment thereof.

In another embodiment the contiguous nucleotide sequence may be fully complementary to SEQ ID NO 29.

In one embodiment the target sequence is SEQ ID NO 30, or a fragment thereof. Put another way, in some embodiments the contiguous nucleic acid is complementary to SEQ ID NO 30, or a fragment thereof.

In another embodiment the contiguous nucleotide sequence may be fully complementary to SEQ ID NO 30.

In one embodiment the target sequence is SEQ ID NO 31, or a fragment thereof. Put another way, in some embodiments the contiguous nucleic acid is complementary to SEQ ID NO 31, or a fragment thereof.

In another embodiment the contiguous nucleotide sequence may be fully complementary to SEQ ID NO 31.

In one embodiment the target sequence is SEQ ID NO 32, or a fragment thereof. Put another way, in some embodiments the contiguous nucleic acid is complementary to SEQ ID NO 32, or a fragment thereof.

In another embodiment the contiguous nucleotide sequence may be fully complementary to SEQ ID NO 32.

In one embodiment the target sequence is SEQ ID NO 33, or a fragment thereof. Put another way, in some embodiments the contiguous nucleic acid is complementary to SEQ ID NO 33, or a fragment thereof.

In another embodiment the contiguous nucleotide sequence may be fully complementary to SEQ ID NO 33.

In one embodiment the target sequence is SEQ ID NO 38, or a fragment thereof. Put another way, in some embodiments the contiguous nucleic acid is complementary to SEQ ID NO 38, or a fragment thereof.

In another embodiment the contiguous nucleotide sequence may be fully complementary to SEQ ID NO 38.

In one embodiment the target sequence is SEQ ID NO 39, or a fragment thereof. Put another way, in some embodiments the contiguous nucleic acid is complementary to SEQ ID NO 39, or a fragment thereof.

In another embodiment the contiguous nucleotide sequence may be fully complementary to SEQ ID NO 39

In one embodiment the target sequence is SEQ ID NO 46, or a fragment thereof. Put another way, in some embodiments the contiguous nucleic acid is complementary to SEQ ID NO 46, or a fragment thereof.

In another embodiment the contiguous nucleotide sequence may be fully complementary to SEQ ID NO 46.

In one embodiment the target sequence is SEQ ID NO 47, or a fragment thereof. Put another way, in some embodiments the contiguous nucleic acid is complementary to SEQ ID NO 47, or a fragment thereof.

In another embodiment the contiguous nucleotide sequence may be fully complementary to SEQ ID NO 47.

In one embodiment the target sequence is SEQ ID NO 48, or a fragment thereof. Put another way, in some embodiments the contiguous nucleic acid is complementary to SEQ ID NO 48, or a fragment thereof.

In another embodiment the contiguous nucleotide sequence may be fully complementary to SEQ ID NO 48.

In one embodiment the target sequence is SEQ ID NO 52, or a fragment thereof. Put another way, in some embodiments the contiguous nucleic acid is complementary to SEQ ID NO 52, or a fragment thereof.

In another embodiment the contiguous nucleotide sequence may be fully complementary to SEQ ID NO 52.

In one embodiment the target sequence is SEQ ID NO 53, or a fragment thereof. Put another way, in some embodiments the contiguous nucleic acid is complementary to SEQ ID NO 53, or a fragment thereof.

In another embodiment the contiguous nucleotide sequence may be fully complementary to SEQ ID NO 53.

In one embodiment the target sequence is SEQ ID NO 55, or a fragment thereof. Put another way, in some embodiments the contiguous nucleic acid is complementary to SEQ ID NO 55, or a fragment thereof.

In another embodiment the contiguous nucleotide sequence may be fully complementary to SEQ ID NO 55.

In one embodiment the target sequence is SEQ ID NO 72, or a fragment thereof. Put another way, in some embodiments the contiguous nucleic acid is complementary to SEQ ID NO 72, or a fragment thereof.

In another embodiment the contiguous nucleotide sequence may be fully complementary to SEQ ID NO 72.

In some embodiments the fragment of any of the target sequences may be at least 8, at least 9, at least 10, at least 11, at least 12, at least 13, at least 14, at least 15, at least 16, or at least 17 contiguous nucleotides, preferably at least 10 contiguous nucleotides thereof.

### Complementarity

The term “complementarity” describes the capacity for Watson-Crick base-pairing of nucleosides/nucleotides. Watson-Crick base pairs are guanine (G)-cytosine (C) and adenine (A)-thymine (T)/uracil (U).

It will be understood that oligonucleotides may comprise nucleosides with modified nucleobases, for example 5-methyl cytosine is often used in place of cytosine, and as such the term “complementarity” encompasses Watson Crick base-paring between non-modified and modified nucleobases (see for example Hirao et al., 2012, Accounts of Chemical Research, 45, 2055 and Bergstrom, 2009, Curr. Protoc. Nucleic Acid Chem., 37, 1.4.1).

The term “% complementary” as used herein, refers to the proportion of nucleotides (in percent) of a contiguous nucleotide sequence in a nucleic acid molecule (e.g. oligonucleotide) which across the contiguous nucleotide sequence, are complementary to a reference sequence (e.g. a target sequence or sequence motif). The percentage of complementarity is thus calculated by counting the number of aligned nucleobases that are complementary (from Watson Crick base pairs) between the two sequences (when aligned with the target sequence 5′-3′ and the oligonucleotide sequence from 3′-5′), dividing that number by the total number of nucleotides in the oligonucleotide and multiplying by 100. In such a comparison a nucleobase/nucleotide which does not align (form a base pair) is termed a mismatch. Insertions and deletions are not allowed in the calculation of % complementarity of a contiguous nucleotide sequence. It will be understood that in determining complementarity, chemical modifications of the nucleobases are disregarded as long as the functional capacity of the nucleobase to form Watson Crick base pairing is retained (e.g. 5′-methyl cytosine is considered identical to a cytosine for the purpose of calculating % identity).

Within the present invention, the term “complementary” requires the antisense oligonucleotide splice modulator, or contiguous nucleotide sequence thereof, to be at least about 75% complementary, at least about 80% complementary, at least about 85% complementary, at least about 90% complementary, or at least about 95% complementary to the target sequence, e.g. the ACTL6B precursor-mRNA. In some embodiments the antisense oligonucleotide splice modulator, or contiguous sequence thereof, may be at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98% or at least about 99% complementary, or 100% complementary to the target sequence, e.g. the ACTL6B precursor-mRNA.

In some embodiments, the antisense oligonucleotide splice modulator, or contiguous nucleotide sequence thereof, of the invention may include one, two, three or more mis-matches, wherein a mis-match is a nucleotide within the antisense oligonucleotide splice modulator, or contiguous nucleotide sequence thereof, which does not base pair with its target.

The term “fully complementary”, refers to 100% complementarity.

In some embodiments the antisense oligonucleotide splice modulator is fully complementary to the target sequence.

In some embodiments the contiguous nucleotide sequence is fully complementary to the target sequence.

### Identity

The term “identity” as used herein, refers to the proportion of nucleotides (expressed in percent) of a contiguous nucleotide sequence in a nucleic acid molecule (e.g. antisense oligonucleotide splice modulator) which across the contiguous nucleotide sequence, are identical to a reference sequence (e.g. a sequence motif).

The percentage of identity is thus calculated by counting the number of aligned nucleobases that are identical (a match) between two sequences (in the contiguous nucleotide sequence of the antisense oligonucleotide splice modulator of the invention and in the reference sequence), dividing that number by the total number of nucleotides in the contiguous nucleotide sequence and multiplying by 100. Therefore, percentage of identity=(matches×100)/length of aligned region (e.g. the contiguous nucleotide sequence). Insertions and deletions are not allowed in the calculation the percentage of identity of a contiguous nucleotide sequence. It will be understood that in determining identity, chemical modifications of the nucleobases are disregarded as long as the functional capacity of the nucleobase to form Watson Crick base pairing is retained (e.g. 5-methyl cytosine is considered identical to a cytosine for the purpose of calculating % identity).

It is therefore to be understood that there is a relationship between identity and complementarity such that contiguous nucleotide sequences within the antisense oligonucleotide splice modulators of the invention that are complementary to a target sequence also share a percentage of identity with said target sequence.

### Hybridization

The terms “hybridizing” or “hybridizes” as used herein are to be understood as two nucleic acid strands (e.g. an oligonucleotide and a target nucleic acid) forming hydrogen bonds between base pairs on opposite strands thereby forming a duplex. The affinity of the binding between two nucleic acid strands is the strength of the hybridization. It is often described in terms of the melting temperature (Tm) defined as the temperature at which half of the oligonucleotides are duplexed with the target nucleic acid. At physiological conditions Tm is not strictly proportional to the affinity (Mergny and Lacroix, 2003, Oligonucleotides 13:515-537). The standard state Gibbs free energy ΔG° is a more accurate representation of binding affinity and is related to the dissociation constant (Kd) of the reaction by ΔG°=−RTIn(Kd), where R is the gas constant and T is the absolute temperature. Therefore, a very low ΔG° of the reaction between an oligonucleotide and the target nucleic acid reflects a strong hybridization between the oligonucleotide and target nucleic acid. ΔG° is the energy associated with a reaction where aqueous concentrations are 1M, the pH is 7, and the temperature is 37° C. The hybridization of oligonucleotides to a target nucleic acid is a spontaneous reaction and for spontaneous reactions ΔG° is less than zero. ΔG° can be measured experimentally, for example, by use of the isothermal titration calorimetry (ITC) method as described in Hansen et al., 1965, Chem. Comm. 36-38 and Holdgate et al., 2005, Drug Discov Today. The skilled person will know that commercial equipment is available for ΔG° measurements. ΔG° can also be estimated numerically by using the nearest neighbour model as described by SantaLucia, 1998, Proc Natl Acad Sci USA. 95: 1460-1465 using appropriately derived thermodynamic parameters described by Sugimoto et al., 1995, Biochemistry 34:11211-11216 and McTigue et al., 2004, Biochemistry 43:5388-5405.

In some embodiments, antisense oligonucleotide splice modulators of the present invention hybridize to a target nucleic acid with estimated ΔG° values below −10 kcal for oligonucleotides that are 10-30 nucleotides in length.

In some embodiments the degree or strength of hybridization is measured by the standard state Gibbs free energy ΔG°. The antisense oligonucleotide splice modulators may hybridize to a target nucleic acid with estimated ΔG° values below the range of −10 kcal, such as below −15 kcal, such as below-20 kcal and such as below −25 kcal for oligonucleotides that are 8-30 nucleotides in length. In some embodiments the antisense oligonucleotide splice modulators hybridize to a target nucleic acid with an estimated ΔG° value of −10 to −60 kcal, such as −12 to −40, such as from −15 to −30 kcal, or −16 to −27 kcal such as −18 to −25 kcal.

### Antisense Oligonucleotide Splice Modulators

The antisense oligonucleotide of the invention is an antisense oligonucleotide splice modulator comprising a contiguous nucleotide sequence which is complementary to the ACTL6B precursor-mRNA.

In some embodiments the contiguous nucleotide sequence is a sequence selected from the group consisting of SEQ ID NO 98, SEQ ID NO 99, SEQ ID NO 100, SEQ ID NO 101, SEQ ID NO 102, SEQ ID NO 103, SEQ ID NO 104, SEQ ID NO 105, SEQ ID NO 106, SEQ ID NO 107, SEQ ID NO 108, SEQ ID NO 109, SEQ ID NO 110, SEQ ID NO 111, SEQ ID NO 112, SEQ ID NO 113, SEQ ID NO 114, SEQ ID NO 115, SEQ ID NO 116, SEQ ID NO 117, SEQ ID NO 118, SEQ ID NO 119, SEQ ID NO 120, SEQ ID NO 121, SEQ ID NO 122, SEQ ID NO 123, SEQ ID NO 124, SEQ ID NO 125, SEQ ID NO 126, SEQ ID NO 127, SEQ ID NO 128, SEQ ID NO 129, SEQ ID NO 130, SEQ ID NO 131, SEQ ID NO 132, SEQ ID NO 133, SEQ ID NO 134, SEQ ID NO 135, SEQ ID NO 136, SEQ ID NO 137, SEQ ID NO 138, SEQ ID NO 139, SEQ ID NO 140, SEQ ID NO 141, SEQ ID NO 142, SEQ ID NO 143, SEQ ID NO 144, SEQ ID NO 145, SEQ ID NO 146, SEQ ID NO 147, SEQ ID NO 148, SEQ ID NO 149, SEQ ID NO 150, SEQ ID NO 151, SEQ ID NO 152, SEQ ID NO 153, SEQ ID NO 154, SEQ ID NO 155, SEQ ID NO 156, SEQ ID NO 157, SEQ ID NO 158, SEQ ID NO 159, SEQ ID NO 160, SEQ ID NO 161, SEQ ID NO 162, SEQ ID NO 163, SEQ ID NO 164, SEQ ID NO 165, SEQ ID NO 166, SEQ ID NO 167, SEQ ID NO 168, SEQ ID NO 168, SEQ ID NO 170, SEQ ID NO 171, SEQ ID NO 172, SEQ ID NO 173, SEQ ID NO 174, SEQ ID NO 175, SEQ ID NO 176, SEQ ID NO 177, SEQ ID NO 178, SEQ ID NO 179, SEQ ID NO 180, SEQ ID NO 181, SEQ ID NO 182, SEQ ID NO 183, SEQ ID NO 184, SEQ ID NO 185, SEQ ID NO 186, SEQ ID NO 187, SEQ ID NO 189, SEQ ID NO 194, SEQ ID NO 195, SEQ ID NO 196, and SEQ ID NO 197, or a fragment thereof.

In some embodiments the fragment may be at least 8, at least 9, at least 10, at least 11, at least 12, at least 13, at least 14, at least 15, at least 16, or at least 17 contiguous nucleotides of the contiguous nucleotide sequence, preferably at least 10 contiguous nucleotides thereof.

In some embodiments the contiguous nucleotide sequence is a sequence selected from the group consisting of SEQ ID NO 118, SEQ ID NO 120, SEQ ID NO 121, SEQ ID NO 122, SEQ ID NO 123, SEQ ID NO 124, SEQ ID NO 125, SEQ ID NO 130, SEQ ID NO 131, SEQ ID NO 138, SEQ ID NO 139, SEQ ID NO 140, SEQ ID NO 144, SEQ ID NO 145, SEQ ID NO 147, SEQ ID NO 164, or a fragment thereof.

In some embodiments the fragment may be at least 8, at least 9, at least 10, at least 11, at least 12, at least 13, at least 14, at least 15, at least 16, or at least 17 contiguous nucleotides of the contiguous nucleotide sequence, preferably at least 10 contiguous nucleotides thereof.

In some embodiments the contiguous nucleotide sequence is a sequence selected from the group consisting of SEQ ID NO 118, SEQ ID NO 120, SEQ ID NO 121, SEQ ID NO 122, SEQ ID NO 123, SEQ ID NO 124, SEQ ID NO 125 and SEQ ID NO 139, or a fragment thereof.

In some embodiments the fragment may be at least 8, at least 9, at least 10, at least 11, at least 12, at least 13, at least 14, at least 15, at least 16, or at least 17 contiguous nucleotides of the contiguous nucleotide sequence, preferably at least 10 contiguous nucleotides thereof.

In one embodiment the contiguous nucleotide sequence comprises SEQ ID NO 118, or a fragment thereof.

In one embodiment the contiguous nucleotide sequence consists of SEQ ID NO 118, or a fragment thereof.

In one embodiment the contiguous nucleotide sequence comprises SEQ ID NO 120, or a fragment thereof.

In one embodiment the contiguous nucleotide sequence consists of SEQ ID NO 120, or a fragment thereof.

In one embodiment the contiguous nucleotide sequence comprises SEQ ID NO 121, or a fragment thereof.

In one embodiment the contiguous nucleotide sequence consists of SEQ ID NO 121, or a fragment thereof.

In one embodiment the contiguous nucleotide sequence comprises SEQ ID NO 122, or a fragment thereof.

In one embodiment the contiguous nucleotide sequence consists of SEQ ID NO 122, or a fragment thereof.

In one embodiment the contiguous nucleotide sequence comprises SEQ ID NO 123, or a fragment thereof.

In one embodiment the contiguous nucleotide sequence consists of SEQ ID NO 123, or a fragment thereof.

In one embodiment the contiguous nucleotide sequence comprises SEQ ID NO 124, or a fragment thereof.

In one embodiment the contiguous nucleotide sequence consists of SEQ ID NO 124, or a fragment thereof.

In one embodiment the contiguous nucleotide sequence comprises SEQ ID NO 125, or a fragment thereof.

In one embodiment the contiguous nucleotide sequence consists of SEQ ID NO 125, or a fragment thereof.

In one embodiment the contiguous nucleotide sequence comprises SEQ ID NO 130, or a fragment thereof.

In one embodiment the contiguous nucleotide sequence consists of SEQ ID NO 130, or a fragment thereof.

In one embodiment the contiguous nucleotide sequence comprises SEQ ID NO 131, or a fragment thereof.

In one embodiment the contiguous nucleotide sequence consists of SEQ ID NO 131, or a fragment thereof.

In one embodiment the contiguous nucleotide sequence comprises SEQ ID NO 138, or a fragment thereof.

In one embodiment the contiguous nucleotide sequence consists of SEQ ID NO 138, or a fragment thereof.

In one embodiment the contiguous nucleotide sequence comprises SEQ ID NO 139, or a fragment thereof.

In one embodiment the contiguous nucleotide sequence consists of SEQ ID NO 139, or a fragment thereof.

In one embodiment the contiguous nucleotide sequence comprises SEQ ID NO 140, or a fragment thereof.

In one embodiment the contiguous nucleotide sequence consists of SEQ ID NO 140, or a fragment thereof.

In one embodiment the contiguous nucleotide sequence comprises SEQ ID NO 144, or a fragment thereof.

In one embodiment the contiguous nucleotide sequence consists of SEQ ID NO 144, or a fragment thereof.

In one embodiment the contiguous nucleotide sequence comprises SEQ ID NO 145, or a fragment thereof.

In one embodiment the contiguous nucleotide sequence consists of SEQ ID NO 145, or a fragment thereof.

In one embodiment the contiguous nucleotide sequence comprises SEQ ID NO 147, or a fragment thereof.

In one embodiment the contiguous nucleotide sequence consists of SEQ ID NO 147, or a fragment thereof.

In one embodiment the contiguous nucleotide sequence comprises SEQ ID NO 164, or a fragment thereof.

In one embodiment the contiguous nucleotide sequence consists of SEQ ID NO 164, or a fragment thereof.

In some embodiments the fragment may be at least 8, at least 9, at least 10, at least 11, at least 12, at least 13, at least 14, at least 15, at least 16, or at least 17 contiguous nucleotides of the contiguous nucleotide sequence, preferably at least 10 contiguous nucleotides thereof.

### Nucleotides and Nucleosides

Nucleotides and nucleosides are the building blocks of oligonucleotides and polynucleotides and, for the purposes of the present invention, include both naturally occurring and non-naturally occurring nucleotides and nucleosides. In nature, nucleotides, such as DNA and RNA nucleotides comprise a ribose sugar moiety, a nucleobase moiety and one or more phosphate groups (which is absent in nucleosides). Nucleosides and nucleotides may also interchangeably be referred to as “units” or “monomers”.

### Modified Nucleoside

The term “modified nucleoside” or “nucleoside modification” as used herein refers to nucleosides modified as compared to the equivalent DNA or RNA nucleoside by the introduction of one or more modifications of the sugar moiety or the (nucleo)base moiety.

Advantageously, the antisense oligonucleotide splice modulators according to the invention may comprise one or more modified nucleosides.

In some embodiments the antisense oligonucleotide splice modulator or the contiguous nucleotide sequence thereof (motif sequence) can be modified to, for example, increase nuclease resistance and/or binding affinity to the target nucleic acid. Advantageously, high affinity modified nucleosides are used.

Advantageously, one or more of the modified nucleosides of the antisense oligonucleotide splice modulator according to the invention may comprise a modified sugar moiety. The term modified nucleoside may also be used herein interchangeably with the term “nucleoside analogue” or modified “units” or modified “monomers”. Nucleosides with an unmodified DNA or RNA sugar moiety are termed DNA or RNA nucleosides herein. Nucleosides with modifications in the base region of the DNA or RNA nucleoside are still generally termed DNA or RNA if they allow Watson Crick base pairing. Exemplary modified nucleosides which may be used in the antisense oligonucleotide splice modulators according to the invention include LNA, 2′-O-MOE, 2′oMe and morpholino nucleoside analogues.

### Modified Internucleoside Linkage

Advantageously, the antisense oligonucleotide splice modulators according to the invention comprise one or more modified internucleoside linkages.

The term “modified internucleoside linkage” is defined as generally understood by the skilled person as linkages, other than phosphodiester (PO) linkages, which covalently couple two nucleosides together. The antisense oligonucleotide splice modulator of the invention may therefore comprise one or more modified internucleoside linkages such as one or more phosphorothioate internucleoside linkages.

In some embodiments at least 50% of the internucleoside linkages in the antisense oligonucleotide splice modulators according to the invention, or the contiguous nucleotide sequence thereof, are phosphorothioate, such as at least 60%, such as at least 70%, such as at least 75%, such as at least 80%, such as at least 90% or more. In some embodiments all of the internucleoside linkages of the antisense oligonucleotide splice modulators of the invention, or contiguous nucleotide sequence thereof, are phosphorothioate.

In a further embodiment, the antisense oligonucleotide splice modulators according to the invention, or the contiguous nucleotide sequence thereof, comprise at least one modified internucleoside linkage. It is advantageous if at least 75%, such as all, of the internucleoside linkages within the contiguous nucleotide sequence are phosphorothioate or boranophosphate internucleoside linkages.

Advantageously, all the internucleoside linkages of the contiguous nucleotide sequence of the antisense oligonucleotide splice modulators according to the invention may be phosphorothioate, or all the internucleoside linkages of the antisense oligonucleotide splice modulators according to the invention may be phosphorothioate linkages.

### Nucleobase

The term nucleobase includes the purine (e.g. adenine and guanine) and pyrimidine (e.g. uracil, thymine and cytosine) moiety present in nucleosides and nucleotides which form hydrogen bonds in nucleic acid hybridization. In the context of the present invention, the term nucleobase also encompasses modified nucleobases which may differ from naturally occurring nucleobases, but which are functional during nucleic acid hybridisation. In this context “nucleobase” refers to both naturally occurring nucleobases such as adenine, guanine, cytosine, thymidine, uracil, xanthine and hypoxanthine, as well as non-naturally occurring variants. Such variants are for example described in Hirao et al. (2012) Accounts of Chemical Research vol 45 page 2055 and Bergstrom (2009) Current Protocols in Nucleic Acid Chemistry Suppl. 37 1.4.1.

In some embodiments the nucleobase moiety is modified by changing the purine or pyrimidine into a modified purine or pyrimidine, such as substituted purine or substituted pyrimidine, such as a nucleobase selected from isocytosine, pseudoisocytosine, 5-methyl cytosine, 5-thiozolo-cytosine, 5-propynyl-cytosine, 5-propynyl-uracil, 5-bromouracil 5-thiazolo-uracil, 2-thio-uracil, 2′thio-thymine, inosine, diaminopurine, 6-aminopurine, 2-aminopurine, 2,6-diaminopurine and 2-chloro-6-aminopurine.

The nucleobase moieties may be indicated by the letter code for each corresponding nucleobase, e.g. A, T, G, C or U, wherein each letter may optionally include modified nucleobases of equivalent function. For example, in the exemplified oligonucleotides, the nucleobase moieties are selected from A, T, G, C, and 5-methyl cytosine.

### Modified Oligonucleotide

The antisense oligonucleotide splice modulators of the invention may be modified oligonucleotides.

The term “modified oligonucleotide” describes an oligonucleotide comprising one or more sugar-modified nucleosides and/or modified internucleoside linkages. The term “chimeric oligonucleotide” is a term that has been used in the literature to describe oligonucleotides comprising sugar modified nucleosides and DNA nucleosides. In some embodiments, it may be advantageous for the antisense oligonucleotide splice modulators according to the invention to be or to comprise chimeric oligonucleotides.

In some embodiments, antisense oligonucleotide splice modulators according to the invention, or contiguous nucleotide sequence thereof, may include modified nucleobases, which function as the shown nucleobase in base pairing, for example 5-methyl cytosine may be used in place of methyl cytosine. Inosine may be used as a universal base.

It is understood that the contiguous nucleobase sequences (motif sequence) can be modified to, for example, increase nuclease resistance and/or binding affinity to the target nucleic acid.

The pattern in which the modified nucleosides (such as high affinity modified nucleosides) are incorporated into an oligonucleotide sequence is generally termed oligonucleotide design.

In an embodiment, the antisense oligonucleotide splice modulators according to the invention comprise at least 1 modified nucleoside, such as at least 2, at least 3, at least 4, at least 5, at least 6, at least 7, at least 8, at least 9, at least 10, at least 11, at least 12, at least 13, at least 14, at least 15, at least 16, at least 17, at least 18, or at least 19 modified nucleosides.

Suitable modifications are described herein under the headings “modified nucleoside”, “high affinity modified nucleosides”, “sugar modifications”, “2′ sugar modifications” and “Locked nucleic acids (LNA)”.

### High Affinity Modified Nucleosides

A high affinity modified nucleoside is a modified nucleoside which, when incorporated into the oligonucleotide enhances the affinity of the oligonucleotide for its complementary target, for example as measured by the melting temperature (Tm). A high affinity modified nucleoside of the present invention preferably results in an increase in melting temperature between +0.5 to +12° C., more preferably between +1.5 to +10° C. and most preferably between +3 to +8° C. per modified nucleoside. Numerous high affinity modified nucleosides are known in the art and include for example, many 2′ substituted nucleosides as well as locked nucleic acids (LNA) (see e.g. Freier & Altmann; Nucl. Acid Res., 1997, 25, 4429-4443 and Uhlmann; Curr. Opinion in Drug Development, 2000, 3(2), 203-213).

### Sugar Modifications

The antisense oligonucleotide splice modulators according to the invention may comprise one or more nucleosides which have a modified sugar moiety, i.e. a modification of the sugar moiety when compared to the ribose sugar moiety found in DNA and RNA.

Numerous nucleosides with modification of the ribose sugar moiety have been made, primarily with the aim of improving certain properties of oligonucleotides, such as affinity and/or nuclease resistance.

Such modifications include those where the ribose ring structure is modified, e.g. by replacement with a hexose ring (HNA), or a bicyclic ring, which typically have a biradicle bridge between the C2 and C4 carbons on the ribose ring (LNA), or an unlinked ribose ring which typically lacks a bond between the C2 and C3 carbons (e.g. UNA). Other sugar modified nucleosides include, for example, bicyclohexose nucleic acids (WO2011/017521) or tricyclic nucleic acids (WO2013/154798). Modified nucleosides also include nucleosides where the sugar moiety is replaced with a non-sugar moiety, for example in the case of peptide nucleic acids (PNA), or morpholino nucleic acids.

Sugar modifications also include modifications made via altering the substituent groups on the ribose ring to groups other than hydrogen, or the 2′—OH group naturally found in DNA and RNA nucleosides. Substituents may, for example be introduced at the 2′, 3′, 4′ or 5′ positions.

### 2′ Sugar Modified Nucleosides

A 2′ sugar modified nucleoside is a nucleoside which has a substituent other than H or —OH at the 2′ position (2′ substituted nucleoside) or comprises a 2′ linked biradicle capable of forming a bridge between the 2′ carbon and a second carbon in the ribose ring, such as LNA (2′-4′ biradicle bridged) nucleosides.

Indeed, much focus has been spent on developing 2′ sugar substituted nucleosides, and numerous 2′ substituted nucleosides have been found to have beneficial properties when incorporated into oligonucleotides. For example, the 2′ modified sugar may provide enhanced binding affinity and/or increased nuclease resistance to the oligonucleotide. Examples of 2′ substituted modified nucleosides are 2′-O-alkyl-RNA, 2′-O-methyl-RNA (2′oMe), 2′-alkoxy-RNA, 2′-O-methoxyethyl-RNA (MOE), 2′-amino-DNA, 2′-Fluoro-RNA, and 2′-F-ANA nucleoside. For further examples, please see e.g. Freier & Altmann; Nucl. Acid Res., 1997, 25, 4429-4443 and Uhlmann; Curr. Opinion in Drug Development, 2000, 3(2), 203-213, and Deleavey and Damha, Chemistry and Biology 2012, 19, 937. Below are illustrations of some 2′ substituted modified nucleosides.

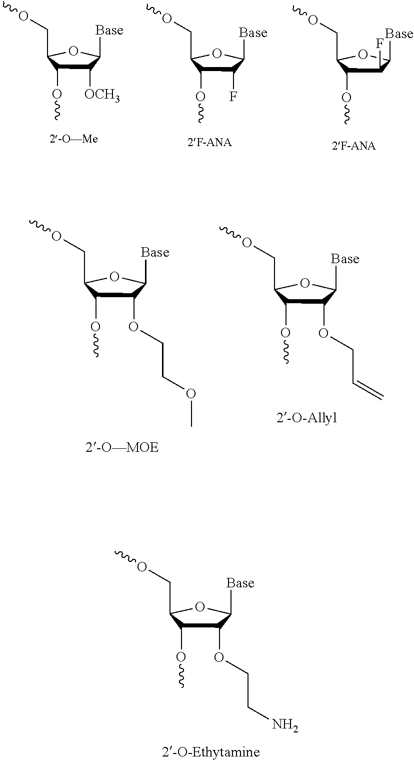

In relation to the present invention, 2′ substituted sugar modified nucleosides does not include 2′ bridged nucleosides like LNA.

In one embodiment, the antisense oligonucleotide splice modulators according to the invention may comprise one or more sugar modified nucleosides, such as 2′ sugar modified nucleosides. Preferably the antisense oligonucleotide splice modulators according to the invention comprises one or more 2′ sugar modified nucleoside independently selected from the group consisting of 2′-O-alkyl-RNA, 2′-O-methyl-RNA (2′oMe), 2′-alkoxy-RNA, 2′-O-methoxyethyl-RNA (2′MOE), 2′-amino-DNA, 2′-fluoro-DNA, arabino nucleic acid (ANA), 2′-fluoro-ANA and LNA nucleosides. It is advantageous if one or more of the modified nucleoside(s) is a locked nucleic acid (LNA).

### Locked Nucleic Acid Nucleosides (LNA Nucleoside)

A “LNA nucleoside” is a 2′-modified nucleoside which comprises a biradical linking the C2′ and C4′ of the ribose sugar ring of said nucleoside (also referred to as a “2′-4′ bridge”), which restricts or locks the conformation of the ribose ring. These nucleosides are also termed bridged nucleic acid or bicyclic nucleic acid (BNA) in the literature. The locking of the conformation of the ribose is associated with an enhanced affinity of hybridization (duplex stabilization) when the LNA is incorporated into an oligonucleotide for a complementary RNA or DNA molecule. This can be routinely determined by measuring the melting temperature of the oligonucleotide/complement duplex.

Non limiting, exemplary LNA nucleosides are disclosed in WO 99/014226, WO 00/66604, WO 98/039352, WO 2004/046160, WO 00/047599, WO 2007/134181, WO 2010/077578, WO 2010/036698, WO 2007/090071, WO 2009/006478, WO 2011/156202, WO 2008/154401, WO 2009/067647, WO 2008/150729, Morita et al., Bioorganic & Med. Chem. Lett. 12, 73-76, Seth et al. J. Org. Chem. 2010, Vol 75(5) pp. 1569-81, and Mitsuoka et al., Nucleic Acids Research 2009, 37(4), 1225-1238, and Wan and Seth, J. Medical Chemistry 2016, 59, 9645-9667.

Further non limiting, exemplary LNA nucleosides are disclosed in Scheme 1.

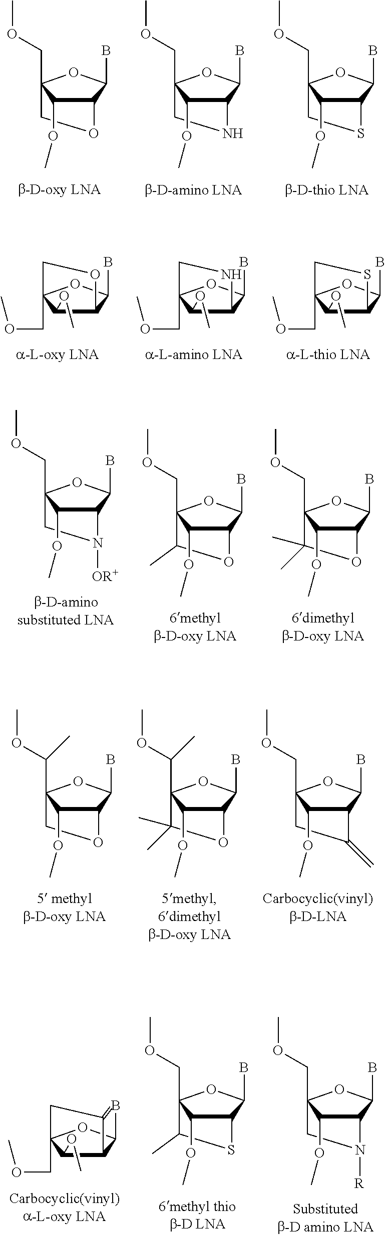

Particular LNA nucleosides are beta-D-oxy-LNA, 6′-methyl-beta-D-oxy LNA such as (S)-6′-methyl-beta-D-oxy-LNA (ScET) and ENA.

A particularly advantageous LNA is beta-D-oxy-LNA.

### Morpholino Oligonucleotides

In some embodiments, the antisense oligonucleotide splice modulators of the invention comprise or consist of morpholino nucleosides (i.e. are Morpholino oligomers and phosphorodiamidate Morpholino oligomer (PMO)). Splice modulating morpholino oligonucleotides have been approved for clinical use-see for example eteplirsen, a 30 nucleotide morpholino oligonucleotide targeting a frame shift mutation in DMD, used to treat Duchenne muscular dystrophy. Morpholino oligonucleotides have nucleases attached to six membered morpholino rings rather ribose, such as methylenemorpholine rings linked through phosphorodiamidate groups, for example as illustrated by the following illustration of 4 consecutive morpholino nucleotides:

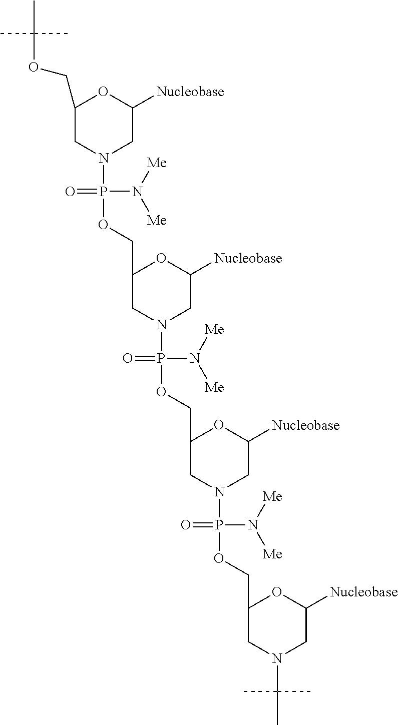

In some embodiments, antisense oligonucleotide splice modulators according to the invention may be, for example 8 to 40 morpholino nucleotides in length, such as morpholino 16 to 20 nucleotides in length, such as 18 to 20 nucleotides in length.

### RNase H Activity and Recruitment

The RNase H activity of an antisense oligonucleotide refers to its ability to recruit RNase H when in a duplex with a complementary RNA molecule. WO01/23613 provides in vitro methods for determining RNase H activity, which may be used to determine the ability to recruit RNase H. Typically an oligonucleotide is deemed capable of recruiting RNase H if it, when provided with a complementary target nucleic acid sequence, has an initial rate, as measured in pmol/l/min, of at least 5%, such as at least 10%, at least 20% or more than 20%, of the initial rate determined when using an oligonucleotide having the same base sequence as the modified oligonucleotide being tested, but containing only DNA monomers with phosphorothioate linkages between all monomers in the oligonucleotide, and using the methodology provided by Examples 91-95 of WO01/23613 (hereby incorporated by reference). For use in determining RHase H activity, recombinant RNase H1 is available from Lubio Science GmbH, Lucerne, Switzerland.

DNA oligonucleotides are known to effectively recruit RNase H, as are gapmer oligonucleotides which comprise a region of DNA nucleosides (typically at least 5 or 6 contiguous DNA nucleosides), flanked 5′ and 3′ by regions comprising 2′ sugar modified nucleosides, typically high affinity 2′ sugar modified nucleosides, such as 2-O-MOE and/or LNA. For effective function as a splice modulator, degradation of the precursor-mRNA is not desirable, and as such it is preferable to avoid the RNaseH degradation of the target. Therefore, the antisense oligonucleotide splice modulators of the invention are preferably not RNase H recruiting gapmer oligonucleotides.

RNase H recruitment may be avoided by limiting the number of contiguous DNA nucleotides in the antisense oligonucleotide splice modulator-therefore mixmer and totalmer designs may be used. Advantageously, in some embodiments, the antisense oligonucleotide splice modulators of the invention, or the contiguous nucleotide sequences thereof, does not comprise more than 3 contiguous DNA nucleosides. Further, advantageously, in some embodiments, the antisense oligonucleotide splice modulators of the invention, or the contiguous nucleotide sequences thereof, do not comprise more than 4 contiguous DNA nucleosides. Further advantageously, in some embodiments, the antisense oligonucleotide splice modulators of the invention, or contiguous nucleotide sequences thereof, do not comprise more than 2 contiguous DNA nucleosides.

### Mixmers and Totalmers

For use as a splice modulator it is often advantageous to use antisense oligonucleotides which do not recruit RNAase H and do not cause destruction of target pre-cursor-RNA. As RNase H activity requires a contiguous sequence of DNA nucleotides, RNase H recruitment may be prevented by designing oligonucleotides which do not comprise a region of more than 3 or more than 4 contiguous DNA nucleosides. This may be achieved by using antisense oligonucleotides or contiguous nucleoside regions thereof with a mixmer design, which comprise sugar modified nucleosides, such as 2′ sugar modified nucleosides, and short regions of DNA nucleosides, such as 1, 2 or 3 DNA nucleosides. Mixmers are exemplified herein by every second design, wherein the nucleosides alternate between 1 LNA and 1 DNA nucleoside, e.g. LDLDLDLDLDLDLDLL, with 5′ and 3′ terminal LNA nucleosides, and every third design, such as LDDLDDLDDLDDLDDL, where every third nucleoside is a LNA nucleoside.

In one embodiment, the mixmer may comprise or consist of nucleosides that alternate between 1, 2 or 3 sequential DNA nucleosides, followed by 1 or 2 sequential LNA nucleosides.

A totalmer is an oligonucleotide or a contiguous nucleotide sequence thereof which does not comprise DNA or RNA nucleosides, and may for example comprise only 2′-O-MOE nucleosides, such as a fully MOE phosphorothioate, e.g. MMMMMMMMMMMMMMMMMMMM, where M=2′-O-MOE, or may for example comprise only 2′oMe nucleosides, which are reported to be effective for therapeutic use.

Alternatively, a mixmer may comprise a mixture of modified nucleosides, such as MLMLMLMLMLMLMLMLMLML, wherein L=LNA and M=2′-O-MOE nucleosides. Advantageously, the internucleoside nucleosides in mixmers and totalmers may be phosphorothioate, or a majority of nucleoside linkages in mixmers may be phosphorothioate.

Mixmers and totalmers may comprise other internucleoside linkages, such as phosphodiester or phosphorodithioate, by way of example.

In some embodiments, the antisense oligonucleotide splice modulator is or comprises an oligonucleotide mixmer or totalmer. In some embodiments, the contiguous nucleotide sequence is a mixmer or a totalmer.

### Region D′ or D″ in an Oligonucleotide

The antisense oligonucleotide splice modulators of the invention may in some embodiments comprise the contiguous nucleotide sequences of the oligonucleotides which are complementary to the target nucleic acid, such as a mixmer or totalmer region, and further 5′ and/or 3′ nucleosides. The further 5′ and/or 3′ nucleosides may or may not be complementary, such as fully complementary, to the target nucleic acid. Such further 5′ and/or 3′ nucleosides may be referred to as region D′ and D″ herein.

The addition of region D′ or D″ may be used for the purpose of joining the contiguous nucleotide sequence, such as the mixmer or totalmer, to a conjugate moiety or another functional group. When used for joining the contiguous nucleotide sequence with a conjugate moiety is can serve as a biocleavable linker. Alternatively, it may be used to provide exonucleoase protection or for ease of synthesis or manufacture.

Region D′ or D″ may independently comprise or consist of 1, 2, 3, 4 or 5 additional nucleotides, which may be complementary or non-complementary to the target nucleic acid. The nucleotide adjacent to the F or F′ region is not a sugar-modified nucleotide, such as a DNA or RNA or base modified versions of these. The D′ or D′ region may serve as a nuclease susceptible biocleavable linker (see definition of linkers). In some embodiments the additional 5′ and/or 3′ end nucleotides are linked with phosphodiester linkages, and are DNA or RNA. Nucleotide based biocleavable linkers suitable for use as region D′ or D″ are disclosed in WO2014/076195, which include by way of example a phosphodiester linked DNA dinucleotide. The use of biocleavable linkers in poly-oligonucleotide constructs is disclosed in WO2015/113922, where they are used to link multiple antisense constructs within a single oligonucleotide.

In one embodiment the antisense oligonucleotide splice modulators of the invention may comprise a region D′ and/or D″ in addition to the contiguous nucleotide sequence, which may constitute a mixmer or a totalmer.

In some embodiments the internucleoside linkage positioned between region D′ or D″ and the mixmer or totalmer region may be a phosphodiester linkage.

### Conjugates

The invention encompasses an antisense oligonucleotide splice modulator covalently attached to at least one conjugate moiety. In some embodiments this may be referred to as a conjugate of the invention.

In some embodiments, the invention provides an antisense oligonucleotide splice modulator covalently attached to at least one conjugate moiety.

The term “conjugate” as used herein refers to an antisense oligonucleotide splice modulator which is covalently linked to a non-nucleotide moiety (conjugate moiety or region C or third region). The conjugate moiety may be covalently linked to the antisense oligonucleotide splice modulator, optionally via a linker group, such as region D′ or D″.

Oligonucleotide conjugates and their synthesis has also been reported in comprehensive reviews by Manoharan in Antisense Drug Technology, Principles, Strategies, and Applications, S. T. Crooke, ed., Ch. 16, Marcel Dekker, Inc., 2001 and Manoharan, Antisense and Nucleic Acid Drug Development, 2002, 12, 103.

In some embodiments, the non-nucleotide moiety (conjugate moiety) is selected from the group consisting of carbohydrates (e.g. GalNAc), cell surface receptor ligands, drug substances, hormones, lipophilic substances, polymers, proteins, peptides, toxins (e.g. bacterial toxins), vitamins, viral proteins (e.g. capsids) or combinations thereof.

### Linkers

A linkage or linker is a connection between two atoms that links one chemical group or segment of interest to another chemical group or segment of interest via one or more covalent bonds. Conjugate moieties can be attached to the antisense oligonucleotide splice modulator directly or through a linking moiety (e.g. linker or tether). Linkers serve to covalently connect a third region, e.g. a conjugate moiety (Region C), to a first region, e.g. an antisense oligonucleotide splice modulator or contiguous nucleotide sequence complementary to the target nucleic acid (region A).

In some embodiments of the invention, the conjugate or antisense oligonucleotide splice modulator of the invention may optionally comprise a linker region (second region or region B and/or region Y) which is positioned between the antisense oligonucleotide splice modulator or contiguous nucleotide sequence complementary to the target nucleic acid (region A or first region) and the conjugate moiety (region C or third region).

Region B refers to biocleavable linkers comprising or consisting of a physiologically labile bond that is cleavable under conditions normally encountered or analogous to those encountered within a mammalian body. Conditions under which physiologically labile linkers undergo chemical transformation (e.g., cleavage) include chemical conditions such as pH, temperature, oxidative or reductive conditions or agents, and salt concentration found in or analogous to those encountered in mammalian cells. Mammalian intracellular conditions also include the presence of enzymatic activity normally present in a mammalian cell such as from proteolytic enzymes or hydrolytic enzymes or nucleases. In one embodiment the biocleavable linker is susceptible to S1 nuclease cleavage. In some embodiments the nuclease susceptible linker comprises between 1 and 5 nucleosides, such as DNA nucleoside(s) comprising at least two consecutive phosphodiester linkages. Phosphodiester containing biocleavable linkers are described in more detail in WO 2014/076195.

Region Y refers to linkers that are not necessarily biocleavable but primarily serve to covalently connect a conjugate moiety (region C or third region), to an oligonucleotide (region A or first region). The region Y linkers may comprise a chain structure or an oligomer of repeating units such as ethylene glycol, amino acid units or amino alkyl groups. The antisense oligonucleotide splice modulator conjugates of the present invention can be constructed of the following regional elements A-C, A-B-C, A-B-Y-C, A-Y-B-C or A-Y-C. In some embodiments the linker (region Y) is an amino alkyl, such as a C2-C36 amino alkyl group, including, for example C6 to C12 amino alkyl groups. In some embodiments the linker (region Y) is a C6 amino alkyl group.

### Salts

The term “salts” as used herein conforms to its generally known meaning, i.e. an ionic assembly of anions and cations.

In some embodiments, the antisense oligonucleotide splice modulator of the invention may be in the form of a pharmaceutically acceptable salt. Put another way, the invention provides for pharmaceutically acceptable salts of the antisense oligonucleotide splice modulator of the invention.

In some embodiments the pharmaceutically acceptable salt may be a sodium salt or a potassium salt.

The invention provides for a pharmaceutically acceptable sodium salt of the antisense oligonucleotide splice modulator of the invention.

The invention provides for a pharmaceutically acceptable potassium salt of the antisense oligonucleotide splice modulator of the invention.

### Delivery of Antisense Oligonucleotide Splice Modulators

The invention provides for antisense oligonucleotide splice modulators of the invention wherein the antisense oligonucleotide splice modulators are encapsulated in a lipid-based delivery vehicle, covalently linked to or encapsulated in a dendrimer, or conjugated to an aptamer.

This may be for the purpose of delivering the antisense oligonucleotide splice modulators of the invention to the targeted cells and/or to improve the pharmacokinetics of the antisense oligonucleotide splice modulator.

Examples of lipid-based delivery vehicles include oil-in-water emulsions, micelles, liposomes, and lipid nanoparticles.

### Pharmaceutical Compositions

The invention provides for a pharmaceutical composition comprising the antisense oligonucleotide splice modulator of the invention, and a pharmaceutically acceptable diluent, solvent, carrier, salt and/or adjuvant.

The invention provides for a pharmaceutical composition comprising the antisense oligonucleotide splice modulator of the invention, and a pharmaceutically acceptable salt.

For example, the salt may comprise a metal cation, such as a sodium salt or a potassium salt.

The invention provides for a pharmaceutical composition of the invention, wherein the pharmaceutical composition comprises an antisense oligonucleotide splice modulator of the invention, and an aqueous diluent or solvent.

The invention provides for a solution, such as a phosphate buffered saline solution of the antisense oligonucleotide splice modulator of the invention. In some embodiments, the solution, such as phosphate buffered saline solution, of the invention is a sterile solution.

### Method for Increasing ACTL6B Expression

The invention provides for a method for enhancing, upregulating or restoring the expression of wild-type ACTL6B in a cell, such as a cell which is expressing ACTL6B, said method comprising administering an antisense oligonucleotide splice modulator of the invention, or A pharmaceutical composition of the invention in an effective amount to said cell.

In some embodiments the method is an in vitro method.

In some embodiments the method is an in vivo method.

In some embodiments, the cell is an animal cell, preferably a mammalian cell such as a mouse cell, rat cell, hamster cell, or monkey cell, or preferably a human cell.

In some embodiments, the cell is a mammalian cell.

In some embodiments, the cell is a human cell.

In some embodiments the cell is part of, or derived from, a subject suffering from or susceptible to a disease associated with reduced expression of wild-type ACTL6B. Such diseases include but are not limited amyotrophic lateral sclerosis (ALS), frontotemporal lobar degeneration (FTLD), Progressive supranuclear palsy (PSP), Primary lateral sclerosis, Progressive muscular atrophy, Alzheimer's disease, Parkinsons disease, Autism, Hippocampal sclerosis dementia, Down syndrome, Huntington's disease, polyglutamine diseases, such as spinocerebellar ataxia 3, myopathies and Chronic Traumatic Encephalopathy.

### Treatment

The term “treatment” as used herein refers to both treatment of an existing disease (e.g. a disease or disorder as herein referred to), or prevention of a disease, i.e. prophylaxis. It will therefore be recognized that treatment, as referred to herein may in some embodiments be prophylactic.

The invention provides for a method for treating or preventing a disease, comprising administering a therapeutically or prophylactically effective amount of an antisense oligonucleotide splice modulator of the invention or a pharmaceutical composition of the invention to a subject suffering from or susceptible to a disease.

The disease may be associated with reduced expression of wild-type ACTL6B. In some embodiments, the invention provides for a method for treating or preventing a disease associated with reduced expression of wild-type ACTL6B, comprising administering a therapeutically or prophylactically effective amount of an antisense oligonucleotide splice modulator of the invention or a pharmaceutical composition of the invention to a subject suffering from or susceptible to a disease associated with reduced expression of wild-type ACTL6B.

In one embodiment, the disease is a neurological disorder.

In one embodiment the disease is selected from the group consisting of amyotrophic lateral sclerosis (ALS), frontotemporal lobar degeneration (FTLD), Progressive supranuclear palsy (PSP), Primary lateral sclerosis, Progressive muscular atrophy, Alzheimer's disease, Parkinsons disease, Autism, Hippocampal sclerosis dementia, Down syndrome, Huntington's disease, polyglutamine diseases, such as spinocerebellar ataxia 3, myopathies and Chronic Traumatic Encephalopathy.

In some embodiments, the subject is an animal, preferably a mammal such as a mouse, rat, hamster, or monkey, or human.

In some embodiments, the subject is a human.

The invention provides for an antisense oligonucleotide splice modulator of the invention for use as a medicament.

The invention provides for an antisense oligonucleotide splice modulator of the invention for the preparation of a medicament.

The invention provides for an antisense oligonucleotide splice modulator of the invention for use in therapy.

The invention provides for a pharmaceutical composition of the invention for use as a medicament.

The invention provides for a pharmaceutical composition of the invention for the preparation of a medicament.

The invention provides for a pharmaceutical composition of the invention for use in therapy.

The invention provides for an antisense oligonucleotide splice modulator of the invention for use as a medicament in the treatment of a neurological disorder.

The invention provides for an antisense oligonucleotide splice modulator of the invention for use as a medicament in the treatment of a disease selected from the group consisting of amyotrophic lateral sclerosis (ALS), frontotemporal lobar degeneration (FTLD), Progressive supranuclear palsy (PSP), Primary lateral sclerosis, Progressive muscular atrophy, Alzheimer's disease, Parkinsons disease, Autism, Hippocampal sclerosis dementia, Down syndrome, Huntington's disease, polyglutamine diseases, such as spinocerebellar ataxia 3, myopathies and Chronic Traumatic Encephalopathy.

The invention provides for the use of an antisense oligonucleotide splice modulator of the invention for the preparation of a medicament for the treatment or prevention of a neurological disorder.

The invention provides for the use of an antisense oligonucleotide splice modulator of the invention for the preparation of a medicament for the treatment or prevention of a disease selected from the group consisting of amyotrophic lateral sclerosis (ALS), frontotemporal lobar degeneration (FTLD), Progressive supranuclear palsy (PSP), Primary lateral sclerosis, Progressive muscular atrophy, Alzheimer's disease, Parkinsons disease, Autism, Hippocampal sclerosis dementia, Down syndrome, Huntington's disease, polyglutamine diseases, such as spinocerebellar ataxia 3, myopathies and Chronic Traumatic Encephalopathy.

The invention provides for a pharmaceutical composition of the invention for use as a medicament in the treatment of a neurological disorder.

The invention provides for a pharmaceutical composition of the invention for use as a medicament in the treatment of a disease selected from the group consisting of amyotrophic lateral sclerosis (ALS), frontotemporal lobar degeneration (FTLD), Progressive supranuclear palsy (PSP), Primary lateral sclerosis, Progressive muscular atrophy, Alzheimer's disease, Parkinsons disease, Autism, Hippocampal sclerosis dementia, Down syndrome, Huntington's disease, polyglutamine diseases, such as spinocerebellar ataxia 3, myopathies and Chronic Traumatic Encephalopathy.

The invention provides for the use of a pharmaceutical composition of the invention for the preparation of a medicament for the treatment or prevention of a neurological disorder.

The invention provides for the use of a pharmaceutical composition of the invention for the preparation of a medicament for the treatment or prevention of a disease selected from the group consisting of amyotrophic lateral sclerosis (ALS), frontotemporal lobar degeneration (FTLD), Progressive supranuclear palsy (PSP), Primary lateral sclerosis, Progressive muscular atrophy, Alzheimer's disease, Parkinsons disease, Autism, Hippocampal sclerosis dementia, Down syndrome, Huntington's disease, polyglutamine diseases, such as spinocerebellar ataxia 3, myopathies and Chronic Traumatic Encephalopathy.

### Administration

The antisense oligonucleotide splice modulator of the invention or the pharmaceutical composition of the invention may be administered topically (such as, to the skin, inhalation, ophthalmic or otic) or enteral (such as, orally or through the gastrointestinal tract) or parenterally (such as, intravenous, subcutaneous, intramuscular, intracerebral, intracerebroventricular or intrathecal).

In a preferred embodiment the antisense oligonucleotide splice modulator of the invention or pharmaceutical composition of the invention is administered by a parenteral route including intravenous, intraarterial, subcutaneous, intraperitoneal or intramuscular injection or infusion, intrathecal or intracranial, e.g., intracerebral or intraventricular, administration. In one embodiment the antisense oligonucleotide splice modulator of the invention is administered intracerebrally or intracerebroventricularly. In another embodiment the antisense oligonucleotide splice modulator of the invention is administered intrathecally.

The invention also provides for the use of the antisense oligonucleotide splice modulator of the invention or pharmaceutical composition of the invention as described for the preparation of a medicament wherein the medicament is in a dosage form for intrathecal administration.

The invention also provides for the use of the antisense oligonucleotide splice modulator of the invention or pharmaceutical composition of the invention as described for the preparation of a medicament wherein the medicament is in a dosage form for intracerebral or intraventricular administration.

The invention also provides for the use of the antisense oligonucleotide splice modulator of the invention or pharmaceutical composition of the invention as described for the preparation of a medicament wherein the medicament is in a dosage form for intracerebroventricular administration.

### Combination Therapies

In some embodiments, the antisense oligonucleotide splice modulator of the invention or pharmaceutical composition of the invention is for use in a combination treatment with one or more other therapeutic agents.

### EXAMPLES

### Example 1: Identification of ACTL6B as a Novel Target for TDP43 mRNA Splice Regulation

One hallmark feature of ALS disease is the presence of cytoplasmic aggregated TDP43 protein in a small fraction of the patient's neuronal cells. The consequence of cytoplasmic aggregation of TDP43 is that it becomes depleted in the cell nucleus, and hence can't perform its normal function here.

TDP43 has been shown to affect mRNA splicing. In order to identify new genes whose mRNAs are regulated by the presence of TDP43, we did a knockdown of TDP43 in a neuronal cell model. RNA sequencing was performed on the cells, and de novo transcript analysis was performed to identify affected genes with new splice patterns.

Human glutamatergic neurons (Fujifilms) were plated at 60,000 viable cells together with 10,000 viable Astrocytes (Fujifilms) in 96-well plates coated with Laminin and Poly(ethyleneimine) solution (Sigma Aldrich) in 200 μl culture medium (day-1).

To knockdown TDP-43, compound A (SEQ ID 198) was added to the culture medium at 5 μM on day 0, in other wells PBS was added instead as control. Half the cell culture medium was changed 3 times a week during the whole experiment (day 2, 5, 7, 10, 12, 14 & 17). The cells were harvested on day 20 using Magnapure lysis buffer (Roche) and RNA was isolation on MagNA pure 96 system (Roche) according to the manufacturer's instructions including DNase treatment step. NGS libraries was prepared from 100 ng of total RNA using the KAPA mRNA HyperPrep Kit Illumina® Platforms (Roche). Libraries were subjected to paired-end sequencing on a NovaSeq6000 sequencer (Illumina) with 150-bp read length. Data analysis was carried out using CLC Genomics Workbench 21. Data was first analyzed by running large gap mapping analysis using hg38 genome assembly, followed by transcript discovery. Predicted novel splice events were examined by manual visual inspection to identify real splice events.

The inclusion of a novel 69 base pair exon in ACTL6B upon loss of TDP43 was discovered. The first and last base in the new exon is 100,650,643 and 100,650,575 according to the hg38 human gene annotation with the ACTL6B being placed in the minus orientation.

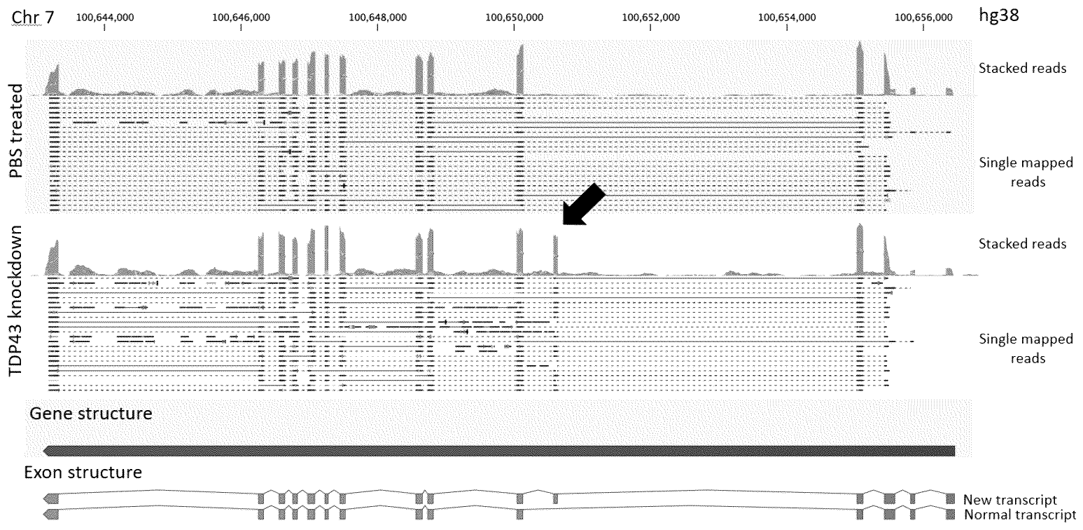

FIG. 1 displays a screenshot from the CLC Genomics Workbench software where the NGS read mapping from the ACTL6B gene can be seen. The inclusion of the new exon in the sample treated with compound A (SEQ ID 198) is shown with an arrow. Approximately 99% of all the mRNAs in this sample contains this exon. For the untreated sample the inclusion of this exon was seen in less than 0.5% of the reads.

### Example 2: Rescue of Erroneous ACTL6B mRNA Splicing Caused by the Lack of TDP43 Using ASO

Here we show ASOs ability to induce proper splicing on the TDP43 target ACTL6B. Human glutamatergic neurons (Fujifilms) were plated at 60,000 viable cells together with 10,000 viable Astrocytes (Fujifilms) in 96-well plates coated with Laminin and Poly(ethyleneimine) solution (Sigma Aldrich) in 200 μl culture medium (day-1). To knockdown TDP-43, compound A (SEQ ID 198) was added to the culture medium at 5 μM on day 0 (Except for four control wells per plate). Half the cell culture medium was changed 3 times a week during the whole experiment (day 2, 5, 7, 9, 12, 14, 16 & 19). The ASOs targeting the cryptic ACTL6B exon, was added to the culture medium on day 5 at 10 μM. 96 different ASOs were added in total (SEQ IDs 98-189 and 194-197). At least 12 wells per plate received only the compound A (SEQ ID 198) to serve as a baseline reference. The experiments were run in duplicate. With a total of four 96 well plates.

The cells were harvested on day 20 using Magnapure lysis buffer (Roche) and RNA was isolated on MagNA pure 96 system (Roche) according to the manufacturer's instructions including DNase treatment step. The purified RNA was denatured 30 sec at 90 before cDNA synthesis. cDNA was created using the iScript Advanced cDNA Synthesis Kit for RT-qPCR (Biorad) according to the manufacturer's instructions.

Measurements of the expression levels of the target genes was done by droplet digital PCR using the QX1 system (Bio-Rad) together with the QX1 software stand edition. The PCR-probe assays used to measure the expressed of normally spliced target mRNA was designed to span the two exons, where in-between the new “mutant” exon would occur.

The expression value from the 2 duplicate experiments are shown in Table 1. The following PCR probe assay synthesized at (Integrated DNA technologies (IDT)) were used:

TARDBP:
Primer 1:
CAGCTCATCCTCAGTCATGTC,
Primer 2:
GATGGTGTGACTGCAAACTTC,
Probe:
/5Cy5/CAGCGCCCCACAAACACTTTTCT/3IAbRQSp/)
ACTL6B wt (ex4-ex5):
Primer 1:
TCTGAGCCAAACCTGCAC,
Primer 2:
ATCAGCTCTGTCAGCTTCTCC,
Probe:
/5HEX/CGAGGCTCC/ZEN/GTGGAACACACG/3IABKFQ/)

The following CY5.5 labelled HPRT1 probe was purchased from BioRad: dHsaCPE13136107.

TABLE 1
Production of TDP43 and ACTL6B WT following
exposure to oligonucleotides
Data shown in Table 2 was normalized to the expression of the house
keeping gene HPRT1, and finally normalized to the average expression
value of the control wells (PBS) that didn't receive any TDP43
knock-down or CA-repeat ASO. The average expression for all of
the given condition is shown in the last column. KD
(“knockdown”, describes wells that only received treatment
with the gapmer ASO that degrades the TDP43 mRNA)

| Oligo | | | Plate A | Plate B | Average |
|---|---|---|---|---|---|
| SEQ | Plate A | Plate B | ACTL6B | ACTL6B | ACTL6B WT |
| ID NO | TDP43 | TDP43 | WT | WT | values |
| PBS | 108.4 | 97.4 | 117.2 | 65.3 | |
| PBS | 110.3 | 99.1 | 95.7 | 74.3 | |
| PBS | 101.2 | 105.5 | 122.6 | 127.8 | |
| PBS | 80.1 | 98.0 | 64.4 | 132.6 | |
| PBS | 101.8 | 94.9 | 111.8 | 81.7 | |
| PBS | 82.4 | 83.7 | 75.3 | 115.4 | |
| PBS | 100.6 | 106.6 | 106.4 | 63.2 | |
| PBS | 115.2 | 114.8 | 106.5 | 139.7 | 100 |
| KD | 4.0 | 5.5 | 1.6 | 0.0 | |
| KD | 5.7 | 5.9 | 0.4 | 0.6 | |
| KD | 5.1 | 6.3 | 1.2 | 0.6 | |
| KD | 5.7 | 8.4 | 1.9 | 0.0 | |
| KD | 4.0 | 6.7 | 1.9 | 0.8 | |
| KD | 5.5 | 4.4 | 2.1 | 0.9 | |
| KD | 6.0 | 4.6 | 0.9 | 1.5 | |
| KD | 7.4 | 5.6 | 1.1 | 1.7 | |
| KD | 5.4 | 5.3 | 2.3 | 1.4 | |
| KD | 5.4 | 9.0 | 1.4 | 1.3 | |
| KD | 6.9 | 5.8 | 0.4 | 1.1 | |
| KD | 4.5 | 5.3 | 2.3 | 0.6 | |
| KD | 4.9 | 3.1 | 0.8 | 1.9 | |
| KD | 5.8 | 5.2 | 1.6 | 0.0 | |
| KD | 5.8 | 5.0 | 0.0 | 0.5 | |
| KD | 5.4 | 7.3 | 0.9 | 0.5 | |
| KD | 5.3 | 7.7 | 1.6 | 0.0 | |
| KD | 6.0 | 6.2 | 0.8 | 1.9 | |
| KD | 6.7 | 4.3 | 0.0 | 1.0 | |
| KD | 3.8 | 4.0 | 0.3 | 0.0 | |
| KD | 5.7 | 3.4 | 0.4 | 0.0 | |
| KD | 5.9 | 4.0 | 0.8 | 0.0 | |
| KD | 2.2 | 6.9 | 0.6 | 0.8 | |
| KD | 5.7 | 5.4 | 0.0 | 1.0 | |
| KD | 4.9 | 4.4 | 1.0 | 0.4 | |
| KD | 4.9 | 6.2 | 1.2 | 0.6 | |
| KD | 5.0 | 6.2 | 1.2 | 0.5 | |
| KD | 6.7 | 4.5 | 0.6 | 2.0 | |
| KD | 8.0 | 4.9 | 1.0 | 1.4 | |
| KD | 5.0 | 4.7 | 0.7 | 0.4 | |
| KD | 5.2 | 4.8 | 1.2 | 0.0 | |
| KD | 4.6 | 2.8 | 1.3 | 0.9 | |
| KD | 5.0 | 5.0 | 1.6 | 0.0 | |
| KD | 9.0 | 4.6 | 2.0 | 1.2 | |
| KD | 7.5 | 6.1 | 0.3 | 0.0 | |
| KD | 7.3 | 5.8 | 1.7 | 0.0 | |
| KD | 7.5 | 7.2 | 2.3 | 1.4 | |
| KD | 4.9 | 3.8 | 0.0 | 0.7 | |
| KD | 3.6 | 5.1 | 0.0 | 0.0 | |
| KD | 4.9 | 5.8 | 0.3 | 0.5 | |
| KD | 5.1 | 5.8 | 1.1 | 0.9 | 0.9 |
| SEQ ID 98 | 3.6 | 4.3 | 0.8 | 1.1 | 0.9 |
| SEQ ID 99 | 4.2 | 2.7 | 2.4 | 1.1 | 1.8 |
| SEQ ID | 3.5 | 2.7 | 5.3 | 5.7 | 5.5 |
| 100 | | | | | |
| SEQ ID | 3.3 | 1.8 | 4.7 | 3.2 | 4.0 |
| 101 | | | | | |
| SEQ ID | 2.1 | 4.1 | 1.9 | 0.8 | 1.4 |
| 102 | | | | | |
| SEQ ID | 3.3 | 2.9 | 1.3 | 0.0 | 0.7 |
| 103 | | | | | |
| SEQ ID | 5.5 | 3.6 | 0.6 | 0.9 | 0.8 |
| 104 | | | | | |
| SEQ ID | 2.7 | 2.5 | 3.4 | 1.3 | 2.3 |
| 105 | | | | | |
| SEQ ID | 2.2 | 2.6 | 0.4 | 1.1 | 0.8 |
| 106 | | | | | |
| SEQ ID | 2.4 | 2.9 | 0.8 | 0.0 | 0.4 |
| 107 | | | | | |
| SEQ ID | 1.9 | 5.3 | 0.7 | 0.9 | 0.8 |
| 108 | | | | | |
| SEQ ID | 3.7 | 1.8 | 2.9 | 3.1 | 3.0 |
| 109 | | | | | |
| SEQ ID | 2.5 | 2.4 | 2.5 | 4.0 | 3.3 |
| 110 | | | | | |
| SEQ ID | 1.2 | 2.5 | 2.1 | 4.8 | 3.5 |
| 111 | | | | | |
| SEQ ID | 2.4 | 2.5 | 2.6 | 8.5 | 5.6 |
| 112 | | | | | |
| SEQ ID | 2.1 | 2.7 | 2.1 | 2.6 | 2.4 |
| 113 | | | | | |
| SEQ ID | 3.3 | 3.9 | 1.1 | 2.0 | 1.5 |
| 114 | | | | | |
| SEQ ID | 2.9 | 2.5 | 4.4 | 3.3 | 3.9 |
| 115 | | | | | |
| SEQ ID | 2.6 | 3.4 | 8.3 | 9.8 | 9.1 |
| 116 | | | | | |
| SEQ ID | 6.0 | 5.5 | 15.7 | 11.9 | 13.8 |
| 117 | | | | | |
| SEQ ID | 5.3 | 2.9 | 27.3 | 23.7 | 25.5 |
| 118 | | | | | |
| SEQ ID | 2.9 | 2.2 | 23.3 | 18.2 | 20.7 |
| 119 | | | | | |
| SEQ ID | 13.3 | 3.8 | 39.0 | 52.5 | 45.7 |
| 120 | | | | | |
| SEQ ID | 13.4 | 3.0 | 38.7 | 52.9 | 45.8 |
| 121 | | | | | |
| SEQ ID | 3.2 | 2.6 | 30.7 | 53.8 | 42.3 |
| 122 | | | | | |
| SEQ ID | 4.4 | 4.3 | 52.8 | 69.9 | 61.4 |
| 123 | | | | | |
| SEQ ID | 3.5 | 1.5 | 54.3 | 56.4 | 55.4 |
| 124 | | | | | |
| SEQ ID | 2.0 | 3.8 | 46.5 | 66.9 | 56.7 |
| 125 | | | | | |
| SEQ ID | 3.8 | 4.4 | 21.4 | 22.4 | 21.9 |
| 126 | | | | | |
| SEQ ID | 3.2 | 3.0 | 13.1 | 12.1 | 12.6 |
| 127 | | | | | |
| SEQ ID | 2.3 | 3.0 | 9.9 | 7.9 | 8.9 |
| 128 | | | | | |
| SEQ ID | 5.8 | 4.1 | 17.5 | 5.4 | 11.5 |
| 129 | | | | | |
| SEQ ID | 3.5 | 5.3 | 25.9 | 39.4 | 32.7 |
| 130 | | | | | |
| SEQ ID | 3.2 | 3.1 | 28.0 | 37.0 | 32.5 |
| 131 | | | | | |
| SEQ ID | 3.8 | 3.4 | 19.5 | 19.2 | 19.3 |
| 132 | | | | | |
| SEQ ID | 1.7 | 2.6 | 14.8 | 12.0 | 13.4 |
| 133 | | | | | |
| SEQ ID | 5.3 | 2.3 | 9.2 | 4.4 | 6.8 |
| 134 | | | | | |
| SEQ ID | 4.2 | 3.0 | 16.5 | 23.5 | 20.0 |
| 135 | | | | | |
| SEQ ID | 5.7 | 1.6 | 17.4 | 8.2 | 12.8 |
| 136 | | | | | |
| SEQ ID | 3.7 | 2.6 | 22.0 | 25.1 | 23.6 |
| 137 | | | | | |
| SEQ ID | 2.5 | 2.8 | 22.1 | 32.4 | 27.2 |
| 138 | | | | | |
| SEQ ID | 1.9 | 5.0 | 56.2 | 61.7 | 58.9 |
| 139 | | | | | |
| SEQ ID | 1.7 | 1.9 | 25.4 | 27.4 | 26.4 |
| 140 | | | | | |
| SEQ ID | 3.3 | 1.5 | 2.6 | 3.9 | 3.3 |
| 141 | | | | | |
| SEQ ID | 5.8 | 4.6 | 4.1 | 3.2 | 3.7 |
| 142 | | | | | |
| SEQ ID | 3.3 | 3.4 | 27.0 | 12.7 | 19.9 |
| 143 | | | | | |
| SEQ ID | 3.1 | 1.5 | 29.9 | 32.6 | 31.2 |
| 144 | | | | | |
| SEQ ID | 4.2 | 2.7 | 30.4 | 23.2 | 26.8 |
| 145 | | | | | |
| SEQ ID | 2.0 | 4.3 | 13.5 | 30.7 | 22.1 |
| 146 | | | | | |
| SEQ ID | 2.3 | 3.0 | 25.8 | 31.8 | 28.8 |
| 147 | | | | | |
| SEQ ID | 3.5 | 4.7 | 10.6 | 15.7 | 13.1 |
| 148 | | | | | |
| SEQ ID | 2.8 | 1.9 | 2.2 | 8.9 | 5.5 |
| 149 | | | | | |
| SEQ ID | 2.0 | 2.9 | 5.6 | 22.6 | 14.1 |
| 150 | | | | | |
| SEQ ID | 3.1 | 2.2 | 2.3 | 6.2 | 4.3 |
| 151 | | | | | |
| SEQ ID | 1.3 | | 6.0 | | 6.0 |
| 152 | | | | | |
| SEQ ID | 2.9 | 2.9 | 6.2 | 8.5 | 7.4 |
| 153 | | | | | |
| SEQ ID | 2.7 | 2.7 | 2.4 | 3.9 | 3.1 |
| 154 | | | | | |
| SEQ ID | 2.2 | 5.0 | 10.6 | 7.4 | 9.0 |
| 155 | | | | | |
| SEQ ID | 3.3 | 1.9 | 11.1 | 34.5 | 22.8 |
| 156 | | | | | |
| SEQ ID | 3.5 | 3.5 | 8.4 | 5.0 | 6.7 |
| 157 | | | | | |
| SEQ ID | 3.9 | 3.0 | 14.1 | 17.9 | 16.0 |
| 158 | | | | | |
| SEQ ID | 3.0 | 3.8 | 19.6 | 18.5 | 19.1 |
| 159 | | | | | |
| SEQ ID | 2.6 | 2.4 | 21.3 | 19.9 | 20.6 |
| 194 | | | | | |
| SEQ ID | 3.8 | 3.2 | 14.9 | 18.7 | 16.8 |
| 160 | | | | | |
| SEQ ID | 3.1 | 1.5 | 13.8 | 10.1 | 11.9 |
| 161 | | | | | |
| SEQ ID | 3.5 | 3.9 | 25.7 | 22.7 | 24.2 |
| 162 | | | | | |
| SEQ ID | 3.2 | 5.0 | 16.3 | | 16.3 |
| 163 | | | | | |
| SEQ ID | | 2.5 | | 31.6 | 31.6 |
| 164 | | | | | |
| SEQ ID | 6.0 | 3.0 | 18.5 | 25.9 | 22.2 |
| 165 | | | | | |
| SEQ ID | 3.9 | 4.6 | 25.7 | 20.7 | 23.2 |
| 166 | | | | | |
| SEQ ID | 2.9 | 4.0 | 7.2 | 9.1 | 8.1 |
| 167 | | | | | |
| SEQ ID | 4.1 | 2.3 | 6.7 | 4.3 | 5.5 |
| 168 | | | | | |
| SEQ ID | 3.6 | 5.1 | 2.1 | 1.1 | 1.6 |
| 169 | | | | | |
| SEQ ID | 3.0 | 2.0 | 2.1 | 3.0 | 2.6 |
| 170 | | | | | |
| SEQ ID | 1.7 | 2.8 | 1.4 | 6.4 | 3.9 |
| 171 | | | | | |
| SEQ ID | 8.4 | 2.7 | 1.9 | 2.9 | 2.4 |
| 172 | | | | | |
| SEQ ID | 3.2 | 3.8 | 1.5 | 1.5 | 1.5 |
| 173 | | | | | |
| SEQ ID | 4.7 | 2.6 | 0.9 | 1.6 | 1.2 |
| 195 | | | | | |
| SEQ ID | 3.0 | 2.8 | 0.5 | 1.8 | 1.2 |
| 174 | | | | | |
| SEQ ID | 3.8 | 3.5 | 0.4 | 0.0 | 0.2 |
| 175 | | | | | |
| SEQ ID | 3.9 | 3.3 | 1.4 | 0.5 | 0.9 |
| 176 | | | | | |
| SEQ ID | 3.4 | 3.4 | 2.8 | 2.3 | 2.6 |
| 177 | | | | | |
| SEQ ID | 5.2 | 4.4 | 0.0 | 1.7 | 0.8 |
| 178 | | | | | |
| SEQ ID | 5.4 | 5.9 | 10.9 | 21.2 | 16.0 |
| 179 | | | | | |
| SEQ ID | 4.7 | 1.9 | 14.1 | 16.4 | 15.2 |
| 180 | | | | | |
| SEQ ID | 4.3 | 4.9 | 3.4 | 9.2 | 6.3 |
| 181 | | | | | |
| SEQ ID | 2.6 | 4.9 | 3.5 | 3.0 | 3.3 |
| 196 | | | | | |
| SEQ ID | 3.4 | 4.8 | 0.4 | 1.7 | 1.1 |
| 182 | | | | | |
| SEQ ID | 4.5 | 3.6 | 1.5 | 1.1 | 1.3 |
| 183 | | | | | |
| SEQ ID | 4.9 | 3.6 | 0.4 | 0.0 | 0.2 |
| 184 | | | | | |
| SEQ ID | 3.4 | | 0.0 | | 0.0 |
| 185 | | | | | |
| SEQ ID | 4.7 | 4.5 | 1.3 | 0.6 | 0.9 |
| 197 | | | | | |
| SEQ ID | 7.1 | 4.1 | 9.5 | 7.9 | 8.7 |
| 186 | | | | | |
| SEQ ID | 3.6 | 6.7 | 10.0 | 3.9 | 7.0 |
| 187 | | | | | |
| SEQ ID | 2.7 | 2.5 | 4.8 | 4.2 | 4.5 |
| 188 | | | | | |
| SEQ ID | 2.9 | 5.2 | 3.8 | 2.4 | 3.1 |
| 189 | | | | | |

COMPOUND TABLE

| Natural | Oligo SEQ | | Target | Target |
|---|---|---|---|---|
| analog sequence | ID NO | HELM | sequence | SEQ ID NO |
| 5′- | SEQ ID 98 | RNA1{[MOE](A)[sP].[MOE](T)[sP]. | GAGACAGCATCTCAC | SEQ ID 6 |
| ATAGTGAGATGCTGTCTC- | | [MOE](A)[sP].[MOE](G)[sP]. | TAT | |
| 3′ | | [MOE](T)[sP].[MOE](G)[sP].[MOE] | | |
| | | (A)[sP].[MOE](G)[sP].[MOE](A) | | |
| | | [sP].[MOE](T)[sP].[MOE](G)[sP] | | |
| | | .[MOE]([5meC])[sP].[MOE](T)[sP] | | |
| | | .[MOE](G)[sP].[MOE](T)[sP]. | | |
| | | [MOE]([5meC])[sP].[MOE](T)[sP] | | |
| | | .[MOE]([5meC])}$$$$V2.0 | | |
| 5′- | SEQ ID 99 | RNA1{[MOE](A)[sP].[MOE]([5meC]) | GACAGCATCTCACTA | SEQ ID 7 |
| ACATAGTGAGATGCTGTC- | | [sP].[MOE](A)[sP].[MOE](T) | TGT | |
| 3′ | | [SP].[MOE](A)[sP].[MOE](G)[sP]. | | |
| | | [MOE](T)[sP].[MOE](G)[sP].[MOE] | | |
| | | (A)[sP].[MOE](G)[sP].[MOE](A) | | |
| | | [sP].[MOE](T)[sP].[MOE](G)[sP] | | |
| | | .[MOE]([5meC])[sP].[MOE](T) | | |
| | | [sP].[MOE](G)[sP].[MOE](T)[sP]. | | |
| | | [MOE]([5meC])}$$$$V2.0 | | |
| 5′- | SEQ ID 100 | RNA1{[MOE]([5meC])[sP].[MOE] | CAGCATCTCACTATG | SEQ ID 8 |
| CAACATAGTGAGATGCTG- | | (A)[sP].[MOE](A)[sP].[MOE] | TTG | |
| 3′ | | ([5meC])[sP].[MOE](A)[sP].[MOE] | | |
| | | (T)[sP].[MOE](A)[sP].[MOE](G) | | |
| | | [sP].[MOE](T)[sP].[MOE](G)[sP]. | | |
| | | [MOE](A)[sP].[MOE](G)[sP].[MOE] | | |
| | | (A)[P].[MOE](T)[sP].[MOE](G) | | |
| | | [sP].[MOE]([5meC])[sP].[MOE](T) | | |
| | | [sP].[MOE](G)}$$$$V2.0 | | |
| 5′- | SEQ ID 101 | RNA1{[MOE](G)[P].[MOE](A)[sP] | GCATCTCACTATGTT | SEQ ID 9 |
| GACAACATAGTGAGATGC- | | .[MOE]([5meC])[sP].[MOE](A) | GTC | |
| 3′ | | [sP].[MOE](A)[sP].[MOE]([5meC] | | |
| | | .[MOE](A)[sP].[MOE](G)[sP]. | | |
| | | [MOE](T)[sP].[MOE](G)[sP].[MOE] | | |
| | | (A)[sP].[MOE](G)[sP].[MOE](A) | | |
| | | [sP].[MOE](T)[sP].[MOE](G)[sP]. | | |
| | | [MOE]([5meC])}$$$$V2.0 | | |
| 5′- | SEQ ID 102 | RNA1{[MOE](T)[sP].[MOE](G)[s | ATCTCACTATGTTGT | SEQ ID 10 |
| TGGACAACATAGTGAGAT- | | P].[MOE](G)[sP].[MOE](A)[sP].[ | CCA | |
| 3′ | | MOE]([5meC])[sP].[MOE](A)[sP] | | |
| | | [MOE](A)[sP].[MOE]([5meC])[s | | |
| | | P].[MOE](A)[sP].[MOE](T)[sP].[ | | |
| | | MOE](A)[sP].[MOE](G)[sP].[MO | | |
| | | E](T)[sP].[MOE](G)[sP].[MOE](A | | |
| | | )[sP].[MOE](G)[sP].[MOE](A)[sP] | | |
| | | .[MOE](T)}$$$$V2.0 | | |
| 5′- | SEQ ID 103 | RNA1{[MOE]([5meC])[sP].[MOE | CTCACTATGTTGTCC | SEQ ID 11 |
| CCTGGACAACATAGTGAG- | | ]([5meC])[sP].[MOE](T)[sP].[MO | AGG | |
| 3′ | | E](G)[sP].[MOE](G)[sP].[MOE](A | | |
| | | )[sP].[MOE]([5meC])[sP].[MOE]( | | |
| | | A)[sP].[MOE](A)[sP].[MOE]([5m | | |
| | | eC])[sP].[MOE](A)[sP].[MOE](T)[ | | |
| | | SP].[MOE](A)[sP].[MOE](G)[sP].[ | | |
| | | MOE](T)[sP].[MOE](G)[sP].[MO | | |
| | | E](A)[sP].[MOE](G)}$$$$V2.0 | | |
| 5′- | SEQ ID 104 | RNA1{[MOE](A)[sP].[MOE](G)[s | CACTATGTTGTCCAG | SEQ ID 12 |
| AGCCTGGACAACATAGTG- | | P].[MOE]([5meC])[sP].[MOE]([5 | GCT | |
| 3′ | | meC])[sP].[MOE](T)[sP].[MOE]( | | |
| | | G)[sP].[MOE](G)[sP].[MOE](A)[s | | |
| | | P].[MOE]([5meC])[sP].[MOE](A) | | |
| | | [sP].[MOE](A)[sP].[MOE]([5meC | | |
| | | ])[sP].[MOE](A)[sP].[MOE](T)[SP | | |
| | | ].[MOE](A)[sP].[MOE](G)[sP].[M | | |
| | | OE](T)[sP].[MOE](G)}$$$$V2.0 | | |
| 5′- | SEQ ID 105 | RNA1{[MOE]([5meC])[sP].[MOE | CTATGTTGTCCAGGC | SEQ ID 13 |
| CTAGCCTGGACAACATAG- | | ](T)[sP].[MOE](A)[sP].[MOE](G)[ | TAG | |
| 3′ | | sP].[MOE]([5meC])[sP].[MOE]([ | | |
| | | 5meC])[sP].[MOE](T)[sP].[MOE] | | |
| | | (G)[sP].[MOE](G)[sP].[MOE](A)[ | | |
| | | SP].[MOE]([5meC])[sP].[MOE](A | | |
| | | )[sP].[MOE](A)[sP].[MOE]([5me | | |
| | | C])[sP].[MOE](A)[sP].[MOE](T)[s | | |
| | | P].[MOE](A)[sP].[MOE](G)}$$$$ | | |
| | | V2.0 | | |
| 5′- | SEQ ID 106 | RNA1{[MOE](A)[sP].[MOE](A)[s | ATGTTGTCCAGGCTA | SEQ ID 14 |
| AACTAGCCTGGACAACAT- | | P].[MOE]([5meC])[sP].[MOE](T)[ | GTT | |
| 3′ | | SP].[MOE](A)[sP].[MOE](G)[sP].[ | | |
| | | MOE]([5meC])[sP].[MOE]([5me | | |
| | | C])[sP].[MOE](T)[sP].[MOE](G)[s | | |
| | | P].[MOE](G)[sP].[MOE](A)[sP].[ | | |
| | | MOE]([5meC])[sP].[MOE](A)[sP] | | |
| | | .[MOE](A)[sP].[MOE]([5meC])[s | | |
| | | P].[MOE](A)[sP].[MOE](T)}$$$$ | | |
| | | V2.0 | | |
| 5′- | SEQ ID 107 | RNA1{[MOE](G)[sP].[MOE](A)[s | GTTGTCCAGGCTAGT | SEQ ID 15 |
| GAAACTAGCCTGGACAAC- | | P].[MOE](A)[sP].[MOE](A)[sP].[ | TTC | |
| 3′ | | MOE]([5meC])[sP].[MOE](T)[sP] | | |
| | | .[MOE](A)[sP].[MOE](G)[sP].[M | | |
| | | OE]([5meC])[sP].[MOE]([5meC]) | | |
| | | [sP].[MOE](T)[sP].[MOE](G)[sP]. | | |
| | | [MOE](G)[sP].[MOE](A)[sP].[MO | | |
| | | E]([5meC])[sP].[MOE](A)[sP].[M | | |
| | | OE](A)[sP].[MOE]([5meC])}$$$$ | | |
| | | V2.0 | | |
| 5′- | SEQ ID 108 | RNA1{[MOE](T)[sP].[MOE]([5m | TGTCCAGGCTAGTTT | SEQ ID 16 |
| TCGAAACTAGCCTGGACA- | | eC])[sP].[MOE](G)[sP].[MOE](A) | CGA | |
| 3′ | | [sP].[MOE](A)[sP].[MOE](A)[sP]. | | |
| | | [MOE]([5meC])[sP].[MOE](T)[sP | | |
| | | ].[MOE](A)[sP].[MOE](G)[P].[M | | |
| | | OE]([5meC])[sP].[MOE]([5meC]) | | |
| | | [sP].[MOE](T)[sP].[MOE](G)[sP]. | | |
| | | [MOE](G)[sP].[MOE](A)[sP].[MO | | |
| | | E]([5meC])[sP].[MOE](A)}$$$$V | | |
| | | 2.0 | | |
| 5′- | SEQ ID 109 | RNA1{[MOE](G)[sP].[MOE](T)[s | TCCAGGCTAGTTTCG | SEQ ID 17 |
| GTTCGAAACTAGCCTGGA- | | P].[MOE](T)[sP].[MOE]([5meC])[ | AAC | |
| 3′ | | SP].[MOE](G)[sP].[MOE](A)[sP].[ | | |
| | | MOE](A)[sP].[MOE](A)[sP].[MO | | |
| | | E]([5meC])[sP].[MOE](T)[sP].[M | | |
| | | OE](A)[sP].[MOE](G)[sP].[MOE]( | | |
| | | [5meC])[sP].[MOE]([5meC])[sP]. | | |
| | | [MOE](T)[sP].[MOE](G)[sP].[MO | | |
| | | E](G)[sP].[MOE](A)}$$$$V2.0 | | |
| 5′- | SEQ ID 110 | RNA1{[MOE](G)[sP].[MOE](A)[s | CAGGCTAGTTTCGAA | SEQ ID 18 |
| GAGTTCGAAACTAGCCTG- | | P].[MOE](G)[sP].[MOE](T)[sP].[ | CTC | |
| 3′ | | MOE](T)[sP].[MOE]([5meC])[sP] | | |
| | | .[MOE](G)[sP].[MOE](A)[P].[M | | |
| | | OE](A)[sP].[MOE](A)[sP].[MOE]( | | |
| | | [5meC])[sP].[MOE](T)[sP].[MOE | | |
| | | ](A)[sP].[MOE](G)[sP].[MOE]([5 | | |
| | | meC])[sP].[MOE]([5meC])[sP].[ | | |
| | | MOE](T)[sP].[MOE](G)}$$$$V2. | | |
| | | 0 | | |
| 5′- | SEQ ID 111 | RNA1{[MOE](A)[sP].[MOE](G)[s | GGCTAGTTTCGAACT | SEQ ID 19 |
| AGGAGTTCGAAACTAGCC- | | P].[MOE](G)[sP].[MOE](A)[sP].[ | CCT | |
| 3′ | | MOE](G)[sP].[MOE](T)[sP].[MO | | |
| | | E](T)[P].[MOE]([5meC])[sP].[M | | |
| | | OE](G)[sP].[MOE](A)[sP].[MOE]( | | |
| | | A)[sP].[MOE](A)[sP].[MOE]([5m | | |
| | | eC])[sP].[MOE](T)[sP].[MOE](A)[ | | |
| | | sP].[MOE](G)[sP].[MOE]([5meC] | | |
| 5′- | SEQ ID 112 | RNA1{[MOE]([5meC])[sP].[MOE | CTAGTTTCGAACTCC | SEQ ID 20 |
| CCAGGAGTTCGAAACTAG- | | ]([5meC])[sP].[MOE](A)[sP].[MO | TGG | |
| 3′ | | E](G)[sP].[MOE](G)[sP].[MOE](A | | |
| | | )[sP].[MOE](G)[sP].[MOE](T)[sP] | | |
| | | [MOE](T)[sP].[MOE]([5meC])[s | | |
| | | P].[MOE](G)[sP].[MOE](A)[sP].[ | | |
| | | MOE](A)[sP].[MOE](A)[sP].[MO | | |
| | | E]([5meC])[sP].[MOE](T)[sP].[M | | |
| | | OE](A)[sP].[MOE](G)}$$$$V2.0 | | |
| 5′- | SEQ ID 113 | RNA1{[MOE](A)[sP].[MOE]([5m | AGTTTCGAACTCCTG | SEQ ID 21 |
| ACCCAGGAGTTCGAAACT- | | eC])[sP].[MOE]([5meC])[sP].[M | GGT | |
| 3′ | | OE]([5meC])[sP].[MOE](A)[sP].[ | | |
| | | MOE](G)[sP].[MOE](G)[sP].[MO | | |
| | | E](A)[sP].[MOE](G)[sP].[MOE](T | | |
| | | )[sP].[MOE](T)[sP].[MOE]([5me | | |
| | | C])[sP].[MOE](G)[sP].[MOE](A)[s | | |
| | | P].[MOE](A)[sP].[MOE](A)[sP].[ | | |
| | | MOE]([5meC])[sP].[MOE](T)}$$ | | |
| | | $$V2.0 | | |
| 5′- | SEQ ID 114 | RNA1{[MOE](G)[sP].[MOE](A)[s | TTTCGAACTCCTGGG | SEQ ID 22 |
| GAACCCAGGAGTTCGAAA- | | P].[MOE](A)[sP].[MOE]([5meC]) | TTC | |
| 3′ | | [sP].[MOE]([5meC])[sP].[MOE]([ | | |
| | | 5meC])[sP].[MOE](A)[sP].[MOE] | | |
| | | (G)[sP].[MOE](G)[sP].[MOE](A)[ | | |
| | | SP].[MOE](G)[sP].[MOE](T)[sP].[ | | |
| | | MOE](T)[sP].[MOE]([5meC])[sP] | | |
| | | .[MOE](G)[sP].[MOE](A)[sP].[M | | |
| | | OE](A)[sP].[MOE](A)}$$$$V2.0 | | |
| 5′- | SEQ ID 115 | RNA1{[MOE](T)[sP].[MOE](T)[sP | TCGAACTCCTGGGTT | SEQ ID 23 |
| TTGAACCCAGGAGTTCGA- | | ].[MOE](G)[sP].[MOE](A)[sP].[M | CAA | |
| 3′ | | OE](A)[sP].[MOE]([5meC])[sP].[ | | |
| | | MOE]([5meC])[sP].[MOE]([5me | | |
| | | C])[sP].[MOE](A)[sP].[MOE](G)[s | | |
| | | P].[MOE](G)[sP].[MOE](A)[sP].[ | | |
| | | MOE](G)[sP].[MOE](T)[sP].[MO | | |
| | | E](T)[sP].[MOE]([5meC])[sP].[M | | |
| | | OE](G)[sP].[MOE](A)}$$$$V2.0 | | |
| 5′- | SEQ ID 116 | RNA1{[MOE](G)[sP].[MOE]([5m | GAACTCCTGGGTTCA | SEQ ID 24 |
| GCTTGAACCCAGGAGTTC- | | eC])[sP].[MOE](T)[sP].[MOE](T)[ | AGC | |
| 3′ | | SP].[MOE](G)[sP].[MOE](A)[sP].[ | | |
| | | MOE](A)[P].[MOE]([5meC])[sP] | | |
| | | .[MOE]([5meC])[sP].[MOE]([5m | | |
| | | eC])[sP].[MOE](A)[sP].[MOE](G) | | |
| | | [sP].[MOE](G)[sP].[MOE](A)[sP]. | | |
| | | [MOE](G)[sP].[MOE](T)[sP].[MO | | |
| | | E](T)[sP].[MOE]([5meC])}$$$$V | | |
| | | 2.0 | | |
| 5′- | SEQ ID 117 | RNA1{[MOE](T)[sP].[MOE](T)[SP | ACTCCTGGGTTCAAG | SEQ ID 25 |
| TTGCTTGAACCCAGGAGT- | | ].[MOE](G)[sP].[MOE]([5meC])[ | CAA | |
| 3′ | | SP].[MOE](T)[sP].[MOE](T)[sP].[ | | |
| | | MOE](G)[P].[MOE](A)[sP].[MO | | |
| | | E](A)[sP].[MOE]([5meC])[sP].[M | | |
| | | OE]([5meC])[sP].[MOE]([5meC]) | | |
| | | [sP].[MOE](A)[sP].[MOE](G)[sP]. | | |
| | | [MOE](G)[sP].[MOE](A)[sP].[MO | | |
| | | E](G)[sP].[MOE](T)}$$$$V2.0 | | |
| 5′- | SEQ ID 118 | RNA1{[MOE](G)[sP].[MOE](A)[s | TCCTGGGTTCAAGCA | SEQ ID 26 |
| GATTGCTTGAACCCAGGA- | | P].[MOE](T)[sP].[MOE](T)[sP].[ | ATC | |
| 3′ | | MOE](G)[sP].[MOE]([5meC])[sP] | | |
| | | .[MOE](T)[sP].[MOE](T)[sP].[MO | | |
| | | E](G)[sP].[MOE](A)[sP].[MOE](A | | |
| | | )[sP].[MOE]([5meC])[sP].[MOE]( | | |
| | | [5meC])[sP].[MOE]([5meC])[sP]. | | |
| | | [MOE](A)[sP].[MOE](G)[sP].[MO | | |
| | | E](G)[sP].[MOE](A)}$$$$V2.0 | | |
| 5′- | SEQ ID 119 | RNA1{[MOE](A)[sP].[MOE](G)[s | CTGGGTTCAAGCAAT | SEQ ID 27 |
| AGGATTGCTTGAACCCAG- | | P].[MOE](G)[sP].[MOE](A)[sP].[ | CCT | |
| 3′ | | MOE](T)[sP].[MOE](T)[sP].[MOE | | |
| | | ](G)[sP].[MOE]([5meC])[sP].[M | | |
| | | OE](T)[sP].[MOE](T)[sP].[MOE]( | | |
| | | G)[sP].[MOE](A)[sP].[MOE](A)[s | | |
| | | P].[MOE]([5meC])[sP].[MOE]([5 | | |
| | | meC])[sP].[MOE]([5meC])[sP].[ | | |
| | | MOE](A)[sP].[MOE](G)}$$$$V2. | | |
| | | 0 | | |
| 5′- | SEQ ID 120 | RNA1{[MOE](G)[sP].[MOE](G)[s | GGGTTCAAGCAATCC | SEQ ID 28 |
| GGAGGATTGCTTGAACCC- | | P].[MOE](A)[sP].[MOE](G)[sP].[ | TCC | |
| 3′ | | MOE](G)[sP].[MOE](A)[sP].[MO | | |
| | | E](T)[sP].[MOE](T)[sP].[MOE](G) | | |
| | | [sP].[MOE]([5meC])[sP].[MOE]( | | |
| | | T)[sP].[MOE](T)[sP].[MOE](G)[s | | |
| | | P].[MOE](A)[sP].[MOE](A)[sP].[ | | |
| | | MOE]([5meC])[sP].[MOE]([5me | | |
| | | C])[sP].[MOE]([5meC])}$$$$V2. | | |
| | | 0 | | |
| 5′- | SEQ ID 121 | RNA1{[MOE]([5meC])[sP].[MOE | GTTCAAGCAATCCTC | SEQ ID 29 |
| CAGGAGGATTGCTTGAAC- | | ](A)[sP].[MOE](G)[sP].[MOE](G) | CTG | |
| 3′ | | [sP].[MOE](A)[sP].[MOE](G)[sP]. | | |
| | | [MOE](G)[sP].[MOE](A)[sP].[MO | | |
| | | E](T)[sP].[MOE](T)[sP].[MOE](G) | | |
| | | [sP].[MOE]([5meC])[sP].[MOE]( | | |
| | | T)[sP].[MOE](T)[sP].[MOE](G)[s | | |
| | | P].[MOE](A)[sP].[MOE](A)[sP].[ | | |
| | | MOE]([5meC])}$$$$V2.0 | | |
| 5′- | SEQ ID 122 | RNA1{[MOE](G)[sP].[MOE](G)[s | TCAAGCAATCCTCCT | SEQ ID 30 |
| GGCAGGAGGATTGCTTGA- | | P].[MOE]([5meC])[sP].[MOE](A) | GCC | |
| 3′ | | [sP].[MOE](G)[sP].[MOE](G)[sP]. | | |
| | | [MOE](A)[sP].[MOE](G)[sP].[MO | | |
| | | E](G)[sP].[MOE](A)[sP].[MOE](T | | |
| | | )[sP].[MOE](T)[sP].[MOE](G)[sP] | | |
| | | .[MOE]([5meC])[sP].[MOE](T)[s | | |
| | | P].[MOE](T)[sP].[MOE](G)[sP].[ | | |
| | | MOE](A)}$$$$V2.0 | | |
| 5′- | SEQ ID 123 | RNA1{[MOE](G)[sP].[MOE](A)[s | AAGCAATCCTCCTGC | SEQ ID 31 |
| GAGGCAGGAGGATTGCTT- | | P].[MOE](G)[sP].[MOE](G)[sP].[ | CTC | |
| 3′ | | MOE]([5meC])[sP].[MOE](A)[sP] | | |
| | | .[MOE](G)[sP].[MOE](G)[sP].[M | | |
| | | OE](A)[sP].[MOE](G)[sP].[MOE]( | | |
| | | G)[sP].[MOE](A)[sP].[MOE](T)[s | | |
| | | P].[MOE](T)[sP].[MOE](G)[sP].[ | | |
| | | MOE]([5meC])[sP].[MOE](T)[sP] | | |
| | | .[MOE](T)}$$$$V2.0 | | |
| 5′- | SEQ ID 124 | RNA1{[MOE]([5meC])[sP].[MOE | GCAATCCTCCTGCCT | SEQ ID 32 |
| CCGAGGCAGGAGGATTGC- | | ]([5meC])[sP].[MOE](G)[sP].[M | CGG | |
| 3′ | | OE](A)[sP].[MOE](G)[sP].[MOE]( | | |
| | | G)[P].[MOE]([5meC])[sP].[MOE | | |
| | | ](A)[sP].[MOE](G)[sP].[MOE](G) | | |
| | | [sP].[MOE](A)[sP].[MOE](G)[sP]. | | |
| | | [MOE](G)[sP].[MOE](A)[sP].[MO | | |
| | | E](T)[sP].[MOE](T)[sP].[MOE](G) | | |
| | | [sP].[MOE]([5meC])}$$$$V2.0 | | |
| 5′- | SEQ ID 125 | RNA1{[MOE](G)[sP].[MOE](G)[s | AATCCTCCTGCCTCG | SEQ ID 33 |
| GGCCGAGGCAGGAGGATT- | | P].[MOE]([5meC])[sP].[MOE]([5 | GCC | |
| 3′ | | meC])[sP].[MOE](G)[sP].[MOE]( | | |
| | | A)[sP].[MOE](G)[sP].[MOE](G)[s | | |
| | | P].[MOE]([5meC])[sP].[MOE](A) | | |
| | | [sP].[MOE](G)[P].[MOE](G)[sP]. | | |
| | | [MOE](A)[sP].[MOE](G)[sP].[MO | | |
| | | E](G)[sP].[MOE](A)[sP].[MOE](T | | |
| | | )[sP].[MOE](T)}$$$$V2.0 | | |
| 5′- | SEQ ID 126 | RNA1{[MOE](G)[sP].[MOE](A)[s | TCCTCCTGCCTCGGC | SEQ ID 34 |
| GAGGCCGAGGCAGGAGGA- | | P].[MOE](G)[sP].[MOE](G)[sP].[ | CTC | |
| 3′ | | MOE]([5meC])[sP].[MOE]([5me | | |
| | | C])[sP].[MOE](G)[P].[MOE](A)[s | | |
| | | P].[MOE](G)[sP].[MOE](G)[sP].[ | | |
| | | MOE]([5meC])[sP].[MOE](A)[sP] | | |
| | | .[MOE](G)[sP].[MOE](G)[sP].[M | | |
| | | OE](A)[sP].[MOE](G)[sP].[MOE]( | | |
| | | G)[sP].[MOE](A)}$$$$V2.0 | | |
| 5′- | SEQ ID 127 | RNA1{[MOE](G)[sP].[MOE](G)[s | CTCCTGCCTCGGCCT | SEQ ID 35 |
| GGGAGGCCGAGGCAGGAG- | | P].[MOE](G)[sP].[MOE](A)[P].[ | CCC | |
| 3′ | | MOE](G)[sP].[MOE](G)[sP].[MO | | |
| | | E]([5meC])[sP].[MOE]([5meC])[s | | |
| | | P].[MOE](G)[sP].[MOE](A)[sP].[ | | |
| | | MOE](G)[P].[MOE](G)[sP].[MO | | |
| | | E]([5meC])[sP].[MOE](A)[sP].[M | | |
| | | OE](G)[P].[MOE](G)[sP].[MOE]( | | |
| | | A)[sP].[MOE](G)}$$$$V2.0 | | |
| 5′- | SEQ ID 128 | RNA1{[MOE](T)[sP].[MOE](T)[SP | CCTGCCTCGGCCTCC | SEQ ID 36 |
| TTGGGAGGCCGAGGCAGG- | | ].[MOE](G)[sP].[MOE](G)[sP].[M | CAA | |
| 3′ | | OE](G)[sP].[MOE](A)[sP].[MOE]( | | |
| | | G)[sP].[MOE](G)[sP].[MOE]([5m | | |
| | | eC])[sP].[MOE]([5meC])[sP].[M | | |
| | | OE](G)[sP].[MOE](A)[sP].[MOE]( | | |
| | | G)[sP].[MOE](G)[sP].[MOE]([5m | | |
| | | eC])[sP].[MOE](A)[sP].[MOE](G) | | |
| | | [sP].[MOE](G)}$$$$V2.0 | | |
| 5′- | SEQ ID 129 | RNA1{[MOE]([5meC])[sP].[MOE | TGCCTCGGCCTCCCA | SEQ ID 37 |
| CTTTGGGAGGCCGAGGCA- | | ](T)[sP].[MOE](T)[P].[MOE](T)[ | AAG | |
| 3′ | | SP].[MOE](G)[sP].[MOE](G)[sP].[ | | |
| | | MOE](G)[P].[MOE](A)[sP].[MO | | |
| | | E](G)[sP].[MOE](G)[sP].[MOE]([ | | |
| | | 5meC])[sP].[MOE]([5meC])[sP].[ | | |
| | | MOE](G)[sP].[MOE](A)[sP].[MO | | |
| | | E](G)[sP].[MOE](G)[sP].[MOE]([ | | |
| | | 5meC])[sP].[MOE](A)}$$$$V2.0 | | |
| 5′- | SEQ ID 130 | RNA1{[MOE]([5meC])[sP].[MOE | CCTCGGCCTCCCAAA | SEQ ID 38 |
| CACTTTGGGAGGCCGAGG- | | ](A)[sP].[MOE]([5meC])[sP].[MO | GTG | |
| 3′ | | E](T)[sP].[MOE](T)[sP].[MOE](T) | | |
| | | [sP].[MOE](G)[sP].[MOE](G)[sP]. | | |
| | | [MOE](G)[sP].[MOE](A)[sP].[MO | | |
| | | E](G)[sP].[MOE](G)[sP].[MOE]([ | | |
| | | 5meC])[sP].[MOE]([5meC])[sP].[ | | |
| | | MOE](G)[sP].[MOE](A)[sP].[MO | | |
| | | E](G)[sP].[MOE](G)}$$$$V2.0 | | |
| 5′- | SEQ ID 131 | RNA1{[MOE](A)[sP].[MOE](G)[s | TCGGCCTCCCAAAGT | SEQ ID 39 |
| AGCACTTTGGGAGGCCGA- | | P].[MOE]([5meC])[sP].[MOE](A) | GCT | |
| 3′ | | [sP].[MOE]([5meC])[sP].[MOE]( | | |
| | | T)[sP].[MOE](T)[sP].[MOE](T)[SP | | |
| | | ].[MOE](G)[sP].[MOE](G)[sP].[M | | |
| | | OE](G)[sP].[MOE](A)[sP].[MOE]( | | |
| | | G)[sP].[MOE](G)[sP].[MOE]([5m | | |
| | | eC])[sP].[MOE]([5meC])[sP].[M | | |
| | | OE](G)[sP].[MOE](A)}$$$$V2.0 | | |
| 5′- | SEQ ID 132 | RNA1{[MOE]([5meC])[sP].[MOE | GGCCTCCCAAAGTGC | SEQ ID 40 |
| CCAGCACTTTGGGAGGCC- | | ]([5meC])[sP].[MOE](A)[sP].[MO | TGG | |
| 3′ | | E](G)[sP].[MOE]([5meC])[sP].[M | | |
| | | OE](A)[sP].[MOE]([5meC])[sP].[ | | |
| | | MOE](T)[sP].[MOE](T)[sP].[MOE | | |
| | | ](T)[sP].[MOE](G)[sP].[MOE](G)[ | | |
| | | SP].[MOE](G)[sP].[MOE](A)[P].[ | | |
| | | MOE](G)[sP].[MOE](G)[sP].[MO | | |
| | | E]([5meC])[sP].[MOE]([5meC])} | | |
| | | $$$$V2.0 | | |
| 5′- | SEQ ID 133 | RNA1{[MOE](T)[sP].[MOE]([5m | CCTCCCAAAGTGCTG | SEQ ID 41 |
| TCCCAGCACTTTGGGAGG- | | eC])[sP].[MOE]([5meC])[sP].[M | GGA | |
| 3′ | | OE]([5meC])[sP].[MOE](A)[sP].[ | | |
| | | MOE](G)[P].[MOE]([5meC])[sP] | | |
| | | .[MOE](A)[sP].[MOE]([5meC])[s | | |
| | | P].[MOE](T)[P].[MOE](T)[sP].[ | | |
| | | MOE](T)[sP].[MOE](G)[sP].[MO | | |
| | | E](G)[sP].[MOE](G)[sP].[MOE](A | | |
| | | )[sP].[MOE](G)[sP].[MOE](G)}$$ | | |
| | | $$V2.0 | | |
| 5′- | SEQ ID 134 | RNA1{[MOE](A)[sP].[MOE](A)[s | TCCCAAAGTGCTGGG | SEQ ID 42 |
| AATCCCAGCACTTTGGGA- | | P].[MOE](T)[sP].[MOE]([5meC])[ | ATT | |
| 3′ | | sP].[MOE]([5meC])[sP].[MOE]([ | | |
| | | 5meC])[sP].[MOE](A)[sP].[MOE] | | |
| | | (G)[sP].[MOE]([5meC])[sP].[MO | | |
| | | E](A)[sP].[MOE]([5meC])[sP].[M | | |
| | | OE](T)[sP].[MOE](T)[sP].[MOE]( | | |
| | | T)[sP].[MOE](G)[sP].[MOE](G)[s | | |
| | | P].[MOE](G)[sP].[MOE](A)}$$$$ | | |
| | | V2.0 | | |
| 5′- | SEQ ID 135 | RNA1{[MOE](A)[sP].[MOE](T)[s | CCAAAGTGCTGGGAT | SEQ ID 43 |
| ATAATCCCAGCACTTTGG- | | P].[MOE](A)[sP].[MOE](A)[P].[ | TAT | |
| 3′ | | MOE](T)[sP].[MOE]([5meC])[sP] | | |
| | | .[MOE]([5meC])[sP].[MOE]([5m | | |
| | | eC])[sP].[MOE](A)[sP].[MOE](G) | | |
| | | [sP].[MOE]([5meC])[sP].[MOE]( | | |
| | | A)[sP].[MOE]([5meC])[sP].[MOE | | |
| | | ](T)[sP].[MOE](T)[sP].[MOE](T)[ | | |
| | | sP].[MOE](G)[sP].[MOE](G)}$$$ | | |
| | | $V2.0 | | |
| 5′- | SEQ ID 136 | RNA1{[MOE]([5meC])[sP].[MOE | AAAGTGCTGGGATTA | SEQ ID 44 |
| CTATAATCCCAGCACTTT- | | ](T)[sP].[MOE](A)[sP].[MOE](T)[ | TAG | |
| 3′ | | SP].[MOE](A)[sP].[MOE](A)[sP].[ | | |
| | | MOE](T)[sP].[MOE]([5meC])[sP] | | |
| | | .[MOE]([5meC])[sP].[MOE]([5m | | |
| | | eC])[sP].[MOE](A)[sP].[MOE](G) | | |
| | | [sP].[MOE]([5meC])[sP].[MOE]( | | |
| | | A)[sP].[MOE]([5meC])[sP].[MOE | | |
| | | ](T)[sP].[MOE](T)[sP].[MOE](T)} | | |
| | | $$$$V2.0 | | |
| 5′- | SEQ ID 137 | RNA1{[MOE](G)[sP].[MOE]([5m | AGTGCTGGGATTATA | SEQ ID 45 |
| GCCTATAATCCCAGCACT- | | eC])[sP].[MOE]([5meC])[sP].[M | GGC | |
| 3′ | | OE](T)[sP].[MOE](A)[sP].[MOE]( | | |
| | | T)[sP].[MOE](A)[sP].[MOE](A)[s | | |
| | | P].[MOE](T)[sP].[MOE]([5meC])[ | | |
| | | sP].[MOE]([5meC])[sP].[MOE]([ | | |
| | | 5meC])[sP].[MOE](A)[sP].[MOE] | | |
| | | (G)[sP].[MOE]([5meC])[sP].[MO | | |
| | | E](A)[sP].[MOE]([5meC])[sP].[M | | |
| | | OE](T)}$$$$V2.0 | | |
| 5′- | SEQ ID 138 | RNA1{[MOE](A)[sP].[MOE]([5m | TGCTGGGATTATAGG | SEQ ID 46 |
| ACGCCTATAATCCCAGCA- | | eC])[sP].[MOE](G)[sP].[MOE]([5 | CGT | |
| 3′ | | meC])[sP].[MOE]([5meC])[sP].[ | | |
| | | MOE](T)[sP].[MOE](A)[sP].[MO | | |
| | | E](T)[sP].[MOE](A)[sP].[MOE](A) | | |
| | | [sP].[MOE](T)[sP].[MOE]([5meC | | |
| | | ])[sP].[MOE]([5meC])[sP].[MOE] | | |
| | | ([5meC])[sP].[MOE](A)[sP].[MO | | |
| | | E](G)[sP].[MOE]([5meC])[sP].[M | | |
| | | OE](A)}$$$$V2.0 | | |
| 5′- | SEQ ID 139 | RNA1{[MOE](T)[sP].[MOE](T)[SP | CTGGGATTATAGGCG | SEQ ID 47 |
| TTACGCCTATAATCCCAG- | | ].[MOE](A)[P].[MOE]([5meC])[s | TAA | |
| 3′ | | P].[MOE](G)[sP].[MOE]([5meC]) | | |
| | | [sP].[MOE]([5meC])[sP].[MOE]( | | |
| | | T)[sP].[MOE](A)[sP].[MOE](T)[s | | |
| | | P].[MOE](A)[sP].[MOE](A)[sP].[ | | |
| | | MOE](T)[sP].[MOE]([5meC])[sP] | | |
| | | .[MOE]([5meC])[sP].[MOE]([5m | | |
| | | eC])[sP].[MOE](A)[sP].[MOE](G) | | |
| | | }$$$$V2.0 | | |
| 5′- | SEQ ID 140 | RNA1{[MOE](G)[sP].[MOE]([5m | GGGATTATAGGCGTA | SEQ ID 48 |
| GCTTACGCCTATAATCCC- | | eC])[sP].[MOE](T)[sP].[MOE](T)[ | AGC | |
| 3′ | | SP].[MOE](A)[sP].[MOE]([5meC] | | |
| | | )[sP].[MOE](G)[sP].[MOE]([5me | | |
| | | C])[sP].[MOE]([5meC])[sP].[MO | | |
| | | E](T)[sP].[MOE](A)[sP].[MOE](T) | | |
| | | [sP].[MOE](A)[sP].[MOE](A)[sP]. | | |
| | | [MOE](T)[sP].[MOE]([5meC])[sP | | |
| | | ].[MOE]([5meC])[sP].[MOE]([5m | | |
| | | eC])}$$$$V2.0 | | |
| 5′- | SEQ ID 141 | RNA1{[MOE](T)[sP].[MOE](G)[s | GATTATAGGCGTAAG | SEQ ID 49 |
| TGGCTTACGCCTATAATC- | | P].[MOE](G)[sP].[MOE]([5meC]) | CCA | |
| 3′ | | [sP].[MOE](T)[sP].[MOE](T)[sP].[ | | |
| | | MOE](A)[sP].[MOE]([5meC])[sP] | | |
| | | .[MOE](G)[sP].[MOE]([5meC])[s | | |
| | | P].[MOE]([5meC])[sP].[MOE](T)[ | | |
| | | SP].[MOE](A)[sP].[MOE](T)[sP].[ | | |
| | | MOE](A)[sP].[MOE](A)[sP].[MO | | |
| | | E](T)[sP].[MOE]([5meC])}$$$$V | | |
| | | 2.0 | | |
| 5′- | SEQ ID 142 | RNA1{[MOE](G)[sP].[MOE](G)[s | TTATAGGCGTAAGCC | SEQ ID 50 |
| GGTGGCTTACGCCTATAA- | | P].[MOE](T)[sP].[MOE](G)[sP].[ | ACC | |
| 3′ | | MOE](G)[sP].[MOE]([5meC])[sP] | | |
| | | .[MOE](T)[sP].[MOE](T)[sP].[MO | | |
| | | E](A)[sP].[MOE]([5meC])[sP].[M | | |
| | | OE](G)[sP].[MOE]([5meC])[sP].[ | | |
| | | MOE]([5meC])[sP].[MOE](T)[sP] | | |
| | | .[MOE](A)[sP].[MOE](T)[sP].[M | | |
| | | OE](A)[sP].[MOE](A)}$$$$V2.0 | | |
| 5′- | SEQ ID 143 | RNA1{[MOE](A)[sP].[MOE](G)[s | ATAGGCGTAAGCCAC | SEQ ID 51 |
| AGGGTGGCTTACGCCTAT- | | P].[MOE](G)[sP].[MOE](G)[sP].[ | CCT | |
| 3′ | | MOE](T)[sP].[MOE](G)[sP].[MO | | |
| | | E](G)[sP].[MOE]([5meC])[sP].[M | | |
| | | OE](T)[sP].[MOE](T)[sP].[MOE]( | | |
| | | A)[sP].[MOE]([5meC])[sP].[MOE | | |
| | | ](G)[sP].[MOE]([5meC])[sP].[M | | |
| | | OE]([5meC])[sP].[MOE](T)[sP].[ | | |
| | | 0 | | |
| 5′- | SEQ ID 144 | RNA1{[MOE](A)[sP].[MOE]([5m | AGGCGTAAGCCACCC | SEQ ID 52 |
| ACAGGGTGGCTTACGCCT- | | eC])[sP].[MOE](A)[sP].[MOE](G) | TGT | |
| 3′ | | [sP].[MOE](G)[sP].[MOE](G)[sP]. | | |
| | | [MOE](T)[sP].[MOE](G)[sP].[MO | | |
| | | E](G)[sP].[MOE]([5meC])[sP].[M | | |
| | | OE](T)[sP].[MOE](T)[sP].[MOE]( | | |
| | | A)[sP].[MOE]([5meC])[sP].[MOE | | |
| | | ](G)[sP].[MOE]([5meC])[sP].[M | | |
| | | OE]([5meC])[sP].[MOE](T)}$$$$ | | |
| | | V2.0 | | |
| 5′- | SEQ ID 145 | RNA1{[MOE](A)[sP].[MOE](G)[s | GCGTAAGCCACCCTG | SEQ ID 53 |
| AGACAGGGTGGCTTACGC- | | P].[MOE](A)[sP].[MOE]([5meC]) | TCT | |
| 3′ | | [sP].[MOE](A)[sP].[MOE](G)[sP]. | | |
| | | [MOE](G)[sP].[MOE](G)[sP].[M | | |
| | | OE](T)[P].[MOE](G)[sP].[MOE]( | | |
| | | G)[sP].[MOE]([5meC])[sP].[MOE | | |
| | | ](T)[sP].[MOE](T)[sP].[MOE](A)[ | | |
| | | sP].[MOE]([5meC])[sP].[MOE](G | | |
| | | )[sP].[MOE]([5meC])}$$$$V2.0 | | |
| 5′- | SEQ ID 146 | RNA1{[MOE]([5meC])[sP].[MOE | GTAAGCCACCCTGTC | SEQ ID 54 |
| CCAGACAGGGTGGCTTAC- | | ]([5meC])[sP].[MOE](A)[sP].[MO | TGG | |
| 3′ | | E](G)[sP].[MOE](A)[sP].[MOE]([ | | |
| | | 5meC])[sP].[MOE](A)[sP].[MOE] | | |
| | | (G)[sP].[MOE](G)[sP].[MOE](G)[ | | |
| | | sP].[MOE](T)[sP].[MOE](G)[sP].[ | | |
| | | MOE](G)[P].[MOE]([5meC])[sP] | | |
| | | .[MOE](T)[sP].[MOE](T)[sP].[MO | | |
| | | 2.0 | | |
| 5′- | SEQ ID 147 | RNA1{[MOE](G)[sP].[MOE](G)[s | AAGCCACCCTGTCTG | SEQ ID 55 |
| GGCCAGACAGGGTGGCTT- | | P].[MOE]([5meC])[sP].[MOE]([5 | GCC | |
| 3′ | | meC])[sP].[MOE](A)[sP].[MOE]( | | |
| | | G)[sP].[MOE](A)[sP].[MOE]([5m | | |
| | | eC])[sP].[MOE](A)[sP].[MOE](G) | | |
| | | [sP].[MOE](G)[sP].[MOE](G)[sP]. | | |
| | | [MOE](T)[sP].[MOE](G)[sP].[MO | | |
| | | E](G)[sP].[MOE]([5meC])[sP].[M | | |
| | | OE](T)[sP].[MOE](T)}$$$$V2.0 | | |
| 5′- | SEQ ID 148 | RNA1{[MOE]([5meC])[sP].[MOE | GCCACCCTGTCTGGC | SEQ ID 56 |
| CAGGCCAGACAGGGTGGC- | | ](A)[sP].[MOE](G)[sP].[MOE](G) | CTG | |
| 3′ | | [sP].[MOE]([5meC])[sP].[MOE]([ | | |
| | | 5meC])[sP].[MOE](A)[sP].[MOE] | | |
| | | (G)[sP].[MOE](A)[sP].[MOE]([5 | | |
| | | meC])[sP].[MOE](A)[sP].[MOE]( | | |
| | | G)[sP].[MOE](G)[sP].[MOE](G)[s | | |
| | | P].[MOE](T)[sP].[MOE](G)[sP].[ | | |
| | | $$V2.0 | | |
| 5′- | SEQ ID 149 | RNA1{[MOE]([5meC])[sP].[MOE | CACCCTGTCTGGCCT | SEQ ID 57 |
| CACAGGCCAGACAGGGTG- | | ](A)[sP].[MOE]([5meC])[sP].[MO | GTG | |
| 3′ | | E](A)[sP].[MOE](G)[sP].[MOE](G | | |
| | | )[sP].[MOE]([5meC])[sP].[MOE]( | | |
| | | [5meC])[sP].[MOE](A)[sP].[MOE | | |
| | | ](G)[sP].[MOE](A)[sP].[MOE]([5 | | |
| | | meC])[sP].[MOE](A)[sP].[MOE]( | | |
| | | G)[sP].[MOE](G)[P].[MOE](G)[s | | |
| | | P].[MOE](T)[sP].[MOE](G)}$$$$ | | |
| | | V2.0 | | |
| 5′- | SEQ ID 150 | RNA1{[MOE](T)[sP].[MOE](G)[s | CCCTGTCTGGCCTGT | SEQ ID 58 |
| TGCACAGGCCAGACAGGG- | | P].[MOE]([5meC])[sP].[MOE](A) | GCA | |
| 3′ | | [sP].[MOE]([5meC])[sP].[MOE]( | | |
| | | A)[sP].[MOE](G)[sP].[MOE](G)[s | | |
| | | P].[MOE]([5meC])[sP].[MOE]([5 | | |
| | | meC])[sP].[MOE](A)[sP].[MOE]( | | |
| | | G)[sP].[MOE](A)[sP].[MOE]([5m | | |
| | | eC])[sP].[MOE](A)[sP].[MOE](G) | | |
| | | [sP].[MOE](G)[sP].[MOE](G)}$$ | | |
| | | $$V2.0 | | |
| 5′- | SEQ ID 151 | RNA1{[MOE](T)[P].[MOE](T)[SP | CTGTCTGGCCTGTGC | SEQ ID 59 |
| TTTGCACAGGCCAGACAG- | | ].[MOE](T)[sP].[MOE](G)[sP].[M | AAA | |
| 3′ | | OE]([5meC])[sP].[MOE](A)[sP].[ | | |
| | | MOE]([5meC])[sP].[MOE](A)[sP] | | |
| | | .[MOE](G)[sP].[MOE](G)[sP].[M | | |
| | | OE]([5meC])[sP].[MOE]([5meC]) | | |
| | | [sP].[MOE](A)[sP].[MOE](G)[sP]. | | |
| | | [MOE](A)[sP].[MOE]([5meC])[sP | | |
| | | ].[MOE](A)[sP].[MOE](G)}$$$$V | | |
| | | 2.0 | | |
| 5′- | SEQ ID 152 | RNA1{[MOE]([5meC])[sP].[MOE | GTCTGGCCTGTGCAA | SEQ ID 60 |
| CATTTGCACAGGCCAGAC- | | ](A)[sP].[MOE](T)[sP].[MOE](T)[ | ATG | |
| 3′ | | SP].[MOE](T)[sP].[MOE](G)[sP].[ | | |
| | | MOE]([5meC])[sP].[MOE](A)[sP] | | |
| | | .[MOE]([5meC])[sP].[MOE](A)[s | | |
| | | P].[MOE](G)[sP].[MOE](G)[sP].[ | | |
| | | MOE]([5meC])[sP].[MOE]([5me | | |
| | | C])[sP].[MOE](A)[sP].[MOE](G)[s | | |
| | | P].[MOE](A)[sP].[MOE]([5meC]) | | |
| | | }$$$$V2.0 | | |
| 5′- | SEQ ID 153 | RNA1{[MOE](A)[sP].[MOE](A)[s | CTGGCCTGTGCAAAT | SEQ ID 61 |
| AACATTTGCACAGGCCAG- | | P].[MOE]([5meC])[sP].[MOE](A) | GTT | |
| 3′ | | [sP].[MOE](T)[sP].[MOE](T)[sP].[ | | |
| | | MOE](T)[sP].[MOE](G)[sP].[MO | | |
| | | E]([5meC])[sP].[MOE](A)[sP].[M | | |
| | | OE]([5meC])[sP].[MOE](A)[sP].[ | | |
| | | MOE](G)[sP].[MOE](G)[sP].[MO | | |
| | | E]([5meC])[sP].[MOE]([5meC])[s | | |
| | | P].[MOE](A)[sP].[MOE](G)}$$$$ | | |
| | | V2.0 | | |
| 5′- | SEQ ID 154 | RNA1{[MOE](A)[sP].[MOE](A)[s | GGCCTGTGCAAATGT | SEQ ID 62 |
| AAAACATTTGCACAGGCC- | | P].[MOE](A)[sP].[MOE](A)[sP].[ | TTT | |
| 3′ | | MOE]([5meC])[sP].[MOE](A)[sP] | | |
| | | .[MOE](T)[sP].[MOE](T)[sP].[MO | | |
| | | E](T)[sP].[MOE](G)[sP].[MOE]([5 | | |
| | | meC])[sP].[MOE](A)[sP].[MOE]([ | | |
| | | 5meC])[sP].[MOE](A)[sP].[MOE] | | |
| | | (G)[sP].[MOE](G)[sP].[MOE]([5 | | |
| | | meC])[sP].[MOE]([5meC])}$$$$ | | |
| | | V2.0 | | |
| 5′- | SEQ ID 155 | RNA1{[MOE](T)[sP].[MOE](T)[sP | CCTGTGCAAATGTTT | SEQ ID 63 |
| TTAAAACATTTGCACAGG- | | ].[MOE](A)[sP].[MOE](A)[P].[M | TAA | |
| 3′ | | OE](A)[sP].[MOE](A)[sP].[MOE]( | | |
| | | [5meC])[sP].[MOE](A)[sP].[MOE | | |
| | | ](T)[sP].[MOE](T)[sP].[MOE](T)[ | | |
| | | SP].[MOE](G)[sP].[MOE]([5meC] | | |
| | | )[sP].[MOE](A)[sP].[MOE]([5me | | |
| | | C])[sP].[MOE](A)[P].[MOE](G)[s | | |
| | | P].[MOE](G)}$$$$V2.0 | | |
| 5′- | SEQ ID 156 | RNA1{[MOE](A)[sP].[MOE](T)[s | TGTGCAAATGTTTTA | SEQ ID 64 |
| ATTTAAAACATTTGCACA- | | P].[MOE](T)[sP].[MOE](T)[sP].[ | AAT | |
| 3′ | | MOE](A)[sP].[MOE](A)[sP].[MO | | |
| | | E](A)[sP].[MOE](A)[sP].[MOE]([ | | |
| | | 5meC])[sP].[MOE](A)[sP].[MOE] | | |
| | | (T)[sP].[MOE](T)[sP].[MOE](T)[s | | |
| | | P].[MOE](G)[sP].[MOE]([5meC]) | | |
| | | [sP].[MOE](A)[sP].[MOE]([5meC | | |
| | | ])[sP].[MOE](A)}$$$$V2.0 | | |
| 5′- | SEQ ID 157 | RNA1{[MOE](G)[sP].[MOE]([5m | TGCAAATGTTTTAAA | SEQ ID 65 |
| GCATTTAAAACATTTGCA- | | eC])[sP].[MOE](A)[sP].[MOE](T)[ | TGC | |
| 3′ | | SP].[MOE](T)[sP].[MOE](T)[sP].[ | | |
| | | MOE](A)[sP].[MOE](A)[sP].[MO | | |
| | | E](A)[sP].[MOE](A)[sP].[MOE]([ | | |
| | | 5meC])[sP].[MOE](A)[sP].[MOE] | | |
| | | (T)[sP].[MOE](T)[sP].[MOE](T)[s | | |
| | | P].[MOE](G)[sP].[MOE]([5meC]) | | |
| | | [sP].[MOE](A)}$$$$V2.0 | | |
| 5′- | SEQ ID 158 | RNA1{[MOE](A)[sP].[MOE](T)[s | CAAATGTTTTAAATG | SEQ ID 66 |
| ATGCATTTAAAACATTTG- | | P].[MOE](G)[sP].[MOE]([5meC]) | CAT | |
| 3′ | | [sP].[MOE](A)[P].[MOE](T)[sP]. | | |
| | | [MOE](T)[sP].[MOE](T)[sP].[MO | | |
| | | E](A)[sP].[MOE](A)[sP].[MOE](A | | |
| | | )[sP].[MOE](A)[sP].[MOE]([5me | | |
| | | C])[sP].[MOE](A)[sP].[MOE](T)[s | | |
| | | P].[MOE](T)[sP].[MOE](T)[sP].[ | | |
| | | MOE](G)}$$$$V2.0 | | |
| 5′- | SEQ ID 159 | RNA1{[MOE](A)[sP].[MOE]([5m | AATGTTTTAAATGCA | SEQ ID 67 |
| ACATGCATTTAAAACATT- | | eC])[sP].[MOE](A)[sP].[MOE](T)[ | TGT | |
| 3′ | | sP].[MOE](G)[sP].[MOE]([5meC] | | |
| | | )[sP].[MOE](A)[sP].[MOE](T)[sP] | | |
| | | .[MOE](T)[sP].[MOE](T)[sP].[MO | | |
| | | E](A)[sP].[MOE](A)[sP].[MOE](A | | |
| | | )[sP].[MOE](A)[P].[MOE]([5me | | |
| | | C])[sP].[MOE](A)[sP].[MOE](T)[s | | |
| | | P].[MOE](T)}$$$$V2.0 | | |
| 5′- | SEQ ID 160 | RNA1{[MOE](A)[sP].[MOE]([5m | TTTTAAATGCATGTG | SEQ ID 68 |
| ACACACATGCATTTAAAA- | | eC])[sP].[MOE](A)[sP].[MOE]([5 | TGT | |
| 3′ | | meC])[sP].[MOE](A)[sP].[MOE]([ | | |
| | | 5meC])[sP].[MOE](A)[sP].[MOE] | | |
| | | (T)[sP].[MOE](G)[sP].[MOE]([5m | | |
| | | eC])[sP].[MOE](A)[sP].[MOE](T)[ | | |
| | | SP].[MOE](T)[sP].[MOE](T)[sP].[ | | |
| | | MOE](A)[sP].[MOE](A)[sP].[MO | | |
| | | E](A)[sP].[MOE](A)}$$$$V2.0 | | |
| 5′- | SEQ ID 161 | RNA1{[MOE](A)[sP].[MOE]([5m | TTAAATGCATGTGTG | SEQ ID 69 |
| ACACACACATGCATTTAA- | | eC])[P].[MOE](A)[sP].[MOE]([5 | TGT | |
| 3′ | | meC])[sP].[MOE](A)[sP].[MOE]([ | | |
| | | 5meC])[sP].[MOE](A)[sP].[MOE] | | |
| | | ([5meC])[sP].[MOE](A)[sP].[MO | | |
| | | E](T)[sP].[MOE](G)[sP].[MOE]([5 | | |
| | | meC])[sP].[MOE](A)[sP].[MOE]( | | |
| | | T)[sP].[MOE](T)[sP].[MOE](T)[sP | | |
| | | ].[MOE](A)[sP].[MOE](A)}$$$$V | | |
| | | 2.0 | | |
| 5′- | SEQ ID 162 | RNA1{[MOE](T)[sP].[MOE]([5m | AAATGCATGTGTGTG | SEQ ID 70 |
| TCACACACACATGCATTT- | | eC])[sP].[MOE](A)[sP].[MOE]([5 | TGA | |
| 3′ | | meC])[sP].[MOE](A)[sP].[MOE]([ | | |
| | | 5meC])[sP].[MOE](A)[sP].[MOE] | | |
| | | ([5meC])[sP].[MOE](A)[sP].[MO | | |
| | | E]([5meC])[sP].[MOE](A)[sP].[M | | |
| | | OE](T)[sP].[MOE](G)[P].[MOE]( | | |
| | | [5meC])[sP].[MOE](A)[sP].[MOE | | |
| | | ](T)[sP].[MOE](T)[sP].[MOE](T)} | | |
| | | $$$$V2.0 | | |
| 5′- | SEQ ID 163 | RNA1{[MOE](A)[sP].[MOE](G)[s | ATGCATGTGTGTGTG | SEQ ID 71 |
| AGTCACACACACATGCAT- | | P].[MOE](T)[sP].[MOE]([5meC])[ | ACT | |
| 3′ | | SP].[MOE](A)[sP].[MOE]([5meC] | | |
| | | )[sP].[MOE](A)[sP].[MOE]([5me | | |
| | | C])[sP].[MOE](A)[sP].[MOE]([5m | | |
| | | eC])[sP].[MOE](A)[sP].[MOE]([5 | | |
| | | meC])[sP].[MOE](A)[sP].[MOE]( | | |
| | | T)[sP].[MOE](G)[sP].[MOE]([5m | | |
| | | eC])[sP].[MOE](A)[sP].[MOE](T)} | | |
| | | $$$$V2.0 | | |
| 5′- | SEQ ID 164 | RNA1{[MOE](A)[sP].[MOE]([5m | GCATGTGTGTGTGAC | SEQ ID 72 |
| ACAGTCACACACACATGC- | | eC])[sP].[MOE](A)[sP].[MOE](G) | TGT | |
| 3′ | | [sP].[MOE](T)[sP].[MOE]([5meC | | |
| | | ])[sP].[MOE](A)[sP].[MOE]([5me | | |
| | | C])[sP].[MOE](A)[sP].[MOE]([5m | | |
| | | eC])[sP].[MOE](A)[P].[MOE]([5 | | |
| | | meC])[sP].[MOE](A)[sP].[MOE]([ | | |
| | | 5meC])[sP].[MOE](A)[sP].[MOE] | | |
| | | (T)[sP].[MOE](G)[sP].[MOE]([5m | | |
| | | eC])}$$$$V2.0 | | |
| 5′- | SEQ ID 165 | RNA1{[MOE](T)[sP].[MOE]([5m | ATGTGTGTGTGACTG | SEQ ID 73 |
| TCACAGTCACACACACAT- | | eC])[sP].[MOE](A)[sP].[MOE]([5 | TGA | |
| 3′ | | meC])[sP].[MOE](A)[sP].[MOE]( | | |
| | | G)[P].[MOE](T)[sP].[MOE]([5m | | |
| | | eC])[sP].[MOE](A)[sP].[MOE]([5 | | |
| | | meC])[sP].[MOE](A)[sP].[MOE]([ | | |
| | | 5meC])[sP].[MOE](A)[sP].[MOE] | | |
| | | ([5meC])[sP].[MOE](A)[sP].[MO | | |
| | | E]([5meC])[sP].[MOE](A)[sP].[M | | |
| | | OE](T)}$$$$V2.0 | | |
| 5′- | SEQ ID 166 | RNA1{[MOE](A)[sP].[MOE]([5m | GTGTGTGTGACTGTG | SEQ ID 74 |
| ACTCACAGTCACACACAC- | | eC])[sP].[MOE](T)[sP].[MOE]([5 | AGT | |
| 3′ | | meC])[sP].[MOE](A)[sP].[MOE]([ | | |
| | | 5meC])[sP].[MOE](A)[sP].[MOE] | | |
| | | (G)[sP].[MOE](T)[sP].[MOE]([5m | | |
| | | eC])[sP].[MOE](A)[sP].[MOE]([5 | | |
| | | meC])[sP].[MOE](A)[sP].[MOE]([ | | |
| | | 5meC])[sP].[MOE](A)[sP].[MOE] | | |
| | | ([5meC])[sP].[MOE](A)[sP].[MO | | |
| | | E]([5meC])}$$$$V2.0 | | |
| 5′- | SEQ ID 167 | RNA1{[MOE](A)[sP].[MOE]([5m | GTGTGTGACTGTGAG | SEQ ID 75 |
| ACACTCACAGTCACACAC- | | eC])[sP].[MOE](A)[P].[MOE]([5 | TGT | |
| 3′ | | meC])[sP].[MOE](T)[sP].[MOE]([ | | |
| | | 5meC])[sP].[MOE](A)[sP].[MOE] | | |
| | | ([5meC])[sP].[MOE](A)[sP].[MO | | |
| | | E](G)[sP].[MOE](T)[sP].[MOE]([5 | | |
| | | meC])[sP].[MOE](A)[sP].[MOE]([ | | |
| | | 5meC])[sP].[MOE](A)[sP].[MOE] | | |
| | | ([5meC])[sP].[MOE](A)[sP].[MO | | |
| | | E]([5meC])}$$$$V2.0 | | |
| 5′- | SEQ ID 168 | RNA1{[MOE](T)[sP].[MOE]([5m | GTGTGACTGTGAGTG | SEQ ID 76 |
| TCACACTCACAGTCACAC- | | eC])[sP].[MOE](A)[sP].[MOE]([5 | TGA | |
| 3′ | | meC])[sP].[MOE](A)[sP].[MOE]([ | | |
| | | 5meC])[sP].[MOE](T)[sP].[MOE] | | |
| | | ([5meC])[sP].[MOE](A)[sP].[MO | | |
| | | E]([5meC])[sP].[MOE](A)[sP].[M | | |
| | | OE](G)[sP].[MOE](T)[sP].[MOE]( | | |
| | | [5meC])[sP].[MOE](A)[sP].[MOE | | |
| | | ]([5meC])[sP].[MOE](A)[sP].[MO | | |
| | | E]([5meC])}$$$$V2.0 | | |
| 5′- | SEQ ID 169 | RNA1{[MOE](A)[sP].[MOE](T)[s | GTGACTGTGAGTGTG | SEQ ID 77 |
| ATTCACACTCACAGTCAC- | | P].[MOE](T)[sP].[MOE]([5meC])[ | AAT | |
| 3′ | | SP].[MOE](A)[sP].[MOE]([5meC] | | |
| | | )[sP].[MOE](A)[sP].[MOE]([5me | | |
| | | C])[sP].[MOE](T)[sP].[MOE]([5m | | |
| | | eC])[sP].[MOE](A)[sP].[MOE]([5 | | |
| | | meC])[sP].[MOE](A)[P].[MOE]( | | |
| | | G)[sP].[MOE](T)[sP].[MOE]([5m | | |
| | | eC])[sP].[MOE](A)[sP].[MOE]([5 | | |
| | | meC])}$$$$V2.0 | | |
| 5′- | SEQ ID 170 | RNA1{[MOE](A)[sP].[MOE](T)[s | GACTGTGAGTGTGAA | SEQ ID 78 |
| ATATTCACACTCACAGTC- | | P].[MOE](A)[sP].[MOE](T)[sP].[ | TAT | |
| 3′ | | MOE](T)[sP].[MOE]([5meC])[sP] | | |
| | | .[MOE](A)[sP].[MOE]([5meC])[s | | |
| | | P].[MOE](A)[sP].[MOE]([5meC]) | | |
| | | [sP].[MOE](T)[sP].[MOE]([5meC | | |
| | | ])[sP].[MOE](A)[sP].[MOE]([5me | | |
| | | C])[sP].[MOE](A)[sP].[MOE](G)[s | | |
| | | P].[MOE](T)[sP].[MOE]([5meC])} | | |
| | | $$$$V2.0 | | |
| 5′- | SEQ ID 171 | RNA1{[MOE](A)[sP].[MOE](T)[s | CTGTGAGTGTGAATA | SEQ ID 79 |
| ATATATTCACACTCACAG- | | P].[MOE](A)[sP].[MOE](T)[sP].[ | TAT | |
| 3′ | | MOE](A)[sP].[MOE](T)[sP].[MO | | |
| | | E](T)[sP].[MOE]([5meC])[sP].[M | | |
| | | OE](A)[sP].[MOE]([5meC])[sP].[ | | |
| | | MOE](A)[sP].[MOE]([5meC])[sP] | | |
| | | .[MOE](T)[sP].[MOE]([5meC])[s | | |
| | | P].[MOE](A)[sP].[MOE]([5meC]) | | |
| | | [sP].[MOE](A)[sP].[MOE](G)}$$$ | | |
| | | $V2.0 | | |
| 5′- | SEQ ID 172 | RNA1{[MOE](A)[sP].[MOE]([5m | GTGAGTGTGAATATA | SEQ ID 80 |
| ACATATATTCACACTCAC- | | eC])[sP].[MOE](A)[sP].[MOE](T)[ | TGT | |
| 3′ | | SP].[MOE](A)[sP].[MOE](T)[sP].[ | | |
| | | MOE](A)[sP].[MOE](T)[sP].[MO | | |
| | | E](T)[sP].[MOE]([5meC])[sP].[M | | |
| | | OE](A)[sP].[MOE]([5meC])[sP].[ | | |
| | | MOE](A)[sP].[MOE]([5meC])[sP] | | |
| | | P].[MOE](A)[sP].[MOE]([5meC]) | | |
| | | }$$$$V2.0 | | |
| 5′- | SEQ ID 173 | RNA1{[MOE](A)[sP].[MOE]([5m | GAGTGTGAATATATG | SEQ ID 81 |
| ACACATATATTCACACTC- | | eC])[sP].[MOE](A)[sP].[MOE]([5 | TGT | |
| 3′ | | meC])[sP].[MOE](A)[sP].[MOE]( | | |
| | | T)[sP].[MOE](A)[sP].[MOE](T)[s | | |
| | | P].[MOE](A)[sP].[MOE](T)[sP].[ | | |
| | | MOE](T)[sP].[MOE]([5meC])[sP] | | |
| | | .[MOE](A)[sP].[MOE]([5meC])[s | | |
| | | P].[MOE](A)[sP].[MOE]([5meC]) | | |
| | | [sP].[MOE](T)[sP].[MOE]([5meC | | |
| | | 1)}$$$$V2.0 | | |
| 5′- | SEQ ID 174 | RNA1{[MOE](A)[sP].[MOE]([5m | GTGAATATATGTGTA | SEQ ID 82 |
| ACATACACATATATTCAC- | | eC])[sP].[MOE](A)[sP].[MOE](T)[ | TGT | |
| 3′ | | SP].[MOE](A)[sP].[MOE]([5meC] | | |
| | | )[sP].[MOE](A)[sP].[MOE]([5me | | |
| | | C])[sP].[MOE](A)[sP].[MOE](T)[s | | |
| | | P].[MOE](A)[sP].[MOE](T)[sP].[ | | |
| | | MOE](A)[sP].[MOE](T)[sP].[MO | | |
| | | E](T)[sP].[MOE]([5meC])[sP].[M | | |
| | | OE](A)[sP].[MOE]([5meC])}$$$$ | | |
| | | V2.0 | | |
| 5′- | SEQ ID 175 | RNA1{[MOE](A)[sP].[MOE]([5m | GAATATATGTGTATG | SEQ ID 83 |
| ACACATACACATATATTC- | | eC])[sP].[MOE](A)[sP].[MOE]([5 | TGT | |
| 3′ | | meC])[P].[MOE](A)[sP].[MOE]( | | |
| | | T)[sP].[MOE](A)[sP].[MOE]([5m | | |
| | | eC])[sP].[MOE](A)[sP].[MOE]([5 | | |
| | | meC])[sP].[MOE](A)[P].[MOE]( | | |
| | | T)[sP].[MOE](A)[sP].[MOE](T)[s | | |
| | | P].[MOE](A)[sP].[MOE](T)[sP].[ | | |
| | | MOE](T)[sP].[MOE]([5meC])}$$ | | |
| | | $$V2.0 | | |
| 5′- | SEQ ID 176 | RNA1{[MOE](A)[sP].[MOE]([5m | ATATATGTGTATGTG | SEQ ID 84 |
| ACACACATACACATATAT- | | eC])[sP].[MOE](A)[sP].[MOE]([5 | TGT | |
| 3′ | | meC])[sP].[MOE](A)[sP].[MOE]([ | | |
| | | 5meC])[sP].[MOE](A)[sP].[MOE] | | |
| | | (T)[sP].[MOE](A)[sP].[MOE]([5m | | |
| | | eC])[sP].[MOE](A)[sP].[MOE]([5 | | |
| | | meC])[sP].[MOE](A)[sP].[MOE]( | | |
| | | T)[sP].[MOE](A)[sP].[MOE](T)[s | | |
| | | P].[MOE](A)[sP].[MOE](T)}$$$$ | | |
| | | V2.0 | | |
| 5′- | SEQ ID 177 | RNA1{[MOE](A)[sP].[MOE]([5m | ATATGTGTATGTGTG | SEQ ID 85 |
| ACACACACATACACATAT- | | eC])[sP].[MOE](A)[sP].[MOE]([5 | TGT | |
| 3′ | | meC])[sP].[MOE](A)[sP].[MOE]([ | | |
| | | 5meC])[sP].[MOE](A)[sP].[MOE] | | |
| | | ([5meC])[sP].[MOE](A)[sP].[MO | | |
| | | E](T)[sP].[MOE](A)[sP].[MOE]([5 | | |
| | | meC])[sP].[MOE](A)[sP].[MOE]([ | | |
| | | 5meC])[sP].[MOE](A)[sP].[MOE] | | |
| | | (T)[sP].[MOE](A)[sP].[MOE](T)}$ | | |
| | | $$$V2.0 | | |
| 5′- | SEQ ID 178 | RNA1{[MOE](A)[sP].[MOE]([5m | ATGTGTATGTGTGTG | SEQ ID 86 |
| ACACACACACATACACAT- | | eC])[sP].[MOE](A)[sP].[MOE]([5 | TGT | |
| 3′ | | meC])[sP].[MOE](A)[sP].[MOE]([ | | |
| | | 5meC])[sP].[MOE](A)[sP].[MOE] | | |
| | | ([5meC])[sP].[MOE](A)[sP].[MO | | |
| | | E]([5meC])[sP].[MOE](A)[sP].[M | | |
| | | OE](T)[sP].[MOE](A)[sP].[MOE]( | | |
| | | [5meC])[sP].[MOE](A)[P].[MOE | | |
| | | ]([5meC])[sP].[MOE](A)[sP].[MO | | |
| | | E](T)}$$$$V2.0 | | |
| 5′- | SEQ ID 179 | RNA1{[MOE](T)[sP].[MOE]([5m | GTGTATGTGTGTGTG | SEQ ID 87 |
| TCACACACACACATACAC- | | eC])[sP].[MOE](A)[sP].[MOE]([5 | TGA | |
| 3′ | | meC])[sP].[MOE](A)[sP].[MOE]([ | | |
| | | 5meC])[sP].[MOE](A)[sP].[MOE] | | |
| | | ([5meC])[sP].[MOE](A)[sP].[MO | | |
| | | E]([5meC])[sP].[MOE](A)[sP].[M | | |
| | | OE]([5meC])[sP].[MOE](A)[sP].[ | | |
| | | MOE](T)[sP].[MOE](A)[sP].[MO | | |
| | | E]([5meC])[sP].[MOE](A)[sP].[M | | |
| | | OE]([5meC])}$$$$V2.0 | | |
| 5′- | SEQ ID 180 | RNA1{[MOE](A)[sP].[MOE]([5m | GTATGTGTGTGTGTG | SEQ ID 88 |
| ACTCACACACACACATAC- | | eC])[sP].[MOE](T)[sP].[MOE]([5 | AGT | |
| 3′ | | meC])[sP].[MOE](A)[sP].[MOE]([ | | |
| | | 5meC])[sP].[MOE](A)[sP].[MOE] | | |
| | | ([5meC])[sP].[MOE](A)[sP].[MO | | |
| | | E]([5meC])[sP].[MOE](A)[sP].[M | | |
| | | OE]([5meC])[sP].[MOE](A)[sP].[ | | |
| | | MOE]([5meC])[sP].[MOE](A)[sP] | | |
| | | .[MOE](T)[sP].[MOE](A)[sP].[M | | |
| | | OE]([5meC])}$$$$V2.0 | | |
| 5′- | SEQ ID 181 | RNA1{[MOE](A)[sP].[MOE](A)[s | ATGTGTGTGTGTGAG | SEQ ID 89 |
| AAACTCACACACACACAT- | | P].[MOE](A)[sP].[MOE]([5meC]) | TTT | |
| 3′ | | [sP].[MOE](T)[sP].[MOE]([5meC | | |
| | | ])[sP].[MOE](A)[sP].[MOE]([5me | | |
| | | C])[sP].[MOE](A)[sP].[MOE]([5m | | |
| | | eC])[sP].[MOE](A)[sP].[MOE]([5 | | |
| | | meC])[sP].[MOE](A)[sP].[MOE]([ | | |
| | | 5meC])[sP].[MOE](A)[sP].[MOE] | | |
| | | ([5meC])[sP].[MOE](A)[sP].[MO | | |
| | | E](T)}$$$$V2.0 | | |
| 5′- | SEQ ID 182 | RNA1{[MOE](A)[sP].[MOE]([5m | GTGTGTGTGAGTTTG | SEQ ID 90 |
| ACACAAACTCACACACAC- | | eC])[sP].[MOE](A)[sP].[MOE]([5 | TGT | |
| 3′ | | meC])[sP].[MOE](A)[sP].[MOE]( | | |
| | | A)[sP].[MOE](A)[sP].[MOE]([5m | | |
| | | eC])[sP].[MOE](T)[sP].[MOE]([5 | | |
| | | meC])[sP].[MOE](A)[sP].[MOE]([ | | |
| | | 5meC])[sP].[MOE](A)[sP].[MOE] | | |
| | | ([5meC])[sP].[MOE](A)[sP].[MO | | |
| | | E]([5meC])[sP].[MOE](A)[sP].[M | | |
| | | OE]([5meC])}$$$$V2.0 | | |
| 5′- | SEQ ID 183 | RNA1{[MOE](A)[sP].[MOE]([5m | GTGTGTGAGTTTGTG | SEQ ID 91 |
| ACACACAAACTCACACAC- | | meC])[sP].[MOE](A)[sP].[MOE]([ | TGT | |
| 3′ | | 5meC])[sP].[MOE](A)[sP].[MOE] | | |
| | | (A)[sP].[MOE](A)[sP].[MOE]([5 | | |
| | | meC])[sP].[MOE](T)[sP].[MOE]([ | | |
| | | 5meC])[sP].[MOE](A)[sP].[MOE] | | |
| | | ([5meC])[sP].[MOE](A)[sP].[MO | | |
| | | E]([5meC])[sP].[MOE](A)[sP].[M | | |
| | | OE]([5meC])}$$$$V2.0 | | |
| 5′- | SEQ ID 184 | RNA1{[MOE](T)[sP].[MOE]([5m | GTGTGAGTTTGTGTG | SEQ ID 92 |
| TCACACACAAACTCACAC- | | eC])[sP].[MOE](A)[sP].[MOE]([5 | TGA | |
| 3′ | | meC])[sP].[MOE](A)[sP].[MOE]([ | | |
| | | 5meC])[sP].[MOE](A)[sP].[MOE] | | |
| | | ([5meC])[sP].[MOE](A)[sP].[MO | | |
| | | E](A)[sP].[MOE](A)[sP].[MOE]([ | | |
| | | 5meC])[sP].[MOE](T)[sP].[MOE] | | |
| | | ([5meC])[sP].[MOE](A)[sP].[MO | | |
| | | E]([5meC])[sP].[MOE](A)[sP].[M | | |
| | | OE]([5meC])}$$$$V2.0 | | |
| 5′- | SEQ ID 185 | RNA1{[MOE](A)[sP].[MOE](T)[s | GTGAGTTTGTGTGTG | SEQ ID 93 |
| ATTCACACACAAACTCAC- | | P].[MOE](T)[sP].[MOE]([5meC])[ | AAT | |
| 3′ | | SP].[MOE](A)[sP].[MOE]([5meC] | | |
| | | )[sP].[MOE](A)[sP].[MOE]([5me | | |
| | | C])[sP].[MOE](A)[sP].[MOE]([5m | | |
| | | eC])[sP].[MOE](A)[sP].[MOE](A) | | |
| | | [sP].[MOE](A)[sP].[MOE]([5meC | | |
| | | ])[sP].[MOE](T)[sP].[MOE]([5me | | |
| | | C])[sP].[MOE](A)[sP].[MOE]([5m | | |
| | | eC])}$$$$V2.0 | | |
| 5′- | SEQ ID 186 | RNA1{[MOE](T)[sP].[MOE]([5m | GTTTGTGTGTGAATG | SEQ ID 94 |
| TCACATTCACACACAAAC- | | eC])[sP].[MOE](A)[sP].[MOE]([5 | TGA | |
| 3′ | | meC])[sP].[MOE](A)[sP].[MOE]( | | |
| | | T)[sP].[MOE](T)[sP].[MOE]([5m | | |
| | | eC])[sP].[MOE](A)[sP].[MOE]([5 | | |
| | | meC])[sP].[MOE](A)[sP].[MOE]([ | | |
| | | 5meC])[sP].[MOE](A)[sP].[MOE] | | |
| | | ([5meC])[sP].[MOE](A)[sP].[MO | | |
| | | E](A)[sP].[MOE](A)[sP].[MOE]([ | | |
| | | 5meC])}$$$$V2.0 | | |
| 5′- | SEQ ID 187 | RNA1{[MOE](A)[sP].[MOE]([5m | TTGTGTGTGAATGTG | SEQ ID 95 |
| ACTCACATTCACACACAA- | | eC])[sP].[MOE](T)[sP].[MOE]([5 | AGT | |
| 3′ | | meC])[sP].[MOE](A)[sP].[MOE]([ | | |
| | | 5meC])[sP].[MOE](A)[sP].[MOE] | | |
| | | (T)[sP].[MOE](T)[sP].[MOE]([5m | | |
| | | eC])[sP].[MOE](A)[sP].[MOE]([5 | | |
| | | meC])[sP].[MOE](A)[sP].[MOE]([ | | |
| | | 5meC])[sP].[MOE](A)[sP].[MOE] | | |
| | | ([5meC])[sP].[MOE](A)[sP].[MO | | |
| | | E](A)}$$$$V2.0 | | |
| 5′- | SEQ ID 188 | RNA1{[MOE](A)[sP].[MOE](T)[s | GTGTGTGAATGTGAG | SEQ ID 96 |
| ATACTCACATTCACACAC- | | P].[MOE](A)[sP].[MOE]([5meC]) | TAT | |
| 3′ | | [sP].[MOE](T)[sP].[MOE]([5meC | | |
| | | ])[sP].[MOE](A)[sP].[MOE]([5me | | |
| | | C])[sP].[MOE](A)[sP].[MOE](T)[s | | |
| | | sP].[MOE](A)[sP].[MOE]([5meC] | | |
| | | )[sP].[MOE](A)[sP].[MOE]([5me | | |
| | | C])[sP].[MOE](A)[sP].[MOE]([5m | | |
| | | eC])}$$$$V2.0 | | |
| 5′- | SEQ ID 189 | RNA1{[MOE](A)[sP].[MOE](T)[s | GTGTGAATGTGAGTA | SEQ ID 97 |
| ATATACTCACATTCACAC- | | P].[MOE](A)[sP].[MOE](T)[sP].[ | TAT | |
| 3′ | | MOE](A)[sP].[MOE]([5meC])[sP] | | |
| | | [MOE](T)[sP].[MOE]([5meC])[s | | |
| | | P].[MOE](A)[sP].[MOE]([5meC]) | | |
| | | [sP].[MOE](A)[sP].[MOE](T)[sP]. | | |
| | | [MOE](T)[sP].[MOE]([5meC])[sP | | |
| | | ].[MOE](A)[sP].[MOE]([5meC])[s | | |
| | | P].[MOE](A)[P].[MOE]([5meC]) | | |
| | | }$$$$V2.0 | | |
| 5′- | SEQ ID 194 | RNA1{[MOE](A)[sP].[MOE]([ | TGTTTTAAATGCATG | SEQ ID 190 |
| ACACATGCATTTAAA | | 5meC])[sP].[MOE](A)[sP].[MO | TGT | |
| ACA-3′ | | E]([5meC])[sP].[MOE](A)[sP].[ | | |
| | | MOE](T)[sP].[MOE](G)[sP].[M | | |
| | | OE]([5meC])[sP].[MOE](A)[sP | | |
| | | ].[MOE](T)[sP].[MOE](T)[sP].[ | | |
| | | MOE](T)[sP].[MOE](A)[sP].[M | | |
| | | OE](A)[sP].[MOE](A)[P].[MO | | |
| | | E](A)[sP].[MOE]([5meC])[sP].[ | | |
| | | MOE](A)}$$$$V2.0 | | |
| 5′- | SEQ ID 195 | RNA1{[MOE](A)[sP].[MOE]( | GTGTGAATATATGTG | SEQ ID 190 |
| ATACACATATATTCAC | | T)[sP].[MOE](A)[sP].[MOE]([5 | TAT | |
| AC-3′ | | meC])[sP].[MOE](A)[sP].[MO | | |
| | | E]([5meC])[sP].[MOE](A)[sP].[ | | |
| | | MOE](T)[sP].[MOE](A)[sP].[M | | |
| | | OE](T)[sP].[MOE](A)[sP].[MO | | |
| | | E](T)[sP].[MOE](T)[sP].[MOE] | | |
| | | ([5meC])[sP].[MOE](A)[sP].[M | | |
| | | OE]([5meC])[sP].[MOE](A)[sP | | |
| 5′- | SEQ ID 196 | RNA1{[MOE](A)[sP].[MOE]([ | GTGTGTGTGTGAGTT | SEQ ID 192 |
| ACAAACTCACACACA | | 5meC])[sP].[MOE](A)[sP].[MO | TGT | |
| CAC-3′ | | E](A)[sP].[MOE](A)[sP].[MOE | | |
| | | MOE]([5meC])[sP].[MOE](A)[ | | |
| | | sP].[MOE]([5meC])[sP].[MOE] | | |
| | | (A)[sP].[MOE]([5meC])[sP].[M | | |
| | | OE](A)[sP].[MOE]([5meC])[sP | | |
| | | ].[MOE](A)[sP].[MOE]([5meC] | | |
| | | )[sP].[MOE](A)[sP].[MOE]([5 | | |
| | | meCl)}$$$$V2.0 | | |
| 5′- | SEQ ID 197 | RNA1{[MOE](A)[sP].[MOE]([ | GAGTTTGTGTGTGAA | SEQ ID 193 |
| ACATTCACACACAAA | | 5meC])[sP].[MOE](A)[sP].[MO | TGT | |
| CTC-3′ | | E](T)[sP].[MOE](T)[sP].[MOE] | | |
| | | ([5meC])[sP].[MOE](A)[sP].[M | | |
| | | OE]([5meC])[sP].[MOE](A)[sP | | |
| | | ].[MOE]([5meC])[sP].[MOE](A | | |
| | | )[sP].[MOE]([5meC])[sP].[MO | | |
| | | E](A)[sP].[MOE](A)[sP].[MOE | | |
| | | ](A)[sP].[MOE]([5meC])[sP].[ | | |
| | | MOE](T)[sP].[MOE]([5meC])} | | |
| | | $$$$V2.0 | | |
| 5′- | SEQ ID 198 | RNAI[LR](T)[sP].[LR]([5me | | |
| TCCACACTGAACAAA | | CD)[sP].[dR](C)[sP].[dR](A)[sP | | |
| CC-3′ | | C)[sP].[dR](T)[sP].[dR](G)[sP]. | | |
| | | [dR](A)[sP].[dR](A)[sP].[dR](C | | |
| | | )[sP].[dR](A)[sP].[LR](A)SP].[ | | |
| | | LR]([5meC])}$$$$V2.0 | | |

Helm Annotation Key:
[LR](G) is a beta-D-oxy-LNA guanine nucleoside,
[LR](T) is a beta-D-oxy-LNA thymine nucleoside,
[LR](A) is a beta-D-oxy-LNA adenine nucleoside,
[LR]([5meC] is a beta-D-oxy-LNA 5-methyl cytosine nucleoside,
[MOE](G) is a 2′-O-methoxyethyl-RNA guanine nucleoside,
[MOE](T) 2′-O-methoxyethyl-RNA thymine nucleoside,
[MOE](A) 21-O-methoxyethyl-RNA adenine nucleoside,
[MOE]([5meC] 21-O-methoxyethyl-RNA 5-methyl cytosine nucleoside,
[dR](G) is a DNA guanine nucleoside,
[dR](T) is a DNA thymine nucleoside,
[dR](A) is a DNA adenine nucleoside,
[dR]([C] is a DNA cytosine nucleoside,
[mR](G) is a 2′-O-methyl RNA guanine nucleoside,
[mR](U) is a 2′-O-methyl RNA DNA uracil nucleoside,
[mR](A) is a 2′-O-methyl RNA DNA adenine nucleoside,
[mR]([C] is a 2′-O-methyl RNA DNA cytosine nucleoside.

SEQUENCES
ACTL6B precursor-mRNA
GRCh38.p13, Chromosome 7: 100,643,097-100,656,448 reverse strand
SEQ ID NO: 1
GAGTCCCGCCCCGCCAGGGATCCCGGGAGCTGTCCGGCCGCCTCGGTGCTGA
TCCCGCCACCGCCCACGGGCCGCTAGCAGCGCAGCGGGCACTATGAGCGGG
GGCGTCTACGGCGGAGGTGAGCGAGCCTCGGGCCCCCGGCTCCCGCAGCCC
TGCGTTCCAAACCCGCGCCATCCATCCGAGCTCTGGGCACCCCGGGGCCTGT
CACCTCCGCGCACGAGCGGGTCGAGTCTCAGGCTCGGGTCTCCCGCCACCCC
TGCGCTCGGTCCTCCCCAGACGCCCCGCGCGCACAGGGCCCTACCTTGCAAC
CCACCTCCACCTGGCCCCGGGCCCCTTCCTTTCCCGTCGGGAGGGTGCCCTG
AGCAAGGCCATTGGGCAAGAAGGGACCCTGGCGGTGTGATGCCCCCACCCCC
AGTCCCAGGACCTCGGACTCCAGGCCCAGCTCCCACCGGCCCCAGACTGGTG
TTGAAAGTGGCGAGACTGTGGCTCTGCCCTGACTTTCTCTCGGAGCTACCTAG
CGGGAGAGAGTTCGGGGGAGGTCCCCTTACAGGCCTGTCTCCCCGCACAGAT
GAGGTGGGGGCGCTGGTCTTTGACATTGGCTCCTTCTCAGTCCGCGCTGGGTA
CGCTGGGGAGGACTGTCCCAAGGTGAGCCTTCCAGTCTCCTCTCCAAATCCTA
GGGGCTTCGGACCCTTCTTCAGGCTTTCCAGTGACCCAGGGCTCTCCCAGATC
GCCCAGAAGCTCTGCTGAGCGGGAATATGGGAATAAACCCAGGGGCGGTCTG
AGGACCCTGCTGTGGGGCTGGACAGGGGCAAGAGGGTGACCTCTCTCCCTGC
CAATACTCCCCCAACCCTTCCCCACCAGGCTGACTTCCCCACCACAGTGGGGC
TGCTGGCCGCGGAGGAGGGGGGCGGGCTGGAGCTGGAGGGGGACAAAGAGA
AGAAAGGGAAGATCTTCCACATCGACACCAATGCCCTGCACGTGCCTCGGGAT
GGAGCGGAGGTCATGTCGCCCCTCAAGAATGGCATGAGTAAGGGGCCCCCAC
CCATCACCTCCTGACAGAAAAGGAGCCCGCTCCCCACAATCTGGGTCATAGCA
GCCCCCACTCCCCGCGGAGTCTTCCTTGCAGCCCCAGGGTTCTTCTGGCTGCC
AGGGTTCTTCTCGCTGGGTTCCCTTCCCAGGGGCCCACCAGGTTTCTGGGAAG
GGGGGCGTTCCCCTGGAGCTGGACTCACTGACCCTCCTTTGGGCTTGGCCTTT
CTCCCTCCTTTTCTTCCTGTCCCTGTCCCCTGCCTGCCTTCTTGCGTCTCTGCA
CGCTGCTCTTCTGGCCCCAGTCGAGGACTGGGAGTGCTTCCGAGCCATCCTGG
ATCACACCTACAGCAAACACGTCAAGTCTGAGCCAAACCTGCACCCAGTGCTCA
TGTCCGAGGCTCCGGTGAGTGCCTCCTCCATCCTCATGTCCAACCCTGATCTC
CACTGCACCTCCTTCCCACCATCCACCTCTCCTCCTCTCCCCTCAAGCCACTGC
TCTGAGCCACTTCCCTTCCCTTTTCAGACTCCCCTCACCCATCCCAAACAATTTC
CTTCCTTCTTGGCTAAGTCTCCATCCCTCCTGCTCTCTTCTTGCTCTATGCACTC
TGCTGCCCTTCTCTGTCTCCAAGTCCTTTTTAAATTTTTTTTTTTTGACACAGTCT
CACTCCGTCACCCAGGCTGGAGTGCAATGGTGAGATCTCAGCCTCTGCCTCCC
GACATCAAAGGAATCTCCTGCCTCAGCCTCCTGAGTATCTGGGATTACAGGCAC
CCGCCACCACGCCTGGCTAATTTTTTGTATTTTTAGTAGACAGGGGGTTTCGCC
TTGTTGGCCAGGCTGGTCTCGAACTCCTGACCTCAGGTGATCCACCCGCCTTG
GCCTCCCAAAGTGTTGGGATTACAGGCATGAGGCACCACGCCCAGCCTCCAAG
TCCTTTTTTGACCTTTTCACTTATTATTATTATTATTATTATTATTATTATTATTATT
ATGGTCAACTTTGGTGAGCTGGGGATGTTGGCTCACACCTGTAATCCCAACACT
TTGGGAAGCCAAGGCAGGAGGATCACTTGAGCCCAGGAGTTTGAGACCACCCT
AGGCAATATGGCAAGACCCGTCTCTTTTATTAAAACAATAAAAATTAAAAATTTTT
TGCCGGGTGTGGTGGCTCACGCCTGTAATCCCAGCACTTTGGGAGGCCGAGG
CGGGTGGATCACGAGGTCAAGAGATCAAGACCATCCTGGATAACACGGTGAAA
CCCCATCTCTACTAAAAATACAAAAAATTAGCCAGGCATGGTGGCGGGCACCTG
TAGTCCCAGCTACTCGGGAGGCTGAGGCAGGAGAATGGCGTGAACCCGGGAG
GCGGAGCTTGCAGTGAGCTGAGATCGCACCACTGCACTCCAGCCTGGGCAAC
AGAGCGAGACTCTGTCTCAAAAAAAAAAAAAATTTAAAACCTCATTTAAAAAAAT
GTTCTTACCCACCTCCTTATAAATTCCTCTTTATAAAAGCAACTGTTTTTCTCCTT
TTTAGCTAATGTTGGGGGTATTTTCTTCCATATCTCTAATACTACACTTAAATTGC
TGCCTGTGGAGTGTTTAGTTTTAGGTATTATCGCTGAATTTTCCACTTTAGAAAA
TGATCGCTTAGCTCTCTTTTCCTCCCCAGCCTATGCCACACGTATTTCTTATCAC
CCTCCTTTAAGTACAGTGGGTTTTGTTGTTGTTGTTGTTGTTGTTTGGGGTTTTG
TTTGTTGTTTGTTTTTGAGACTTTGCCTCAAAAACAGGGGGTCTGCTCAGTTTGT
CACCCAGGTTGGATTGCAGTGGCACAATTCTTCAGCCTTGGTCTCCTGGGCTC
AAGTGATCCTCTCATTTCAGTCTCCAGAGTAGCTGGAACTACAGGCATAAGCCA
CCATGCCCAGCTGATTTTTAAATTTTTTTTAGAGACAGGGTTTCATCATGTTGTC
CAGGCTGGTCTTGAATTCCTGGGCTCAAGCAATCTGCCCGCCTCGGCCTCCCA
AAAGGCTGGGATTACAGGCGAGATCCACCACACCCAGCCCAGTTGTATTTTAG
ACTTTAAAAGAAATCAGTATTCAGTATTTACATTCTTTTTAAATTAGAAATAATTTT
AAGAGAATCTTTTGCAGAGATGGGGTCTCATCATATTGCCCAGGCTGGCTTCAA
ACTCCTGTGCTTAAGCGATCCTTCTACTTCAGCTAACAAAGTGCTGGGATTACA
GGCATGAGTCACTGTGCTCAGCCCAATATTTACACTCCTATTACTATGTATGCTA
ATCAGGCCTGAGACAGGCAATACACCATGATCACCTTTCCTTTCTTGTGCAACT
TTTTGTTTTCCTAGAACTGGAAAAAAAATGTGTTCAATTTTTTTTTTTTTTTTTTTTT
TTTTTTTTTTTTTTTTTTTTTTGAGACGGAGTTTTGCTCTGTCACCAGGCTGGAGT
GCAGTGGTGCGATCTCAGCTCACTGCAAGCTCCGCCTCCCGGGTTCAAGTGAT
TCTCCTGCCTCAGCCTCCTGAGTAGCTGGGACTACAGGTGCGTGCCACCACGC
CCAGCTAATTTTTGTATTTTTAGTAGAGACGGGGTTTCACCATGTTGGCCGGGA
TGGTCTCGATCTCTTGACCTCGTGATCCACCGACCTTGGCCTOCCAAAGTGCTG
GGATTACAGGCGTAAGCCACCATCCCAGCCCTGTGTTTGATTATATTTGGTCTG
GTTTTCTATCTACTTAACAGTGTGAACCCTAAATCATTCATCAATTGTATCTTTTG
TCAAGACGTCAATTCCATTAGATATTTTATCAATTTCTTTTTTTTTTTTTTTTTGAG
ACATAGTCTTGCTCTGTTGCCCAGGCTGGAGTGCCATGGCGCGATCTCGGCTC
ACTGCAAGCTCCACCTCCTGGGTTCTCCTGCCTCAGCCTCCCGAGTAGCTGGG
ACTACAGGTGCCCACCACCACATCCGACTAATTTTTTGTATTTTTTTTTTTTTTTT
TGAGATGGAGTCTCACTCTGTTGCCCAGGCTGGAGTGCAGTGGTGCGATCTCG
GCTCACTGAAAGCTCCATTTCTCCTGCCTCAGCCTOCCAAGTAGCTGGGACTCA
CACCATTCTCCTGCCTCGGCCTOCCAAGTAGCTGGGACTATAGGCACCCACCA
CCACGCCCGGCTAATTTTTTGTATTTTTAGTAGAGGCGGGGTTTCACCGTGTTA
GCCAGGATGGTCTCCATCTCCTGACCTCGCGATCCGCCGGCCTCGGCCTOCCA
AAGTGCTGGGATTACAGGCGTGAGCCACCACACCTGGCCTCTCATCAATTTCAT
TTCTTGAAGAAATTTCTTCAGGAGCCCACTGACCTGGTCCAGTTTGCACTAGTA
GCCCTCTGCCAGGGGACAGACCTGTCATGCTGGCATCTTCCTTCACCATCATCT
TGTAGAATCCTTTTGCCTCTGTCCTGGTTAGAAGTCCTGTTTCCTGTACTTGACT
CCTTTTCTTGACTTATTCCTTTGTTTTCATGGAGCAGGTCCTTCAGTAACTGCCT
TAGAAAGGTCATGAGAGGCCAGAGACAGTGGCTCACTCCTGTAATCCCAGCAC
TTTGGGAGGCCTTGAGTCCAGGAGTTTGAGACCAGCCTGGGCAATATACTGAT
ACCTTATCTCTACCAAAAATATAAAAATTAGCTGGCCATGGTGGTGCATGCTTGT
AGTACCAGCTACTCGGAAGGCTGAGGCAAGAGAATTGCTTGTACCCAGGAGGC
AGAGATTGCAGTCAGCCTAGATCATGCCACTGAACTCCAGCCTGGGCGACAGA
GTGAGACTCCATTTAAAAAAATAATTAATTAAATAATAATCTAAAAAAAAACTTTTT
TTTTTTTTTGAGATGAAGTGTCACTCTTGTCCCCCAGGCTGGAGTGCAATGGCG
TGATCTTGGCTCACTGCAACCTCCGCTTCCCGGGTTCAAGGATTCTCCTGCCTC
AGCATCCCAAGTAGCTGGGATTACAGGCACCTCCCATCACACCTGGCTAATTTT
TGTATTTTTAGTAGAGACGGGGTTTCACCATGTTGGCCAGGCTGGTCTCAAACT
CCTGACCTCAGGTGATCTGCCCGCCTTGGCCTOCCAAAGCGCTGGGATTACAG
GCATGAGCCACCGCGCCCAGCATCTAAAAAAAAAATTTAAAATAAGGTCATGGG
AAGTATATTTTTTGAGACTTTTGTGTGTCTCACCTTGAGTTGATAGCTTAGCTAG
GTATCCCATGCCAGGTTAGAAATCATTATTCTTCAGAATCTTGAAGGCACTTTTC
CATTGTCCTTCTTCCAGCATATATCGTTGCCATTTAGAAGGATGAAACCATCTGA
TTTCTGATCCTCTGATGTAATGTTTTTCTTTCTGAAAGTTTATAGAATTTTTACTTT
ATCCTCAGTGTGGTGTATTTTCATTCATGGCTCTAGGCACTTAGTATGTCTCTTC
ATTCTGAAAACATATGTCTTTCAATTCAGTGAAAAAGAATTATTTATTTAATTATTT
ATTTCTCTCCATTTTCTCTCTTGGGATTTTCTGTTATTGGATTTTGTTGCCCCCTG
AATTGGTCCCCTAATTTAATTCCCTTTTCTCTTATATTTTCCATCTCTTTACCTTTT
TCCTTTGGTTTCCAAGATATTCCCTCCACTTTATGTTCCAATCCTTCTATTGAGTT
TTTCATTTCTGTTCATGTAGTTTTAATTCCCACAAGCTCCTTACTTTAAATTTTTTT
TTTAATGAGACAGCATCTCACTATGTTGTCCAGGCTAGTTTCGAACTCCTGGGT
TCAAGCAATCCTCCTGCCTCGGCCTCCCAAAGTGCTGGGATTATAGGCGTAAG
CCACCCTGTCTGGCCTGTGCAAATGTTTTAAATGCATGTGTGTGTGACTGTGAG
TGTGAATATATGTGTATGTGTGTGTGTGAGTTTGTGTGTGAATGTGAGTATATAT
GTGTTTGACCGTGTGTTTGTGAGTGTGTTTGCATGTGTGACCGTGAGTGTATGT
ATAGTGTGAATGCATGTGTGTGTTTATGTGTGTCTGTGAATGTGAGTGTATGTGT
GTGTGTTTGAGTGTATGTGTGTGTGTGTGCAAGTGAATGCATGTGTGTATGACC
GTGAGTGTATGTGTGAGTGTTTAGGTTGTGTATGTGTGTGCAAGTGTTTGGATG
TGTGTGTGTGACCGTGAGTGTATGTGTGAGTGAGTGGGTTGCGTGTGCGTGGA
TGTGGGTCTGTGCCTCTCCCTTCCTGAATCCTCCCCCTCTGCACTTTCTCCACA
GTGGAACACACGGGCCAAGCGGGAGAAGCTGACAGAGCTGATGTTCGAGCAG
TACAACATTCCTGCCTTCTTCTTATGCAAGACGGCTGTGCTCACCGCGTATCCT
CTGGACTGAGCTGCTCTGACTGGGTGGAGGGCTGGGGTGGGGCCTGTGCTGA
GCTCAGCTGTGATGAGCTGGAGGGTCAGGCCTGGTTCTCTGAAGATCAGTGTC
CCAATGGGCAGACACCTCCCTCTGCCCCACTAGTGCAGAAGGCCCAGTGGACT
GAGCACCTCCGACTCTGGGGTGGCCAGATCAGCGGCCCCTTTCGGTTCCCCG
GATCAGGCTGAGCAGACAGTGAGCTGGGCCCGGGAGAGAGTAGGGGCTGGAA
CTGGACAGAGGAGGGAGACGTGTAGAACACTGCGGCAGTCCCTTCCCAAGAA
CTCCTTCAACACTGACTGTTTTTAGGATCTGCCTTGCATTGAACCCTGTGCTATG
CACTGGGGTGATAGCGACCCTAATTTCAGGGAATCTGTAGGACACAACCAAAAA
TAAAGGGGCTGGCCAGGCGCGGTGGCTCACGCCTGTAATCCCAGCATTTTGG
GAGGCCAAGGCGGGCAGATCACTTGAGGTCAGGAGTTAGAGACCAGTCCGGC
CAACATGGCAAAAGCCCGTGTCTACTAAAAATGGAAAAATTAGCCAGGCATAGT
GGTGGGCACCTGTAGTCCCAGCTACTTGGGAGGCTGAGGCAGGAGAATCTCTT
GAACCCAGGAGGCAGAGGTTGCAGTGAGCTATTGCATTCCAGCCTGGGCGACA
AAACGAAACTCAGTTTCAAAAAAAAAAAAATAACTAAAGAAGTAACTGGGGTCG
CCGGGTGTGGTGGCTCATGCCTGTAATCCCGGCACTTTGGGAGGCCGAGGCG
GGCAGATCACGAGGTCAGGAGATCGAGACCATCCTGGCTAACACGGTGAAATC
CCATCTCTACTAAAAATACGAAAAATTGGCCGGGCGTGGTGGCAGGCACCTGT
AGTCCCAGCTACTCGGGAGGCTGAGGCAGGAGAATGGCATGAACCCGGGAGG
CGGAGCTTGCAGTGAGCTGAGATTGCGCCACTGCACTCCAGCCTGGGAGACA
GCAAGACTCCATCTCAAAAAAAAAAAAAAAGAAGTAACTGGGGTCTCAGAGCCT
TTCTCCAGGAGATGAGTCATCACCAGGGGGAGAGATTTGCAGACTGGGGATGG
GCTGGACCCCGGTAGAGCAGAGAGTGACCAGACAAGATCTGGGCCCAGGCCC
CTCACTTGCTGCTGCTGTCTCTTTGACTTACCCATCCGATGCCAGCTTTGCAAA
CGGGCGGTCCACTGGCCTCGTGCTGGACAGTGGAGCCACCCACACCACGGCC
ATTCCAGTACATGACGGCTACGTTCTGCAGCAAGGTGGGGCTTCGGCAGGGG
GTGGGGGTGCTCCAGGAGGACCAGGTCCTGACAGTGCCTTCCTTCTAGGCATC
GTCAAGTCCCCTCTGGCAGGGGACTTCATCTCCATGCAGTGCCGGGAGCTGTT
CCAGGAGATGGCCATTGACATCATCCCACCTTACATGATCGCAGCCAAGGTGG
GGCCTCTGGGGACATGGAGCTGCCACTAGAGTCCTGGCCACTCGCCCCATGG
CCGTCAACCCTCTGACACATGAGATATCAGAGCAGAGAGCAGCAGGCTCCCCA
GTGGGTCCTGGCTCCAGTTCCTCCTTTTATAATCGAGGCATCCGAAATCCAGAC
AGGCTGAGGGATATGCTGAGGTCATGAAACCAGCTGGCAGACAGCAGGATGTG
TGAGGGCCAGTGTGGTGGGTTCTAGGGTCCAATATAGACCCAGGTTCAAGTTC
TGCCTCTGCCGTGCAAGAGCTGCAGGACCGGTCAGGCATGGTGGCTCACACC
TGTAATCCCAGCACTTCGGGAGGCTGAGGCAGGCAGATCGCTTTGAGCTCACT
TCGAGACCAGCCTGGGCAACATGGTGAAGCCACGTCTCTACTAAAACTGCAAG
AACTAGCCAAGCATGGTGGTGCATGCCTGTAGTCCCAGCTACTTGGGAGGCTG
AGACTGGAGAATCACCTGAACCCCGGAGGCAGAGGTTGCAGTGAGCCGAGAT
CGCGCCACTGCACTCCAGCCTGGGCGACCGAGTGAGACTCCATCTGAAAAAAA
AAAAAAAAAAGAGCTGTGTGACCAACAGCATGATATCAAAGCTCCCTGGGCCTC
AGGTTTGTCTTATGTAAAGTGGAACGGCAGTAGGCAGTAGCCAGGGTGCTGTG
AGTATGAAGTGACACTGTGTGCTTAGCTCCATTCCGTGCCCTCACATGGATCAT
GACCCCCTAGGAGGCCATTCCCTTGAGGTTCTGTCACTGCTGTATTCCTGGTGT
TCTCCAGATCAGCTGCACCCACCGCATGAAAGGCACACATGGAGAGAGAGGCA
TGCCCCATTCTCTCTCCTCTCCATCAGGGAAGGGTGAGAAACCCCTCTTGCAG
GCAGCACAGCAGAGCCTGCAGCGCCAGGTAGCTGCAGAGCTGGGAACAGTGC
CAGGAAGGTGGGGGTGGGGGTCAGGTGGGCTAGGAAAGGGTGTTCCTACACT
GTGCCCCCACAGGAGCCTGTCCGGGAGGGTGCCCCCCCAAACTGGAAGAAGA
AGGAGAAGCTACCCCAGGTCTCCAAGTCCTGGCATAACTACATGTGTAATGTGA
GCCTCTGGAGAGGGCCACCTGAAACCCCCCTCCCCTGCCATGAATCGGGGGC
GGGACAGAGGCCCCGCATGGGAGGGGGAGCAGGGAGGGGGAGCGGGGCAG
GGGGTGCTGCTCACGGGGAAGGCAGGCCCTCACCACCTCCGCTGGGATTTCA
GGAGGTGATCCAGGACTTCCAGGCCTCCGTGCTGCAGGTCTCAGACTCCCCCT
ACGATGAACAGTGAGTGTGGCTCAGGGCTTTGGGGTGGGAGAGAGGGCAGAA
CCGGCACTCTTGGGGTGGGCTGGGGGGCTCTGGTGCTGCCCACGCACTGATC
CTTCTTGAGCACCCCAGGCAGAGTCCTGCTCCCTTCTGGGTAGGGTGGCTGCA
CAAATGCCCACAGTGCACTACGAGATGCCCAATGGCTACAATACAGACTACGG
CGCCGAGCGACTCCGCATCCCTGAGGGCCTGTTTGATCCCTCGAACGTCAAGG
TTTGGCTGTGTGGCGAGGACTGGGGTGGGTGCTGGCTGCAGTCCCTGGATCC
TGGGGCGTGGGGGAGGCTGGGAAGGATTGCAGGGGTCTGGGGGGAAGGGGG
AGAGAGCCCACAGCCCCAGTCACCTCTCTCTGCAGGGCCTGTCGGGGAACAC
CATGTTGGGTGTGGGCCACGTGGTGACCACCAGCATCGGCATGTGTGACATTG
ATATTCGCCCGGTGAGGCCAGGCCGGAGCTGAGGCTCGGCAAAGGGGCCCAG
GGGAAGTGGGGAGACGGGGTTGGGGGGCTGCCCCCGCAGCTCACTCCTTCTC
CCTGCTCTCAGGGCCTGTACGGGAGTGTCATTGTCACCGGCGGGAACACACTG
CTGCAGGGCTTCACTGACAGGCTCAATCGAGAGCTTTCCCAGAAGACCCCACC
GGTAGGAGCCCACCCCTGACCCAGAGCCCTGGACACCCGTCCTGGCTCAGCT
GTCCAGGACTGAGTCCCTTCATCCTTCCCATGTCCTTCCTTCTGTTCTGGCCAC
CAGAGCAGAACCCCTGCCCCTGTGGCTTCCCAAGTCTTCCCCTCTCCCCCAAG
CCCTGTCCCTTCCCAGAACCTAGGTGTCTGCTTCCCCCAGCCCCTTTTTACCCC
CAGAGCATGCGACTGAAACTCATTGCCAGCAACAGCACCATGGAGCGCAAGTT
CAGCCCCTGGATCGGGGGTTCCATCCTGGCCTCACTGGTGAGAGGAAAGGGA
CCTGGCCTAGCACTGACTGTGGGGGAGAGGGGAGACCCAGACAGTGCCCGCC
ACTTTGACTGCCTCTTCGTTCATTCATTCATTCAACAAACATTTAGTTTTGAGAAA
TGTCAGTCAATGAATGTTCCCAGAAGGGTGAACAGGACACAAATCGGGGGGCA
GGGGCCCGGTATGGTGGCCCACACCTGTCATCCCAGCACTTTGGGTGGCTGA
AGTGGGGGGATGGCTTGAGGCTAGGAGTTGAAAACCCAGCAGGGGAACATAG
CAAGACCCATCTCTACTTTTACAAACTGGGGAGAAGAGGCTGCGCACAGTGGC
TCATGCCGGTAATCCCTGCAGTTTGGGAGGCCGAGACGGGTGGATCACTTGAG
CTCAGGAGTTCGAGACCAGCCTGGCCAACATGACGAAACCTTGTCTCTACTAAA
AATCCAAAAATTAGCCAGGCATGGTGGTGCATGCCTGTAATCCCAGCTATTCAG
GAGGCTGAGGCAGGAGAATCTCTTGAACCCAGGAGACGGAGACTGCAGTGAG
CTGAGATCGTGCCACTGCACTCCAGCCTGGGTGACGCAGTAAGACTCTCAAAA
AAAAAAAAAAATTGGGAGTAGAGGGACAGGTAGTTGGAGAGATTGGTCAGGAT
GAGATTTTGGTGATACACCCACTGTGCTCAGTCAGCAGAGGGGAGACCCTGAT
GTTTTGGGGCAAGGGAGTGATGTGGCAATGCAGGCATGATTGGAAAGACTGCC
AGGCATGAGTCTTTGGGGTCGGGGTATGAGCTGGGGTCAAGGGGACCTTACAA
GGAGGCTGTTTTGATTGTCCCGGAGTCATAGCATGGGTAGCAGTGGGGATGGG
GTGGACAGCAAGGGGCAACTAGTAAAGGAGGCAGGCAGGTTGGTTCTGGGGC
AGGAGGTGGTAAGTTCAGTCTGGACCTGCTGCTGGGTTCGAAGGAAGGTACTG
AGCAGGGAGCTGGAGTTGCAACCCGCATCTGGAGGGTATCAGTCACACAGAG
GGGACAGTGGGGCCATGACAGACAAGGAGGACATCCACGAGAAAGGAAGTCC
CCCAGGGAGACAGAAACCAGGGTCAGAAAGACAGAGGCTGGCAGTCTGAACTT
AGCTCAGGGCTGAAGCATGTCAGCCACACAGTTCTTGTTTTTTTCCTTTTTTTGT
TTTTGTTTTTGTTTTCGAGACAGTCTCACTGTCGCCCAGGCTGGAGTGCAGTGG
AGCAATCTTGGCTCACTGCAACCTCCATCTCCTGGGTTCAAGCGATTCTTCTGC
CTCAGCCTCCCGAGTAGCTGGGATTACATGCGTGAGCCACCACGCCTGGCTAA
TTTTTGGATTGTTAATAGAGATGGGGTTTCACCGTGTTGGCTAAGCTTGTCTCAA
ACTCCTGACCTCAAGTGATCTGCCCATCTTGGCCTCCCAAAGTGCTGGGATTAC
AGGTGTGAGCCACCATGCCCGGCCTGAAATTAAGTATTAATAATATATCCTGGA
CAAGTGGAGTGGCTCACACCTGTAATCCCAGCACTTTGAGAGGCCAAGGGGGG
AGGATCATTTGAGCCCAAAATTTCTAGACCAGCCTGGGCAACAAAGCGAGACCT
CATCTCTACAAAAAATTTAAAAAGCAACTGGCAATGTGGCGCATACCTGTAGTC
CCACCTACTTGGGAGGCCAAGGTTGGAGGATCACTGGAACCTGGAAGGTTGAG
GCTGCAGTGAGCTGTGCCACTGCATTCCAACCTGGGTGACAGAGCAAGATCCT
GTCTCAAAAAAAACCCAAAAAAATTGGGTGTGTATTTTCCACTTAGAGCACGTCT
CAATTTGGACCAGGGCTGGTGAGCTACAGGAAGCTAGTGGCCACTGCATTGGA
CAGCACATGTGTGTAGGCTGAGAAAGATCAAAGAAGCCCAGCCGTGGTAGCTC
ACGCCTGTAATCCCAACATTATGGGAGGCTGAGGTGGAACGATTACTTGAGCC
CAGGAGTTTGAGACCAGGCTGGGCAACATAGTGAGACACCATCTCTACAAAAA
ATAAAAATTAGGCTGGGCGCGGTGGCTCACGCTTGTAATCCCAGCACTTTGGG
AGGCCGAGGCGGGCGGATCATCTGAGGTCAGGAGTTTGAGACCAGCTTGGCC
AACATGGCGAAACCCTGTCTCTACTAAAAATACAAAAATTAGCCGGGCGTGTTG
GCACGCGCCTGTAATCCCAGCTACCCAGCAGGCTGAAGCAGGAGAATCGCTG
GAACCTGGGAGGCGGAGGCTGCAGTGAGCCAAGATCGCACCACTGCATTCCA
GCCTGGGCGACAGAGCAAGACTCCATCTCAAAAAAATAAAATAAAAATAAAAAT
AATTAAAATTAAAATTTTAAAAAAATAGAGAGAAGGGACACTCTGAGATGCTCAT
TGCAGGAACCCTCAAGGGGCAAGTTGTAATGCCGAAGGCTGCAGATGGGGTG
CCACAGTGTTGGAATGGTGAGGTGGAGGGAATATCTTCCTTGGGAAAGAGGGA
GACTGGCGGTGTTCCAAAGCCCTGATGGAGTGAGAAGAGGACACCTGGTGGG
GAAGGACTGTGTGTGTGTAGGGGGTGTTGAGTGCCAGGTGCAGGTTGCCTGG
CCTGGGGCTGTGCGTGCCATCCCCCCAGCTCAGGAACAGGGCCTAGCTCAGG
GCAGAGACTGCTGTTCAATAATACTTGTTGAATGAATGAATTATTGATTGAATGA
TATGAAGAGGATACTCTCCCTCTGAATATGAAAGGGGACAGACCGAGGAGGGG
AGGATGAGGTGGGGAGGGGCTTCAGAGGGATGTGAACCCAAGGGGTGGTTGG
GGCCTGTTGGAGGATTCCCAGGGGCTGCACCTGCCTGTCCACACCTCCCTCCA
AGGCCACCCTGACCCTGATATTACCCCCTCCACCCAGGGCACTTTCCAGCAGA
TGTGGATCTCCAAGCAGGAATATGAGGAGGGGGGGAAGCAGTGCGTGGAGCG
AAAGTGCCCCTGATGGCACTCCTCCCCACACACCTGCTCCCAAGCTCAGATGG
AAGTCCCTTAACCCCCATGCCACATTGCCCCCCTCCTCCTTTCCCTCTTGTCCT
CATTAATGGTGATGTTTCTGGGTTGAAAGAAGTAAAAATGTTTTAAGAAAAAAAA
ACTL6B wild-type mature mRNA sequence
SEQ ID NO: 2
GAGTCCCGCCCCGCCAGGGATCCCGGGAGCTGTCCGGCCGCCTCGGTGCTGATCCCGCCACC

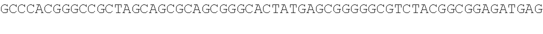

GTGGGGGCGCTGGTCTTTGACATTGGCTCCTTCTCAGTCCGCGCTGGGTACGCTGGGGAGGA
CTGTCCCAAGGCTGACTTCCCCACCACAGTGGGGCTGCTGGCCGCGGAGGAGGGGGGCGGGC
TGGAGCTGGAGGGGGACAAAGAGAAGAAAGGGAAGATCTTCCACATCGACACCAATGCCCTG
CACGTGCCTCGGGATGGAGCGGAGGTCATGTCGCCCCTCAAGAATGGCATGATCGAGGACTG
GGAGTGCTTCCGAGCCATCCTGGATCACACCTACAGCAAACACGTCAAGTCTGAGCCAAACC
TGCACCCAGTGCTCATGTCCGAGGCTCCGTGGAACACACGGGCCAAGCGGGAGAAGCTGACA
GAGCTGATGTTCGAGCAGTACAACATTCCTGCCTTCTTCTTATGCAAGACGGCTGTGCTCAC
CGCCTTTGCAAACGGGCGGTCCACTGGCCTCGTGCTGGACAGTGGAGCCACCCACACCACGG
CCATTCCAGTACATGACGGCTACGTTCTGCAGCAAGGCATCGTCAAGTCCCCTCTGGCAGGG
GACTTCATCTCCATGCAGTGCCGGGAGCTGTTCCAGGAGATGGCCATTGACATCATCCCACC
TTACATGATCGCAGCCAAGGAGCCTGTCCGGGAGGGTGCCCCCCCAAACTGGAAGAAGAAGG
AGAAGCTACCCCAGGTCTCCAAGTCCTGGCATAACTACATGTGTAATGAGGTGATCCAGGAC
TTCCAGGCCTCCGTGCTGCAGGTCTCAGACTCCCCCTACGATGAACAGGTGGCTGCACAAAT
GCCCACAGTGCACTACGAGATGCCCAATGGCTACAATACAGACTACGGCGCCGAGCGACTCC
GCATCCCTGAGGGCCTGTTTGATCCCTCGAACGTCAAGGGCCTGTCGGGGAACACCATGTTG
GGTGTGGGCCACGTGGTGACCACCAGCATCGGCATGTGTGACATTGATATTCGCCCGGGCCT
GTACGGGAGTGTCATTGTCACCGGCGGGAACACACTGCTGCAGGGCTTCACTGACAGGCTCA
ATCGAGAGCTTTCCCAGAAGACCCCACCGAGCATGCGACTGAAACTCATTGCCAGCAACAGC
ACCATGGAGCGCAAGTTCAGCCCCTGGATCGGGGGTTCCATCCTGGCCTCACTGGGCACTTT
CCAGCAGATGTGGATCTCCAAGCAGGAATATGAGGAGGGCGGGAAGCAGTGCGTGGAGCGAA

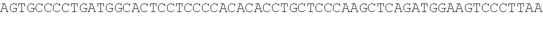

CCCCCATGCCACATTGCCCCCCTCCTCCTTTCCCTCTTGTCCTCATTAATGGTGATGTTTCT
GGGTTGAAAGAAGTAAAAATGTTTTAAGAAAAAAAA
ACTL6B wild-type protein sequence
SEQ ID NO: 3
MSGGVYGGDEVGALVFDIGSFSVRAGYAGEDCPKADFPTTVGLLAAEEGGGLELEGDKEKKG
KIFHIDTNALHVPRDGAEVMSPLKNGMIEDWECFRAILDHTYSKHVKSEPNLHPVLMSEAPW
NTRAKREKLTELMFEQYNIPAFFLCKTAVLTAFANGRSTGLVLDSGATHTTAIPVHDGYVLQ
QGIVKSPLAGDFISMQCRELFQEMAIDIIPPYMIAAKEPVREGAPPNWKKKEKLPQVSKSWH
NYMCNEVIQDFQASVLQVSDSPYDEQVAAQMPTVHYEMPNGYNTDYGAERLRIPEGLFDPSN
VKGLSGNTMLGVGHVVTTSIGMCDIDIRPGLYGSVIVTGGNTLLQGFTDRLNRELSQKTPPS
MRLKLIASNSTMERKFSPWIGGSILASLGTFQQMWISKQEYEEGGKQCVERKCP*
ACTL6B mutant mature mRNA
SEQ ID NO: 4
GAGTCCCGCCCCGCCAGGGATCCCGGGAGCTGTCCGGCCGCCTCGGTGCTGATCCCGCCACC

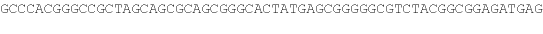

GTGGGGGCGCTGGTCTTTGACATTGGCTCCTTCTCAGTCCGCGCTGGGTACGCTGGGGAGGA
CTGTCCCAAGGCTGACTTCCCCACCACAGTGGGGCTGCTGGCCGCGGAGGAGGGGGGCGGGC
TGGAGCTGGAGGGGGACAAAGAGAAGAAAGGGAAGATCTTCCACATCGACACCAATGCCCTG
CACGTGCCTCGGGATGGAGCGGAGGTCATGTCGCCCCTCAAGAATGGCATGATCGAGGACTG
GGAGTGCTTCCGAGCCATCCTGGATCACACCTACAGCAAACACGTCAAGTCTGAGCCAAACC

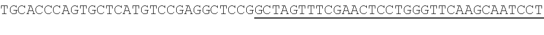

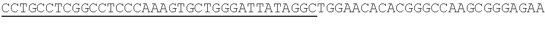

GCTGACAGAGCTGATGTTCGAGCAGTACAACATTCCTGCCTTCTTCTTATGCAAGACGGCTG
TGCTCACCGCCTTTGCAAACGGGCGGTCCACTGGCCTCGTGCTGGACAGTGGAGCCACCCAC
ACCACGGCCATTCCAGTACATGACGGCTACGTTCTGCAGCAAGGCATCGTCAAGTCCCCTCT
GGCAGGGGACTTCATCTCCATGCAGTGCCGGGAGCTGTTCCAGGAGATGGCCATTGACATCA
TCCCACCTTACATGATCGCAGCCAAGGAGCCTGTCCGGGAGGGTGCCCCCCCAAACTGGAAG
AAGAAGGAGAAGCTACCCCAGGTCTCCAAGTCCTGGCATAACTACATGTGTAATGAGGTGAT
CCAGGACTTCCAGGCCTCCGTGCTGCAGGTCTCAGACTCCCCCTACGATGAACAGGTGGCTG
CACAAATGCCCACAGTGCACTACGAGATGCCCAATGGCTACAATACAGACTACGGCGCCGAG
CGACTCCGCATCCCTGAGGGCCTGTTTGATCCCTCGAACGTCAAGGGCCTGTCGGGGAACAC
CATGTTGGGTGTGGGCCACGTGGTGACCACCAGCATCGGCATGTGTGACATTGATATTCGCC
CGGGCCTGTACGGGAGTGTCATTGTCACCGGCGGGAACACACTGCTGCAGGGCTTCACTGAC
AGGCTCAATCGAGAGCTTTCCCAGAAGACCCCACCGAGCATGCGACTGAAACTCATTGCCAG
CAACAGCACCATGGAGCGCAAGTTCAGCCCCTGGATCGGGGGTTCCATCCTGGCCTCACTGG
GCACTTTCCAGCAGATGTGGATCTCCAAGCAGGAATATGAGGAGGGCGGGAAGCAGTGCGTG

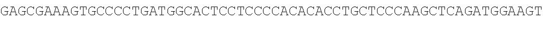

CCCTTAACCCCCATGCCACATTGCCCCCCTCCTCCTTTCCCTCTTGTCCTCATTAATGGTGA
TGTTTCTGGGTTGAAAGAAGTAAAAATGTTTTAAGAAAAAAAA
ACTL6B mutant protein sequence
SEQ ID NO: 5
MSGGVYGGDEVGALVFDIGSFSVRAGYAGEDCPKADFPTTVGLLAAEEGGGLELEGDKEKKG
KIFHIDTNALHVPRDGAEVMSPLKNGMIEDWECFRAILDHTYSKHVKSEPNLHPVLMSEAPA
SFELLGSSNPPASASQSAGIIGWNTRAKREKLTELMFEQYNIPAFFLCKTAVLTAFANGRST
GLVLDSGATHTTAIPVHDGYVLQQGIVKSPLAGDFISMQCRELFQEMAIDIIPPYMIAAKEP
VREGAPPNWKKKEKLPQVSKSWHNYMCNEVIQDFQASVLQVSDSPYDEQVAAQMPTVHYEMP
NGYNTDYGAERLRIPEGLFDPSNVKGLSGNTMLGVGHVVTTSIGMCDIDIRPGLYGSVIVTG
GNTLLQGFTDRLNRELSQKTPPSMRLKLIASNSTMERKFSPWIGGSILASLGTFQQMWISKQ
EYEEGGKQCVERKCP*
ASO target sequences
SEQ ID NO: 6-97
ASO sequences
SEQ ID NO: 98-189
ASO target sequences
SEQ ID NO: 190-193
ASO sequences
SEQ ID NO: 194-197
SEQ ID NO: 198
TCCACACTGAACAAACC
Target region 1
SEQ ID NO: 199
GAGACAGCATCTCACTATGTTGTCCAGGCTAGTTTCGAACTCCTGGGTTCAAGCA
ATCCTCCTGCCTCGGCCTCCCAAAGTGCTGGGATTATAGGCGTAAGCCACCCTGT
CTGGCCTGTGCAAATGTTTTAAATGCATGTGTGTGTGACTGTGAGTGTGAATATA
TGTGTATGTGTGTGTGTGAGTTTGTGTGTGAATGTGAGTATAT
Target region 2
SEQ ID NO: 200
ACTCCTGGGTTCAAGCAATCCTCCTGCCTCGGCCTCCCAAAGTGCTGGGATTATA
GGCGTAAGCCACCCTGTCTGGCCTGTGCAAATGTTTTAAATGCATGTGTGTGTGA
CTGTGAGTGTGAATATATGTGTATGTGTGTGTGTGAGT
Target region 3
SEQ ID NO: 201
TCCTGGGTTCAAGCAATCCTCCTGCCTCGGCCTCCCAAAGTGCTGGGATTATAGG
CGTAAGCCACCCTGTCTGGCCTGTGCAAATGTTTTAAATGCATGTGTGTGTGACT
GTGAGT
Target region 4
SEQ ID NO: 202
TCCTGGGTTCAAGCAATCCTCCTGCCTCGGCCTCCCAAAGTGCTGGGATTATAGG
CGTAAGCCACCCTGTCTGGCC
Target region 5
SEQ ID NO: 203
GGGTTCAAGCAATCCTCCTGCCTCGGCCTCCCAAAGTGCTGGGATTATAGGCGTA
A
Target region 6
SEQ ID NO: 204
AAGCAATCCTCCTGCCTCGGCC
Target region 7
SEQ ID NO: 205
CTGGGATTATAGGCGTAA

1. An antisense oligonucleotide actin-like 6B (ACTL6B) splice modulator, wherein said antisense oligonucleotide splice modulator is 8 to 40 nucleotides in length and comprises a contiguous nucleotide sequence of at least 8 nucleotides in length which is complementary to the ACTL6B precursor-mRNA.

2. The antisense oligonucleotide splice modulator of claim 1, wherein the ACTL6B precursor-mRNA has the sequence of SEQ ID NO: 1.

3. The antisense oligonucleotide splice modulator of claim 1 or claim 2, wherein the antisense oligonucleotide splice modulator is capable of increasing the expression of actin-like 6B (ACTL6B) in a TDP-43 depleted cell.

4. The antisense oligonucleotide splice modulator of claim 3, wherein ACTL6B is encoded by the nucleotide sequence of SEQ ID NO: 2, or a fragment or variant thereof.

5. The antisense oligonucleotide splice modulator of claim 3 or claim 4, wherein the ACTL6B protein has the sequence of SEQ ID NO: 3, or a fragment or variant thereof.

6. The antisense oligonucleotide splice modulator of any one of claims 1-5, wherein the antisense oligonucleotide splice modulator is capable of decreasing expression of a ACTL6B mutant polypeptide in a TDP-43 depleted cell.

7. The antisense oligonucleotide splice modulator of claim 6, wherein the ACTL6B mutant polypeptide is a splicing variant of ACTL6B.

8. The antisense oligonucleotide splice modulator of claim 7, wherein the ACTL6B mutant polypeptide comprises a polypeptide sequence encoded by an additional exon, when compared to the wild-type ACTL6B polypeptide sequence.

9. The antisense oligonucleotide splice modulator of claim 8, wherein the ACTL6B mutant polypeptide comprises an insertion, when compared to the wild-type ACTL6B polypeptide sequence.

10. The antisense oligonucleotide splice modulator of claim 9, wherein the insertion is an insertion of about 23 amino acids.

11. The antisense oligonucleotide splice modulator of any one of claims 6-10, wherein the ACTL6B mutant polypeptide is encoded by the nucleotide sequence of SEQ ID NO: 4, or a fragment or variant thereof.

12. The antisense oligonucleotide splice modulator of any one of claims 6-11, wherein the ACTL6B mutant polypeptide has the sequence of SEQ ID NO: 5, or a fragment or variant thereof.

13. The antisense oligonucleotide splice modulator of any one of claims 6-12, wherein the contiguous nucleotide sequence is complementary to a splice enhancer site in the ACTL6B precursor-mRNA.

14. The antisense oligonucleotide splice modulator of any one of claims 1 to 13, wherein the contiguous nucleotide sequence is complementary to SEQ ID NO 199.

15. The antisense oligonucleotide splice modulator of any one of claims 1 to 14, wherein the contiguous nucleotide sequence is complementary to SEQ ID NO 200.

16. The antisense oligonucleotide splice modulator of any one of claims 1 to 15, wherein the contiguous nucleotide sequence is complementary to SEQ ID NO 201.

17. The antisense oligonucleotide splice modulator of any one of claims 1 to 16, wherein the contiguous nucleotide sequence is complementary to SEQ ID NO 202.

18. The antisense oligonucleotide splice modulator of any one of claims 1 to 17, wherein the contiguous nucleotide sequence is complementary to SEQ ID NO 203.

19. The antisense oligonucleotide splice modulator of any one of claims 1 to 18, wherein the contiguous nucleotide sequence is complementary to SEQ ID NO 204.

20. The antisense oligonucleotide splice modulator of any one of claims 1 to 19, wherein the contiguous nucleotide sequence is complementary to SEQ ID NO 205.

21. The antisense oligonucleotide splice modulator of one of claims 1 to 20, wherein the contiguous nucleotide sequence is complementary to a sequence selected from SEQ ID NOs 6-97 and 190-193.

22. The antisense oligonucleotide splice modulator of claim 21, wherein the contiguous nucleotide sequence is complementary to a sequence selected from SEQ ID NOs:26, 28, 29, 30, 31, 32, 33, 38, 39, 46, 47, 48, 52, 53, 55 and 72.

23. The antisense oligonucleotide splice modulator of claim 22, wherein the contiguous nucleotide sequence is complementary to a sequence selected from SEQ ID NOs: 28, 29, 30, 31, 32, 33 and 47.

24. The antisense oligonucleotide splice modulator of any one of claims 1 to 23, wherein the contiguous nucleotide sequence is a sequence selected from SEQ ID Nos 98-189 and 194-197, or at least 10 contiguous nucleotides thereof.

25. The antisense oligonucleotide splice modulator of claim 24, wherein the contiguous nucleotide sequence is a sequence selected from SEQ ID NOs 118, 120, 121, 122, 123, 124, 125, 130, 131, 138, 139, 140, 144, 145, 147 and 164, or at least 10 contiguous nucleotides thereof.

26. The antisense oligonucleotide splice modulator of claim 25, wherein the contiguous nucleotide sequence is a sequence selected from SEQ ID NOs 120, 121, 122, 123, 124, 125 and 139, or at least 10 contiguous nucleotides thereof.

27. The antisense oligonucleotide splice modulator of any one of claims 1 to 26, wherein the contiguous nucleotide sequence is at least 12 nucleotides in length.

28. The antisense oligonucleotide splice modulator of any one of claims 1 to 27, wherein the contiguous nucleotide sequence is at least 13, 14, 15, 16, 17, 18, 19, 20, 21, 22, 23, 24, 25, 26, 27, 28, 29, 30, 31 or 32 nucleotides in length.

29. The antisense oligonucleotide splice modulator of any one of claims 1 to 28, wherein the contiguous nucleotide sequence is the same length as the antisense oligonucleotide splice modulator.

30. The antisense oligonucleotide splice modulator of any one of claims 1 to 29, wherein the antisense oligonucleotide splice modulator is isolated, purified or manufactured.

31. The antisense oligonucleotide splice modulator of any one of claims 1 to 30, wherein the contiguous nucleotide sequence comprises one or more modified nucleosides.

32. The antisense oligonucleotide splice modulator of any one of claims 1 to 31, wherein the antisense oligonucleotide splice modulator is a morpholino antisense oligonucleotide.

33. The antisense splice modulator oligonucleotide of claim 31 or claim 32, wherein the one or more modified nucleosides, is a 2′ sugar modified nucleoside, such as a 2′ sugar modified nucleoside independently selected from the group consisting of 2′-O-alkyl-RNA; 2′-O-methyl RNA (2′-OMe); 2′-alkoxy-RNA; 2′-O-methoxyethyl-RNA (2′-MOE); 2′-amino-DNA; 2′-fluro-RNA; 2′-fluoro-DNA; arabino nucleic acid (ANA); 2′-fluoro-ANA; locked nucleic acid (LNA), or any combination thereof.

34. The antisense oligonucleotide splice modulator of claim 33, wherein the 2′ sugar modified nucleoside is an affinity enhancing 2′ sugar modified nucleoside.

35. The antisense oligonucleotide splice modulator of any one of claims 1 to 34, wherein the contiguous nucleotide sequence of the antisense oligonucleotide splice modulator comprises 2′-O-methoxyethyl-RNA (2′-MOE) nucleosides.

36. The antisense oligonucleotide splice modulator of claim 35, wherein all the nucleosides of the contiguous nucleotide sequence are 2′-O-methoxyethyl-RNA (2′-MOE) nucleosides, optionally linked by phosphorothioate internucleoside linkages.

37. The antisense oligonucleotide splice modulator of any one of claims 31 to 36, wherein one or more of the modified nucleosides is a locked nucleic acid nucleoside (LNA), such as an LNA nucleoside selected from the group consisting of constrained ethyl nucleoside (cEt), and β-D-oxy-LNA.

38. The antisense oligonucleotide splice modulator of claim 37, wherein the contiguous nucleotide sequence of the antisense oligonucleotide splice modulator comprises or consists of LNA nucleosides and DNA nucleosides.

39. The antisense oligonucleotide splice modulator of any one of claims 1 to 38, wherein the contiguous nucleotide sequence is at least 75% complementary to the ACTL6B precursor-mRNA sequence.

40. The antisense oligonucleotide splice modulator of claim 39, wherein the contiguous nucleotide sequence is at least 80%, at least 85%, at least 90% or at least 95% complementary to the ACTL6B precursor-mRNA sequence.

41. The antisense oligonucleotide splice modulator of any one of claims 1 to 38 wherein the contiguous nucleotide sequence is fully complementary to the ACTL6B precursor-mRNA.

42. The antisense oligonucleotide splice modulator of any one of claims 1 to 40, wherein the contiguous nucleotide sequence comprises 1, 2, 3 or more mismatches to the ACTL6B precursor-mRNA sequence.

43. The antisense oligonucleotide splice modulator of any one of claims 1 to 42, wherein the Gibbs free energy of the antisense oligonucleotide splice modulator to a complementary target RNA is lower than about −10ΔG, such as lower than about −15 ΔG, such as lower than about −17 ΔG.

44. The antisense oligonucleotide splice modulator of any one of claims 1 to 43, wherein the antisense oligonucleotide splice modulator does not comprise a region of more than 3, or more than 4, contiguous DNA nucleosides.

45. The antisense oligonucleotide splice modulator of any one of claims 1 to 44, wherein the antisense oligonucleotide splice modulator is not capable of mediating RNAseH cleavage.

46. The antisense oligonucleotide splice modulator of any one of claims 1 to 45, wherein the antisense oligonucleotide splice modulator or contiguous nucleotide sequence thereof is a mixmer or a totalmer.

47. The antisense oligonucleotide splice modulator of any one of claims 1 to 46, wherein the cytosine bases present in the antisense oligonucleotide splice modulator or contiguous nucleotide sequence thereof are independently selected from the group consisting of cytosine and 5-methyl cytosine.

48. The antisense oligonucleotide splice modulator of any one of claims 1 to 47, wherein the cytosine bases present in the antisense oligonucleotide splice modulator or contiguous nucleotide sequence thereof are 5-methyl cytosine.

49. The antisense oligonucleotide splice modulator of any one of claims 1 to 48, wherein one or more of the internucleoside linkages positioned between the nucleosides on the contiguous nucleotide sequence are modified.

50. The antisense oligonucleotide splice modulator of any one of claims 1 to 49, wherein at least about 75%, at least about 80%, at least about 85%, at least about 90%, at least about 95%, or about 100% of the internucleoside linkages positioned between the nucleosides on the contiguous nucleotide sequence are modified.

51. The antisense oligonucleotide splice modulator of any one of claims 1 to 50, wherein one or more, or all of the modified internucleoside linkages comprise a phosphorothioate linkage.

52. The antisense oligonucleotide splice modulator of any one of claims 1 to 51, wherein all the internucleoside linkages present in the antisense oligonucleotide splice modulator are phosphorothioate internucleoside linkages.

53. The antisense oligonucleotide splice modulator of any one of claims 1 to 52, wherein the antisense oligonucleotide splice modulator is covalently attached to at least one conjugate moiety.

54. The antisense oligonucleotide splice modulator of any one of claims 1 to 53, wherein the antisense oligonucleotide splice modulator is in the form of a pharmaceutically acceptable salt.

55. The antisense oligonucleotide splice modulator of claim 54, wherein the pharmaceutically acceptable salt is a sodium salt or a potassium salt.

56. A pharmaceutical composition comprising the antisense oligonucleotide splice modulator of any one of claims 1 to 55, and a pharmaceutically acceptable diluent, solvent, carrier, salt and/or adjuvant.

57. A method, such as an in vivo or in vitro method, for increasing ACTL6B expression in a cell, said method comprising administering an antisense oligonucleotide splice modulator of any one of claims 1 to 55, or the pharmaceutical composition of claim 56, in an effective amount to said cell.

58. The method of claim 57, wherein said cell expresses aberrant or exhibits depleted levels of TDP-43.

59. A method for treating or preventing a disease in a subject comprising administering a therapeutically or prophylactically effective amount of an antisense oligonucleotide splice modulator of any one of claims 1 to 55, or the pharmaceutical composition of claim 56 to a subject suffering from or susceptible to the disease.

60. The antisense oligonucleotide splice modulator of any one of claims 1 to 55, or the pharmaceutical composition of claim 56, for use as a medicament.

61. The antisense oligonucleotide splice modulator of any one of claims 1 to 55, or the pharmaceutical composition of claim 56, for use in the treatment or prevention of disease in a subject.

62. The antisense oligonucleotide splice modulator of any one of claims 1 to 55 or the pharmaceutical composition of claim 56, for the preparation of a medicament for treatment or prevention of a disease in a subject.

63. The method of any one of claims 57 to 59 or the antisense oligonucleotide splice modulator for use of any one of claims 60 to 62, wherein the disease is a neurological disorder selected from the group consisting of amyotrophic lateral sclerosis (ALS), frontotemporal lobar degeneration (FTLD), Progressive supranuclear palsy (PSP), Primary lateral sclerosis, Progressive muscular atrophy, Alzheimer's disease, Parkinsons disease, Autism, Hippocampal sclerosis dementia, Down syndrome, Huntington's disease, polyglutamine diseases, such as spinocerebellar ataxia 3, myopathies and Chronic Traumatic Encephalopathy.

64. The method or antisense oligonucleotide splice modulator for use of claim 63, wherein the disease is a neurological disorder selected from the group consisting of amyotrophic lateral sclerosis (ALS) and frontotemporal lobar degeneration (FTLD).


SEQUENCE LISTING (211 sequences)

SEQ ID NO: 1 - DNA, 13352 bp, synthetic construct
  misc_feature 1..13352: note = ACTL6B
  source 1..13352: mol_type = other DNA
gagtcccgccccgccagggatcccgggagctgtccggccgcctcggtgctgatcccgccaccgcccacgggccgctagcagcgcagcgggcactatgagcgggggcgtctacggcggaggtgagcgagcctcgggcccccggctcccgcagccctgcgttccaaacccgcgccatccatccgagctctgggcaccccggggcctgtcacctccgcgcacgagcgggtcgagtctcaggctcgggtctcccgccacccctgcgctcggtcctccccagacgccccgcgcgcacagggccctaccttgcaacccacctccacctggccccgggccccttcctttcccgtcgggagggtgccctgagcaaggccattgggcaagaagggaccctggcggtgtgatgcccccacccccagtcccaggacctcggactccaggcccagctcccaccggccccagactggtgttgaaagtggcgagactgtggctctgccctgactttctctcggagctacctagcgggagagagttcgggggaggtccccttacaggcctgtctccccgcacagatgaggtgggggcgctggtctttgacattggctccttctcagtccgcgctgggtacgctggggaggactgtcccaaggtgagccttccagtctcctctccaaatcctaggggcttcggacccttcttcaggctttccagtgacccagggctctcccagatcgcccagaagctctgctgagcgggaatatgggaataaacccaggggcggtctgaggaccctgctgtggggctggacaggggcaagagggtgacctctctccctgccaatactcccccaacccttccccaccaggctgacttccccaccacagtggggctgctggccgcggaggaggggggcgggctggagctggagggggacaaagagaagaaagggaagatcttccacatcgacaccaatgccctgcacgtgcctcgggatggagcggaggtcatgtcgcccctcaagaatggcatgagtaaggggcccccacccatcacctcctgacagaaaaggagcccgctccccacaatctgggtcatagcagcccccactccccgcggagtcttccttgcagccccagggttcttctggctgccagggttcttctcgctgggttcccttcccaggggcccaccaggtttctgggaaggggggcgttcccctggagctggactcactgaccctcctttgggcttggcctttctccctccttttcttcctgtccctgtcccctgcctgccttcttgcgtctctgcacgctgctcttctggccccagtcgaggactgggagtgcttccgagccatcctggatcacacctacagcaaacacgtcaagtctgagccaaacctgcacccagtgctcatgtccgaggctccggtgagtgcctcctccatcctcatgtccaaccctgatctccactgcacctccttcccaccatccacctctcctcctctcccctcaagccactgctctgagccacttcccttcccttttcagactcccctcacccatcccaaacaatttccttccttcttggctaagtctccatccctcctgctctcttcttgctctatgcactctgctgcccttctctgtctccaagtcctttttaaattttttttttttgacacagtctcactccgtcacccaggctggagtgcaatggtgagatctcagcctctgcctcccgacatcaaaggaatctcctgcctcagcctcctgagtatctgggattacaggcacccgccaccacgcctggctaattttttgtatttttagtagacagggggtttcgccttgttggccaggctggtctcgaactcctgacctcaggtgatccacccgccttggcctcccaaagtgttgggattacaggcatgaggcaccacgcccagcctccaagtccttttttgaccttttcacttattattattattattattattattattattattattatggtcaactttggtgagctggggatgttggctcacacctgtaatcccaacactttgggaagccaaggcaggaggatcacttgagcccaggagtttgagaccaccctaggcaatatggcaagacccgtctcttttattaaaacaataaaaattaaaaattttttgccgggtgtggtggctcacgcctgtaatcccagcactttgggaggccgaggcgggtggatcacgaggtcaagagatcaagaccatcctggataacacggtgaaaccccatctctactaaaaatacaaaaaattagccaggcatggtggcgggcacctgtagtcccagctactcgggaggctgaggcaggagaatggcgtgaacccgggaggcggagcttgcagtgagctgagatcgcaccactgcactccagcctgggcaacagagcgagactctgtctcaaaaaaaaaaaaaatttaaaacctcatttaaaaaaatgttcttacccacctccttataaattcctctttataaaagcaactgtttttctcctttttagctaatgttgggggtattttcttccatatctctaatactacacttaaattgctgcctgtggagtgtttagttttaggtattatcgctgaattttccactttagaaaatgatcgcttagctctcttttcctccccagcctatgccacacgtatttcttatcaccctcctttaagtacagtgggttttgttgttgttgttgttgttgtttggggttttgtttgttgtttgtttttgagactttgcctcaaaaacagggggtctgctcagtttgtcacccaggttggattgcagtggcacaattcttcagccttggtctcctgggctcaagtgatcctctcatttcagtctccagagtagctggaactacaggcataagccaccatgcccagctgatttttaaattttttttagagacagggtttcatcatgttgtccaggctggtcttgaattcctgggctcaagcaatctgcccgcctcggcctcccaaaaggctgggattacaggcgagatccaccacacccagcccagttgtattttagactttaaaagaaatcagtattcagtatttacattctttttaaattagaaataattttaagagaatcttttgcagagatggggtctcatcatattgcccaggctggcttcaaactcctgtgcttaagcgatccttctacttcagctaacaaagtgctgggattacaggcatgagtcactgtgctcagcccaatatttacactcctattactatgtatgctaatcaggcctgagacaggcaatacaccatgatcacctttcctttcttgtgcaactttttgttttcctagaactggaaaaaaaatgtgttcaatttttttttttttttttttttttttttttttttttttttttttgagacggagttttgctctgtcaccaggctggagtgcagtggtgcgatctcagctcactgcaagctccgcctcccgggttcaagtgattctcctgcctcagcctcctgagtagctgggactacaggtgcgtgccaccacgcccagctaatttttgtatttttagtagagacggggtttcaccatgttggccgggatggtctcgatctcttgacctcgtgatccaccgaccttggcctcccaaagtgctgggattacaggcgtaagccaccatcccagccctgtgtttgattatatttggtctggttttctatctacttaacagtgtgaaccctaaatcattcatcaattgtatcttttgtcaagacgtcaattccattagatattttatcaatttctttttttttttttttttgagacatagtcttgctctgttgcccaggctggagtgccatggcgcgatctcggctcactgcaagctccacctcctgggttctcctgcctcagcctcccgagtagctgggactacaggtgcccaccaccacatccgactaattttttgtatttttttttttttttttgagatggagtctcactctgttgcccaggctggagtgcagtggtgcgatctcggctcactgaaagctccatttctcctgcctcagcctcccaagtagctgggactcacaccattctcctgcctcggcctcccaagtagctgggactataggcacccaccaccacgcccggctaattttttgtatttttagtagaggcggggtttcaccgtgttagccaggatggtctccatctcctgacctcgcgatccgccggcctcggcctcccaaagtgctgggattacaggcgtgagccaccacacctggcctctcatcaatttcatttcttgaagaaatttcttcaggagcccactgacctggtccagtttgcactagtagccctctgccaggggacagacctgtcatgctggcatcttccttcaccatcatcttgtagaatccttttgcctctgtcctggttagaagtcctgtttcctgtacttgactccttttcttgacttattcctttgttttcatggagcaggtccttcagtaactgccttagaaaggtcatgagaggccagagacagtggctcactcctgtaatcccagcactttgggaggccttgagtccaggagtttgagaccagcctgggcaatatactgataccttatctctaccaaaaatataaaaattagctggccatggtggtgcatgcttgtagtaccagctactcggaaggctgaggcaagagaattgcttgtacccaggaggcagagattgcagtcagcctagatcatgccactgaactccagcctgggcgacagagtgagactccatttaaaaaaataattaattaaataataatctaaaaaaaaacttttttttttttttgagatgaagtgtcactcttgtcccccaggctggagtgcaatggcgtgatcttggctcactgcaacctccgcttcccgggttcaaggattctcctgcctcagcatcccaagtagctgggattacaggcacctcccatcacacctggctaatttttgtatttttagtagagacggggtttcaccatgttggccaggctggtctcaaactcctgacctcaggtgatctgcccgccttggcctcccaaagcgctgggattacaggcatgagccaccgcgcccagcatctaaaaaaaaaatttaaaataaggtcatgggaagtatattttttgagacttttgtgtgtctcaccttgagttgatagcttagctaggtatcccatgccaggttagaaatcattattcttcagaatcttgaaggcacttttccattgtccttcttccagcatatatcgttgccatttagaaggatgaaaccatctgatttctgatcctctgatgtaatgtttttctttctgaaagtttatagaatttttactttatcctcagtgtggtgtattttcattcatggctctaggcacttagtatgtctcttcattctgaaaacatatgtctttcaattcagtgaaaaagaattatttatttaattatttatttctctccattttctctcttgggattttctgttattggattttgttgccccctgaattggtcccctaatttaattcccttttctcttatattttccatctctttacctttttcctttggtttccaagatattccctccactttatgttccaatccttctattgagtttttcatttctgttcatgtagttttaattcccacaagctccttactttaaattttttttttaatgagacagcatctcactatgttgtccaggctagtttcgaactcctgggttcaagcaatcctcctgcctcggcctcccaaagtgctgggattataggcgtaagccaccctgtctggcctgtgcaaatgttttaaatgcatgtgtgtgtgactgtgagtgtgaatatatgtgtatgtgtgtgtgtgagtttgtgtgtgaatgtgagtatatatgtgtttgaccgtgtgtttgtgagtgtgtttgcatgtgtgaccgtgagtgtatgtatagtgtgaatgcatgtgtgtgtttatgtgtgtctgtgaatgtgagtgtatgtgtgtgtgtttgagtgtatgtgtgtgtgtgtgcaagtgaatgcatgtgtgtatgaccgtgagtgtatgtgtgagtgtttaggttgtgtatgtgtgtgcaagtgtttggatgtgtgtgtgtgaccgtgagtgtatgtgtgagtgagtgggttgcgtgtgcgtggatgtgggtctgtgcctctcccttcctgaatcctccccctctgcactttctccacagtggaacacacgggccaagcgggagaagctgacagagctgatgttcgagcagtacaacattcctgccttcttcttatgcaagacggctgtgctcaccgcgtatcctctggactgagctgctctgactgggtggagggctggggtggggcctgtgctgagctcagctgtgatgagctggagggtcaggcctggttctctgaagatcagtgtcccaatgggcagacacctccctctgccccactagtgcagaaggcccagtggactgagcacctccgactctggggtggccagatcagcggcccctttcggttccccggatcaggctgagcagacagtgagctgggcccgggagagagtaggggctggaactggacagaggagggagacgtgtagaacactgcggcagtcccttcccaagaactccttcaacactgactgtttttaggatctgccttgcattgaaccctgtgctatgcactggggtgatagcgaccctaatttcagggaatctgtaggacacaaccaaaaataaaggggctggccaggcgcggtggctcacgcctgtaatcccagcattttgggaggccaaggcgggcagatcacttgaggtcaggagttagagaccagtccggccaacatggcaaaagcccgtgtctactaaaaatggaaaaattagccaggcatagtggtgggcacctgtagtcccagctacttgggaggctgaggcaggagaatctcttgaacccaggaggcagaggttgcagtgagctattgcattccagcctgggcgacaaaacgaaactcagtttcaaaaaaaaaaaaataactaaagaagtaactggggtcgccgggtgtggtggctcatgcctgtaatcccggcactttgggaggccgaggcgggcagatcacgaggtcaggagatcgagaccatcctggctaacacggtgaaatcccatctctactaaaaatacgaaaaattggccgggcgtggtggcaggcacctgtagtcccagctactcgggaggctgaggcaggagaatggcatgaacccgggaggcggagcttgcagtgagctgagattgcgccactgcactccagcctgggagacagcaagactccatctcaaaaaaaaaaaaaaagaagtaactggggtctcagagcctttctccaggagatgagtcatcaccagggggagagatttgcagactggggatgggctggaccccggtagagcagagagtgaccagacaagatctgggcccaggcccctcacttgctgctgctgtctctttgacttacccatccgatgccagctttgcaaacgggcggtccactggcctcgtgctggacagtggagccacccacaccacggccattccagtacatgacggctacgttctgcagcaaggtggggcttcggcagggggtgggggtgctccaggaggaccaggtcctgacagtgccttccttctaggcatcgtcaagtcccctctggcaggggacttcatctccatgcagtgccgggagctgttccaggagatggccattgacatcatcccaccttacatgatcgcagccaaggtggggcctctggggacatggagctgccactagagtcctggccactcgccccatggccgtcaaccctctgacacatgagatatcagagcagagagcagcaggctccccagtgggtcctggctccagttcctccttttataatcgaggcatccgaaatccagacaggctgagggatatgctgaggtcatgaaaccagctggcagacagcaggatgtgtgagggccagtgtggtgggttctagggtccaatatagacccaggttcaagttctgcctctgccgtgcaagagctgcaggaccggtcaggcatggtggctcacacctgtaatcccagcacttcgggaggctgaggcaggcagatcgctttgagctcacttcgagaccagcctgggcaacatggtgaagccacgtctctactaaaactgcaagaactagccaagcatggtggtgcatgcctgtagtcccagctacttgggaggctgagactggagaatcacctgaaccccggaggcagaggttgcagtgagccgagatcgcgccactgcactccagcctgggcgaccgagtgagactccatctgaaaaaaaaaaaaaaaaagagctgtgtgaccaacagcatgatatcaaagctccctgggcctcaggtttgtcttatgtaaagtggaacggcagtaggcagtagccagggtgctgtgagtatgaagtgacactgtgtgcttagctccattccgtgccctcacatggatcatgaccccctaggaggccattcccttgaggttctgtcactgctgtattcctggtgttctccagatcagctgcacccaccgcatgaaaggcacacatggagagagaggcatgccccattctctctcctctccatcagggaagggtgagaaacccctcttgcaggcagcacagcagagcctgcagcgccaggtagctgcagagctgggaacagtgccaggaaggtgggggtgggggtcaggtgggctaggaaagggtgttcctacactgtgcccccacaggagcctgtccgggagggtgcccccccaaactggaagaagaaggagaagctaccccaggtctccaagtcctggcataactacatgtgtaatgtgagcctctggagagggccacctgaaacccccctcccctgccatgaatcgggggcgggacagaggccccgcatgggagggggagcagggagggggagcggggcagggggtgctgctcacggggaaggcaggccctcaccacctccgctgggatttcaggaggtgatccaggacttccaggcctccgtgctgcaggtctcagactccccctacgatgaacagtgagtgtggctcagggctttggggtgggagagagggcagaaccggcactcttggggtgggctggggggctctggtgctgcccacgcactgatccttcttgagcaccccaggcagagtcctgctcccttctgggtagggtggctgcacaaatgcccacagtgcactacgagatgcccaatggctacaatacagactacggcgccgagcgactccgcatccctgagggcctgtttgatccctcgaacgtcaaggtttggctgtgtggcgaggactggggtgggtgctggctgcagtccctggatcctggggcgtgggggaggctgggaaggattgcaggggtctggggggaagggggagagagcccacagccccagtcacctctctctgcagggcctgtcggggaacaccatgttgggtgtgggccacgtggtgaccaccagcatcggcatgtgtgacattgatattcgcccggtgaggccaggccggagctgaggctcggcaaaggggcccaggggaagtggggagacggggttggggggctgcccccgcagctcactccttctccctgctctcagggcctgtacgggagtgtcattgtcaccggcgggaacacactgctgcagggcttcactgacaggctcaatcgagagctttcccagaagaccccaccggtaggagcccacccctgacccagagccctggacacccgtcctggctcagctgtccaggactgagtcccttcatccttcccatgtccttccttctgttctggccaccagagcagaacccctgcccctgtggcttcccaagtcttcccctctcccccaagccctgtcccttcccagaacctaggtgtctgcttcccccagcccctttttacccccagagcatgcgactgaaactcattgccagcaacagcaccatggagcgcaagttcagcccctggatcgggggttccatcctggcctcactggtgagaggaaagggacctggcctagcactgactgtgggggagaggggagacccagacagtgcccgccactttgactgcctcttcgttcattcattcattcaacaaacatttagttttgagaaatgtcagtcaatgaatgttcccagaagggtgaacaggacacaaatcggggggcaggggcccggtatggtggcccacacctgtcatcccagcactttgggtggctgaagtggggggatggcttgaggctaggagttgaaaacccagcaggggaacatagcaagacccatctctacttttacaaactggggagaagaggctgcgcacagtggctcatgccggtaatccctgcagtttgggaggccgagacgggtggatcacttgagctcaggagttcgagaccagcctggccaacatgacgaaaccttgtctctactaaaaatccaaaaattagccaggcatggtggtgcatgcctgtaatcccagctattcaggaggctgaggcaggagaatctcttgaacccaggagacggagactgcagtgagctgagatcgtgccactgcactccagcctgggtgacgcagtaagactctcaaaaaaaaaaaaaaattgggagtagagggacaggtagttggagagattggtcaggatgagattttggtgatacacccactgtgctcagtcagcagaggggagaccctgatgttttggggcaagggagtgatgtggcaatgcaggcatgattggaaagactgccaggcatgagtctttggggtcggggtatgagctggggtcaaggggaccttacaaggaggctgttttgattgtcccggagtcatagcatgggtagcagtggggatggggtggacagcaaggggcaactagtaaaggaggcaggcaggttggttctggggcaggaggtggtaagttcagtctggacctgctgctgggttcgaaggaaggtactgagcagggagctggagttgcaacccgcatctggagggtatcagtcacacagaggggacagtggggccatgacagacaaggaggacatccacgagaaaggaagtcccccagggagacagaaaccagggtcagaaagacagaggctggcagtctgaacttagctcagggctgaagcatgtcagccacacagttcttgtttttttcctttttttgtttttgtttttgttttcgagacagtctcactgtcgcccaggctggagtgcagtggagcaatcttggctcactgcaacctccatctcctgggttcaagcgattcttctgcctcagcctcccgagtagctgggattacatgcgtgagccaccacgcctggctaatttttggattgttaatagagatggggtttcaccgtgttggctaagcttgtctcaaactcctgacctcaagtgatctgcccatcttggcctcccaaagtgctgggattacaggtgtgagccaccatgcccggcctgaaattaagtattaataatatatcctggacaagtggagtggctcacacctgtaatcccagcactttgagaggccaaggcgggaggatcatttgagcccaaaatttctagaccagcctgggcaacaaagcgagacctcatctctacaaaaaatttaaaaagcaactggcaatgtggcgcatacctgtagtcccacctacttgggaggccaaggttggaggatcactggaacctggaaggttgaggctgcagtgagctgtgccactgcattccaacctgggtgacagagcaagatcctgtctcaaaaaaaacccaaaaaaattgggtgtgtattttccacttagagcacgtctcaatttggaccagggctggtgagctacaggaagctagtggccactgcattggacagcacatgtgtgtaggctgagaaagatcaaagaagcccagccgtggtagctcacgcctgtaatcccaacattatgggaggctgaggtggaacgattacttgagcccaggagtttgagaccaggctgggcaacatagtgagacaccatctctacaaaaaataaaaattaggctgggcgcggtggctcacgcttgtaatcccagcactttgggaggccgaggcgggcggatcatctgaggtcaggagtttgagaccagcttggccaacatggcgaaaccctgtctctactaaaaatacaaaaattagccgggcgtgttggcacgcgcctgtaatcccagctacccagcaggctgaagcaggagaatcgctggaacctgggaggcggaggctgcagtgagccaagatcgcaccactgcattccagcctgggcgacagagcaagactccatctcaaaaaaataaaataaaaataaaaataattaaaattaaaattttaaaaaaatagagagaagggacactctgagatgctcattgcaggaaccctcaaggggcaagttgtaatgccgaaggctgcagatggggtgccacagtgttggaatggtgaggtggagggaatatcttccttgggaaagagggagactggcggtgttccaaagccctgatggagtgagaagaggacacctggtggggaaggactgtgtgtgtgtagggggtgttgagtgccaggtgcaggttgcctggcctggggctgtgcgtgccatccccccagctcaggaacagggcctagctcagggcagagactgctgttcaataatacttgttgaatgaatgaattattgattgaatgatatgaagaggatactctccctctgaatatgaaaggggacagaccgaggaggggaggatgaggtggggaggggcttcagagggatgtgaacccaaggggtggttggggcctgttggaggattcccaggggctgcacctgcctgtccacacctccctccaaggccaccctgaccctgatattaccccctccacccagggcactttccagcagatgtggatctccaagcaggaatatgaggagggcgggaagcagtgcgtggagcgaaagtgcccctgatggcactcctccccacacacctgctcccaagctcagatggaagtcccttaacccccatgccacattgcccccctcctcctttccctcttgtcctcattaatggtgatgtttctgggttgaaagaagtaaaaatgttttaagaaaaaaaa

SEQ ID NO: 2 - DNA, 1524 bp, synthetic construct
  misc_feature 1..1524: note = ACTL6B
  source 1..1524: mol_type = other DNA
gagtcccgccccgccagggatcccgggagctgtccggccgcctcggtgctgatcccgccaccgcccacgggccgctagcagcgcagcgggcactatgagcgggggcgtctacggcggagatgaggtgggggcgctggtctttgacattggctccttctcagtccgcgctgggtacgctggggaggactgtcccaaggctgacttccccaccacagtggggctgctggccgcggaggaggggggcgggctggagctggagggggacaaagagaagaaagggaagatcttccacatcgacaccaatgccctgcacgtgcctcgggatggagcggaggtcatgtcgcccctcaagaatggcatgatcgaggactgggagtgcttccgagccatcctggatcacacctacagcaaacacgtcaagtctgagccaaacctgcacccagtgctcatgtccgaggctccgtggaacacacgggccaagcgggagaagctgacagagctgatgttcgagcagtacaacattcctgccttcttcttatgcaagacggctgtgctcaccgcctttgcaaacgggcggtccactggcctcgtgctggacagtggagccacccacaccacggccattccagtacatgacggctacgttctgcagcaaggcatcgtcaagtcccctctggcaggggacttcatctccatgcagtgccgggagctgttccaggagatggccattgacatcatcccaccttacatgatcgcagccaaggagcctgtccgggagggtgcccccccaaactggaagaagaaggagaagctaccccaggtctccaagtcctggcataactacatgtgtaatgaggtgatccaggacttccaggcctccgtgctgcaggtctcagactccccctacgatgaacaggtggctgcacaaatgcccacagtgcactacgagatgcccaatggctacaatacagactacggcgccgagcgactccgcatccctgagggcctgtttgatccctcgaacgtcaagggcctgtcggggaacaccatgttgggtgtgggccacgtggtgaccaccagcatcggcatgtgtgacattgatattcgcccgggcctgtacgggagtgtcattgtcaccggcgggaacacactgctgcagggcttcactgacaggctcaatcgagagctttcccagaagaccccaccgagcatgcgactgaaactcattgccagcaacagcaccatggagcgcaagttcagcccctggatcgggggttccatcctggcctcactgggcactttccagcagatgtggatctccaagcaggaatatgaggagggcgggaagcagtgcgtggagcgaaagtgcccctgatggcactcctccccacacacctgctcccaagctcagatggaagtcccttaacccccatgccacattgcccccctcctcctttccctcttgtcctcattaatggtgatgtttctgggttgaaagaagtaaaaatgttttaagaaaaaaaa

SEQ ID NO: 3 - protein, 426 aa, synthetic construct
  REGION 1..426: note = ACTL6B
MSGGVYGGDEVGALVFDIGSFSVRAGYAGEDCPKADFPTTVGLLAAEEGGGLELEGDKEKKGKIFHIDTNALHVPRDGAEVMSPLKNGMIEDWECFRAILDHTYSKHVKSEPNLHPVLMSEAPWNTRAKREKLTELMFEQYNIPAFFLCKTAVLTAFANGRSTGLVLDSGATHTTAIPVHDGYVLQQGIVKSPLAGDFISMQCRELFQEMAIDIIPPYMIAAKEPVREGAPPNWKKKEKLPQVSKSWHNYMCNEVIQDFQASVLQVSDSPYDEQVAAQMPTVHYEMPNGYNTDYGAERLRIPEGLFDPSNVKGLSGNTMLGVGHVVTTSIGMCDIDIRPGLYGSVIVTGGNTLLQGFTDRLNRELSQKTPPSMRLKLIASNSTMERKFSPWIGGSILASLGTFQQMWISKQEYEEGGKQCVERKCP

SEQ ID NO: 4 - DNA, 1593 bp, synthetic construct
  misc_feature 1..1593: note = ACTL6B mutant
  source 1..1593: mol_type = other DNA
gagtcccgccccgccagggatcccgggagctgtccggccgcctcggtgctgatcccgccaccgcccacgggccgctagcagcgcagcgggcactatgagcgggggcgtctacggcggagatgaggtgggggcgctggtctttgacattggctccttctcagtccgcgctgggtacgctggggaggactgtcccaaggctgacttccccaccacagtggggctgctggccgcggaggaggggggcgggctggagctggagggggacaaagagaagaaagggaagatcttccacatcgacaccaatgccctgcacgtgcctcgggatggagcggaggtcatgtcgcccctcaagaatggcatgatcgaggactgggagtgcttccgagccatcctggatcacacctacagcaaacacgtcaagtctgagccaaacctgcacccagtgctcatgtccgaggctccggctagtttcgaactcctgggttcaagcaatcctcctgcctcggcctcccaaagtgctgggattataggctggaacacacgggccaagcgggagaagctgacagagctgatgttcgagcagtacaacattcctgccttcttcttatgcaagacggctgtgctcaccgcctttgcaaacgggcggtccactggcctcgtgctggacagtggagccacccacaccacggccattccagtacatgacggctacgttctgcagcaaggcatcgtcaagtcccctctggcaggggacttcatctccatgcagtgccgggagctgttccaggagatggccattgacatcatcccaccttacatgatcgcagccaaggagcctgtccgggagggtgcccccccaaactggaagaagaaggagaagctaccccaggtctccaagtcctggcataactacatgtgtaatgaggtgatccaggacttccaggcctccgtgctgcaggtctcagactccccctacgatgaacaggtggctgcacaaatgcccacagtgcactacgagatgcccaatggctacaatacagactacggcgccgagcgactccgcatccctgagggcctgtttgatccctcgaacgtcaagggcctgtcggggaacaccatgttgggtgtgggccacgtggtgaccaccagcatcggcatgtgtgacattgatattcgcccgggcctgtacgggagtgtcattgtcaccggcgggaacacactgctgcagggcttcactgacaggctcaatcgagagctttcccagaagaccccaccgagcatgcgactgaaactcattgccagcaacagcaccatggagcgcaagttcagcccctggatcgggggttccatcctggcctcactgggcactttccagcagatgtggatctccaagcaggaatatgaggagggcgggaagcagtgcgtggagcgaaagtgcccctgatggcactcctccccacacacctgctcccaagctcagatggaagtcccttaacccccatgccacattgcccccctcctcctttccctcttgtcctcattaatggtgatgtttctgggttgaaagaagtaaaaatgttttaagaaaaaaaa

SEQ ID NO: 5 - protein, 449 aa, synthetic construct
  REGION 1..449: note = ACTL6B mutant
MSGGVYGGDEVGALVFDIGSFSVRAGYAGEDCPKADFPTTVGLLAAEEGGGLELEGDKEKKGKIFHIDTNALHVPRDGAEVMSPLKNGMIEDWECFRAILDHTYSKHVKSEPNLHPVLMSEAPASFELLGSSNPPASASQSAGIIGWNTRAKREKLTELMFEQYNIPAFFLCKTAVLTAFANGRSTGLVLDSGATHTTAIPVHDGYVLQQGIVKSPLAGDFISMQCRELFQEMAIDIIPPYMIAAKEPVREGAPPNWKKKEKLPQVSKSWHNYMCNEVIQDFQASVLQVSDSPYDEQVAAQMPTVHYEMPNGYNTDYGAERLRIPEGLFDPSNVKGLSGNTMLGVGHVVTTSIGMCDIDIRPGLYGSVIVTGGNTLLQGFTDRLNRELSQKTPPSMRLKLIASNSTMERKFSPWIGGSILASLGTFQQMWISKQEYEEGGKQCVERKCP

SEQ ID NO: 6 - DNA, 18 bp, synthetic construct
  misc_feature 1..18: note = polynucleotide
  source 1..18: mol_type = other DNA
gagacagcatctcactat

SEQ ID NO: 7 - DNA, 18 bp, synthetic construct
  misc_feature 1..18: note = Synthetic polynucleotide
  source 1..18: mol_type = other DNA
gacagcatctcactatgt

SEQ ID NO: 8 - DNA, 18 bp, synthetic construct
  misc_feature 1..18: note = Synthetic polynucleotide
  source 1..18: mol_type = other DNA
cagcatctcactatgttg

SEQ ID NO: 9 - DNA, 18 bp, synthetic construct
  misc_feature 1..18: note = Synthetic polynucleotide
  source 1..18: mol_type = other DNA
gcatctcactatgttgtc

SEQ ID NO: 10 - DNA, 18 bp, synthetic construct
  misc_feature 1..18: note = Synthetic polynucleotide
  source 1..18: mol_type = other DNA
atctcactatgttgtcca

SEQ ID NO: 11 - DNA, 18 bp, synthetic construct
  misc_feature 1..18: note = Synthetic polynucleotide
  source 1..18: mol_type = other DNA
ctcactatgttgtccagg

SEQ ID NO: 12 - DNA, 18 bp, synthetic construct
  misc_feature 1..18: note = Synthetic polynucleotide
  source 1..18: mol_type = other DNA
cactatgttgtccaggct

SEQ ID NO: 13 - DNA, 18 bp, synthetic construct
  misc_feature 1..18: note = Synthetic polynucleotide
  source 1..18: mol_type = other DNA
ctatgttgtccaggctag

SEQ ID NO: 14 - DNA, 18 bp, synthetic construct
  misc_feature 1..18: note = Synthetic polynucleotide
  source 1..18: mol_type = other DNA
atgttgtccaggctagtt

SEQ ID NO: 15 - DNA, 18 bp, synthetic construct
  misc_feature 1..18: note = Synthetic polynucleotide
  source 1..18: mol_type = other DNA
gttgtccaggctagtttc

SEQ ID NO: 16 - DNA, 18 bp, synthetic construct
  misc_feature 1..18: note = Synthetic polynucleotide
  source 1..18: mol_type = other DNA
tgtccaggctagtttcga

SEQ ID NO: 17 - DNA, 18 bp, synthetic construct
  misc_feature 1..18: note = Synthetic polynucleotide
  source 1..18: mol_type = other DNA
tccaggctagtttcgaac

SEQ ID NO: 18 - DNA, 18 bp, synthetic construct
  misc_feature 1..18: note = Synthetic polynucleotide
  source 1..18: mol_type = other DNA
caggctagtttcgaactc

SEQ ID NO: 19 - DNA, 18 bp, synthetic construct
  misc_feature 1..18: note = Synthetic polynucleotide
  source 1..18: mol_type = other DNA
ggctagtttcgaactcct

SEQ ID NO: 20 - DNA, 18 bp, synthetic construct
  misc_feature 1..18: note = Synthetic polynucleotide
  source 1..18: mol_type = other DNA
ctagtttcgaactcctgg

SEQ ID NO: 21 - DNA, 18 bp, synthetic construct
  misc_feature 1..18: note = Synthetic polynucleotide
  source 1..18: mol_type = other DNA
agtttcgaactcctgggt

SEQ ID NO: 22 - DNA, 18 bp, synthetic construct
  misc_feature 1..18: note = Synthetic polynucleotide
  source 1..18: mol_type = other DNA
tttcgaactcctgggttc

SEQ ID NO: 23 - DNA, 18 bp, synthetic construct
  misc_feature 1..18: note = Synthetic polynucleotide
  source 1..18: mol_type = other DNA
tcgaactcctgggttcaa

SEQ ID NO: 24 - DNA, 18 bp, synthetic construct
  misc_feature 1..18: note = Synthetic polynucleotide
  source 1..18: mol_type = other DNA
gaactcctgggttcaagc

SEQ ID NO: 25 - DNA, 18 bp, synthetic construct
  misc_feature 1..18: note = Synthetic polynucleotide
  source 1..18: mol_type = other DNA
actcctgggttcaagcaa

SEQ ID NO: 26 - DNA, 18 bp, synthetic construct
  misc_feature 1..18: note = Synthetic polynucleotide
  source 1..18: mol_type = other DNA
tcctgggttcaagcaatc

SEQ ID NO: 27 - DNA, 18 bp, synthetic construct
  misc_feature 1..18: note = Synthetic polynucleotide
  source 1..18: mol_type = other DNA
ctgggttcaagcaatcct

SEQ ID NO: 28 - DNA, 18 bp, synthetic construct
  misc_feature 1..18: note = Synthetic polynucleotide
  source 1..18: mol_type = other DNA
gggttcaagcaatcctcc

SEQ ID NO: 29 - DNA, 18 bp, synthetic construct
  misc_feature 1..18: note = Synthetic polynucleotide
  source 1..18: mol_type = other DNA
gttcaagcaatcctcctg

SEQ ID NO: 30 - DNA, 18 bp, synthetic construct
  misc_feature 1..18: note = Synthetic polynucleotide
  source 1..18: mol_type = other DNA
tcaagcaatcctcctgcc

SEQ ID NO: 31 - DNA, 18 bp, synthetic construct
  misc_feature 1..18: note = Synthetic polynucleotide
  source 1..18: mol_type = other DNA
aagcaatcctcctgcctc

SEQ ID NO: 32 - DNA, 18 bp, synthetic construct
  misc_feature 1..18: note = Synthetic polynucleotide
  source 1..18: mol_type = other DNA
gcaatcctcctgcctcgg

SEQ ID NO: 33 - DNA, 18 bp, synthetic construct
  misc_feature 1..18: note = Synthetic polynucleotide
  source 1..18: mol_type = other DNA
aatcctcctgcctcggcc

SEQ ID NO: 34 - DNA, 18 bp, synthetic construct
  misc_feature 1..18: note = Synthetic polynucleotide
  source 1..18: mol_type = other DNA
tcctcctgcctcggcctc

SEQ ID NO: 35 - DNA, 18 bp, synthetic construct
  misc_feature 1..18: note = Synthetic polynucleotide
  source 1..18: mol_type = other DNA
ctcctgcctcggcctccc

SEQ ID NO: 36 - DNA, 18 bp, synthetic construct
  misc_feature 1..18: note = Synthetic polynucleotide
  source 1..18: mol_type = other DNA
cctgcctcggcctcccaa

SEQ ID NO: 37 - DNA, 18 bp, synthetic construct
  misc_feature 1..18: note = Synthetic polynucleotide
  source 1..18: mol_type = other DNA
tgcctcggcctcccaaag

SEQ ID NO: 38 - DNA, 18 bp, synthetic construct
  misc_feature 1..18: note = Synthetic polynucleotide
  source 1..18: mol_type = other DNA
cctcggcctcccaaagtg

SEQ ID NO: 39 - DNA, 18 bp, synthetic construct
  misc_feature 1..18: note = Synthetic polynucleotide
  source 1..18: mol_type = other DNA
tcggcctcccaaagtgct

SEQ ID NO: 40 - DNA, 18 bp, synthetic construct
  misc_feature 1..18: note = Synthetic polynucleotide
  source 1..18: mol_type = other DNA
ggcctcccaaagtgctgg

SEQ ID NO: 41 - DNA, 18 bp, synthetic construct
  misc_feature 1..18: note = Synthetic polynucleotide
  source 1..18: mol_type = other DNA
cctcccaaagtgctggga

SEQ ID NO: 42 - DNA, 18 bp, synthetic construct
  misc_feature 1..18: note = Synthetic polynucleotide
  source 1..18: mol_type = other DNA
tcccaaagtgctgggatt

SEQ ID NO: 43 - DNA, 18 bp, synthetic construct
  misc_feature 1..18: note = Synthetic polynucleotide
  source 1..18: mol_type = other DNA
ccaaagtgctgggattat

SEQ ID NO: 44 - DNA, 18 bp, synthetic construct
  misc_feature 1..18: note = Synthetic polynucleotide
  source 1..18: mol_type = other DNA
aaagtgctgggattatag

SEQ ID NO: 45 - DNA, 18 bp, synthetic construct
  misc_feature 1..18: note = Synthetic polynucleotide
  source 1..18: mol_type = other DNA
agtgctgggattataggc

SEQ ID NO: 46 - DNA, 18 bp, synthetic construct
  misc_feature 1..18: note = Synthetic polynucleotide
  source 1..18: mol_type = other DNA
tgctgggattataggcgt

SEQ ID NO: 47 - DNA, 18 bp, synthetic construct
  misc_feature 1..18: note = Synthetic polynucleotide
  source 1..18: mol_type = other DNA
ctgggattataggcgtaa

SEQ ID NO: 48 - DNA, 18 bp, synthetic construct
  misc_feature 1..18: note = Synthetic polynucleotide
  source 1..18: mol_type = other DNA
gggattataggcgtaagc

SEQ ID NO: 49 - DNA, 18 bp, synthetic construct
  misc_feature 1..18: note = Synthetic polynucleotide
  source 1..18: mol_type = other DNA
gattataggcgtaagcca

SEQ ID NO: 50 - DNA, 18 bp, synthetic construct
  misc_feature 1..18: note = Synthetic polynucleotide
  source 1..18: mol_type = other DNA
ttataggcgtaagccacc

SEQ ID NO: 51 - DNA, 18 bp, synthetic construct
  misc_feature 1..18: note = Synthetic polynucleotide
  source 1..18: mol_type = other DNA
ataggcgtaagccaccct

SEQ ID NO: 52 - DNA, 18 bp, synthetic construct
  misc_feature 1..18: note = Synthetic polynucleotide
  source 1..18: mol_type = other DNA
aggcgtaagccaccctgt

SEQ ID NO: 53 - DNA, 18 bp, synthetic construct
  misc_feature 1..18: note = Synthetic polynucleotide
  source 1..18: mol_type = other DNA
gcgtaagccaccctgtct

SEQ ID NO: 54 - DNA, 18 bp, synthetic construct
  misc_feature 1..18: note = Synthetic polynucleotide
  source 1..18: mol_type = other DNA
gtaagccaccctgtctgg

SEQ ID NO: 55 - DNA, 18 bp, synthetic construct
  misc_feature 1..18: note = Synthetic polynucleotide
  source 1..18: mol_type = other DNA
aagccaccctgtctggcc

SEQ ID NO: 56 - DNA, 18 bp, synthetic construct
  misc_feature 1..18: note = Synthetic polynucleotide
  source 1..18: mol_type = other DNA
gccaccctgtctggcctg

SEQ ID NO: 57 - DNA, 18 bp, synthetic construct
  misc_feature 1..18: note = Synthetic polynucleotide
  source 1..18: mol_type = other DNA
caccctgtctggcctgtg

SEQ ID NO: 58 - DNA, 18 bp, synthetic construct
  misc_feature 1..18: note = Synthetic polynucleotide
  source 1..18: mol_type = other DNA
ccctgtctggcctgtgca

SEQ ID NO: 59 - DNA, 18 bp, synthetic construct
  misc_feature 1..18: note = Synthetic polynucleotide
  source 1..18: mol_type = other DNA
ctgtctggcctgtgcaaa

SEQ ID NO: 60 - DNA, 18 bp, synthetic construct
  misc_feature 1..18: note = Synthetic polynucleotide
  source 1..18: mol_type = other DNA
gtctggcctgtgcaaatg

SEQ ID NO: 61 - DNA, 18 bp, synthetic construct
  misc_feature 1..18: note = Synthetic polynucleotide
  source 1..18: mol_type = other DNA
ctggcctgtgcaaatgtt

SEQ ID NO: 62 - DNA, 18 bp, synthetic construct
  misc_feature 1..18: note = Synthetic polynucleotide
  source 1..18: mol_type = other DNA
ggcctgtgcaaatgtttt

SEQ ID NO: 63 - DNA, 18 bp, synthetic construct
  misc_feature 1..18: note = Synthetic polynucleotide
  source 1..18: mol_type = other DNA
cctgtgcaaatgttttaa

SEQ ID NO: 64 - DNA, 18 bp, synthetic construct
  misc_feature 1..18: note = Synthetic polynucleotide
  source 1..18: mol_type = other DNA
tgtgcaaatgttttaaat

SEQ ID NO: 65 - DNA, 18 bp, synthetic construct
  misc_feature 1..18: note = Synthetic polynucleotide
  source 1..18: mol_type = other DNA
tgcaaatgttttaaatgc

SEQ ID NO: 66 - DNA, 18 bp, synthetic construct
  misc_feature 1..18: note = Synthetic polynucleotide
  source 1..18: mol_type = other DNA
caaatgttttaaatgcat

SEQ ID NO: 67 - DNA, 18 bp, synthetic construct
  misc_feature 1..18: note = Synthetic polynucleotide
  source 1..18: mol_type = other DNA
aatgttttaaatgcatgt

SEQ ID NO: 68 - DNA, 18 bp, synthetic construct
  misc_feature 1..18: note = Synthetic polynucleotide
  source 1..18: mol_type = other DNA
ttttaaatgcatgtgtgt

SEQ ID NO: 69 - DNA, 18 bp, synthetic construct
  misc_feature 1..18: note = Synthetic polynucleotide
  source 1..18: mol_type = other DNA
ttaaatgcatgtgtgtgt

SEQ ID NO: 70 - DNA, 18 bp, synthetic construct
  misc_feature 1..18: note = Synthetic polynucleotide
  source 1..18: mol_type = other DNA
aaatgcatgtgtgtgtga

SEQ ID NO: 71 - DNA, 18 bp, synthetic construct
  misc_feature 1..18: note = Synthetic polynucleotide
  source 1..18: mol_type = other DNA
atgcatgtgtgtgtgact

SEQ ID NO: 72 - DNA, 18 bp, synthetic construct
  misc_feature 1..18: note = Synthetic polynucleotide
  source 1..18: mol_type = other DNA
gcatgtgtgtgtgactgt

SEQ ID NO: 73 - DNA, 18 bp, synthetic construct
  misc_feature 1..18: note = Synthetic polynucleotide
  source 1..18: mol_type = other DNA
atgtgtgtgtgactgtga

SEQ ID NO: 74 - DNA, 18 bp, synthetic construct
  misc_feature 1..18: note = Synthetic polynucleotide
  source 1..18: mol_type = other DNA
gtgtgtgtgactgtgagt

SEQ ID NO: 75 - DNA, 18 bp, synthetic construct
  misc_feature 1..18: note = Synthetic polynucleotide
  source 1..18: mol_type = other DNA
gtgtgtgactgtgagtgt

SEQ ID NO: 76 - DNA, 18 bp, synthetic construct
  misc_feature 1..18: note = Synthetic polynucleotide
  source 1..18: mol_type = other DNA
gtgtgactgtgagtgtga

SEQ ID NO: 77 - DNA, 18 bp, synthetic construct
  misc_feature 1..18: note = Synthetic polynucleotide
  source 1..18: mol_type = other DNA
gtgactgtgagtgtgaat

SEQ ID NO: 78 - DNA, 18 bp, synthetic construct
  misc_feature 1..18: note = Synthetic polynucleotide
  source 1..18: mol_type = other DNA
gactgtgagtgtgaatat

SEQ ID NO: 79 - DNA, 18 bp, synthetic construct
  misc_feature 1..18: note = Synthetic polynucleotide
  source 1..18: mol_type = other DNA
ctgtgagtgtgaatatat

SEQ ID NO: 80 - DNA, 18 bp, synthetic construct
  misc_feature 1..18: note = Synthetic polynucleotide
  source 1..18: mol_type = other DNA
gtgagtgtgaatatatgt

SEQ ID NO: 81 - DNA, 18 bp, synthetic construct
  misc_feature 1..18: note = Synthetic polynucleotide
  source 1..18: mol_type = other DNA
gagtgtgaatatatgtgt

SEQ ID NO: 82 - DNA, 18 bp, synthetic construct
  misc_feature 1..18: note = Synthetic polynucleotide
  source 1..18: mol_type = other DNA
gtgaatatatgtgtatgt

SEQ ID NO: 83 - DNA, 18 bp, synthetic construct
  misc_feature 1..18: note = Synthetic polynucleotide
  source 1..18: mol_type = other DNA
gaatatatgtgtatgtgt

SEQ ID NO: 84 - DNA, 18 bp, synthetic construct
  misc_feature 1..18: note = Synthetic polynucleotide
  source 1..18: mol_type = other DNA
atatatgtgtatgtgtgt

SEQ ID NO: 85 - DNA, 18 bp, synthetic construct
  misc_feature 1..18: note = Synthetic polynucleotide
  source 1..18: mol_type = other DNA
atatgtgtatgtgtgtgt

SEQ ID NO: 86 - DNA, 18 bp, synthetic construct
  misc_feature 1..18: note = Synthetic polynucleotide
  source 1..18: mol_type = other DNA
atgtgtatgtgtgtgtgt

SEQ ID NO: 87 - DNA, 18 bp, synthetic construct
  misc_feature 1..18: note = Synthetic polynucleotide
  source 1..18: mol_type = other DNA
gtgtatgtgtgtgtgtga

SEQ ID NO: 88 - DNA, 18 bp, synthetic construct
  misc_feature 1..18: note = Synthetic polynucleotide
  source 1..18: mol_type = other DNA
gtatgtgtgtgtgtgagt

SEQ ID NO: 89 - DNA, 18 bp, synthetic construct
  misc_feature 1..18: note = Synthetic polynucleotide
  source 1..18: mol_type = other DNA
atgtgtgtgtgtgagttt

SEQ ID NO: 90 - DNA, 18 bp, synthetic construct
  misc_feature 1..18: note = Synthetic polynucleotide
  source 1..18: mol_type = other DNA
gtgtgtgtgagtttgtgt

SEQ ID NO: 91 - DNA, 18 bp, synthetic construct
  misc_feature 1..18: note = Synthetic polynucleotide
  source 1..18: mol_type = other DNA
gtgtgtgagtttgtgtgt

SEQ ID NO: 92 - DNA, 18 bp, synthetic construct
  misc_feature 1..18: note = Synthetic polynucleotide
  source 1..18: mol_type = other DNA
gtgtgagtttgtgtgtga

SEQ ID NO: 93 - DNA, 18 bp, synthetic construct
  misc_feature 1..18: note = Synthetic polynucleotide
  source 1..18: mol_type = other DNA
gtgagtttgtgtgtgaat

SEQ ID NO: 94 - DNA, 18 bp, synthetic construct
  misc_feature 1..18: note = Synthetic polynucleotide
  source 1..18: mol_type = other DNA
gtttgtgtgtgaatgtga

SEQ ID NO: 95 - DNA, 18 bp, synthetic construct
  misc_feature 1..18: note = Synthetic polynucleotide
  source 1..18: mol_type = other DNA
ttgtgtgtgaatgtgagt

SEQ ID NO: 96 - DNA, 18 bp, synthetic construct
  misc_feature 1..18: note = Synthetic polynucleotide
  source 1..18: mol_type = other DNA
gtgtgtgaatgtgagtat

SEQ ID NO: 97 - DNA, 18 bp, synthetic construct
  misc_feature 1..18: note = Synthetic polynucleotide
  source 1..18: mol_type = other DNA
gtgtgaatgtgagtatat

SEQ ID NO: 98 - DNA, 18 bp, synthetic construct
  misc_feature 1..18: note = Polynucleotide
  source 1..18: mol_type = other DNA
atagtgagatgctgtctc

SEQ ID NO: 99 - DNA, 18 bp, synthetic construct
  misc_feature 1..18: note = Polynucleotide
  source 1..18: mol_type = other DNA
acatagtgagatgctgtc

SEQ ID NO: 100 - DNA, 18 bp, synthetic construct
  misc_feature 1..18: note = Polynucleotide
  source 1..18: mol_type = other DNA
caacatagtgagatgctg

SEQ ID NO: 101 - DNA, 18 bp, synthetic construct
  misc_feature 1..18: note = Polynucleotide
  source 1..18: mol_type = other DNA
gacaacatagtgagatgc

SEQ ID NO: 102 - DNA, 18 bp, synthetic construct
  misc_feature 1..18: note = Polynucleotide
  source 1..18: mol_type = other DNA
tggacaacatagtgagat

SEQ ID NO: 103 - DNA, 18 bp, synthetic construct
  misc_feature 1..18: note = Polynucleotide
  source 1..18: mol_type = other DNA
cctggacaacatagtgag

SEQ ID NO: 104 - DNA, 18 bp, synthetic construct
  misc_feature 1..18: note = Polynucleotide
  source 1..18: mol_type = other DNA
agcctggacaacatagtg

SEQ ID NO: 105 - DNA, 18 bp, synthetic construct
  misc_feature 1..18: note = Polynucleotide
  source 1..18: mol_type = other DNA
ctagcctggacaacatag

SEQ ID NO: 106 - DNA, 18 bp, synthetic construct
  misc_feature 1..18: note = Polynucleotide
  source 1..18: mol_type = other DNA
aactagcctggacaacat

SEQ ID NO: 107 - DNA, 18 bp, synthetic construct
  misc_feature 1..18: note = Polynucleotide
  source 1..18: mol_type = other DNA
gaaactagcctggacaac

SEQ ID NO: 108 - DNA, 18 bp, synthetic construct
  misc_feature 1..18: note = Polynucleotide
  source 1..18: mol_type = other DNA
tcgaaactagcctggaca

SEQ ID NO: 109 - DNA, 18 bp, synthetic construct
  misc_feature 1..18: note = Polynucleotide
  source 1..18: mol_type = other DNA
gttcgaaactagcctgga

SEQ ID NO: 110 - DNA, 18 bp, synthetic construct
  misc_feature 1..18: note = Polynucleotide
  source 1..18: mol_type = other DNA
gagttcgaaactagcctg

SEQ ID NO: 111 - DNA, 18 bp, synthetic construct
  misc_feature 1..18: note = Polynucleotide
  source 1..18: mol_type = other DNA
aggagttcgaaactagcc

SEQ ID NO: 112 - DNA, 18 bp, synthetic construct
  misc_feature 1..18: note = Polynucleotide
  source 1..18: mol_type = other DNA
ccaggagttcgaaactag

SEQ ID NO: 113 - DNA, 18 bp, synthetic construct
  misc_feature 1..18: note = Polynucleotide
  source 1..18: mol_type = other DNA
acccaggagttcgaaact

SEQ ID NO: 114 - DNA, 18 bp, synthetic construct
  misc_feature 1..18: note = Polynucleotide
  source 1..18: mol_type = other DNA
gaacccaggagttcgaaa

SEQ ID NO: 115 - DNA, 18 bp, synthetic construct
  misc_feature 1..18: note = Polynucleotide
  source 1..18: mol_type = other DNA
ttgaacccaggagttcga

SEQ ID NO: 116 - DNA, 18 bp, synthetic construct
  misc_feature 1..18: note = Polynucleotide
  source 1..18: mol_type = other DNA
gcttgaacccaggagttc

SEQ ID NO: 117 - DNA, 18 bp, synthetic construct
  misc_feature 1..18: note = Polynucleotide
  source 1..18: mol_type = other DNA
ttgcttgaacccaggagt

SEQ ID NO: 118 - DNA, 18 bp, synthetic construct
  misc_feature 1..18: note = Polynucleotide
  source 1..18: mol_type = other DNA
gattgcttgaacccagga

SEQ ID NO: 119 - DNA, 18 bp, synthetic construct
  misc_feature 1..18: note = Polynucleotide
  source 1..18: mol_type = other DNA
aggattgcttgaacccag

SEQ ID NO: 120 - DNA, 18 bp, synthetic construct
  misc_feature 1..18: note = Polynucleotide
  source 1..18: mol_type = other DNA
ggaggattgcttgaaccc

SEQ ID NO: 121 - DNA, 18 bp, synthetic construct
  misc_feature 1..18: note = Polynucleotide
  source 1..18: mol_type = other DNA
caggaggattgcttgaac

SEQ ID NO: 122 - DNA, 18 bp, synthetic construct
  misc_feature 1..18: note = Polynucleotide
  source 1..18: mol_type = other DNA
ggcaggaggattgcttga

SEQ ID NO: 123 - DNA, 18 bp, synthetic construct
  misc_feature 1..18: note = Polynucleotide
  source 1..18: mol_type = other DNA
gaggcaggaggattgctt

SEQ ID NO: 124 - DNA, 18 bp, synthetic construct
  misc_feature 1..18: note = Polynucleotide
  source 1..18: mol_type = other DNA
ccgaggcaggaggattgc

SEQ ID NO: 125 - DNA, 18 bp, synthetic construct
  misc_feature 1..18: note = Polynucleotide
  source 1..18: mol_type = other DNA
ggccgaggcaggaggatt

SEQ ID NO: 126 - DNA, 18 bp, synthetic construct
  misc_feature 1..18: note = Polynucleotide
  source 1..18: mol_type = other DNA
gaggccgaggcaggagga

SEQ ID NO: 127 - DNA, 18 bp, synthetic construct
  misc_feature 1..18: note = Polynucleotide
  source 1..18: mol_type = other DNA
gggaggccgaggcaggag

SEQ ID NO: 128 - DNA, 18 bp, synthetic construct
  misc_feature 1..18: note = Polynucleotide
  source 1..18: mol_type = other DNA
ttgggaggccgaggcagg

SEQ ID NO: 129 - DNA, 18 bp, synthetic construct
  misc_feature 1..18: note = Polynucleotide
  source 1..18: mol_type = other DNA
ctttgggaggccgaggca

SEQ ID NO: 130 - DNA, 18 bp, synthetic construct
  misc_feature 1..18: note = Polynucleotide
  source 1..18: mol_type = other DNA
cactttgggaggccgagg

SEQ ID NO: 131 - DNA, 18 bp, synthetic construct
  misc_feature 1..18: note = Polynucleotide
  source 1..18: mol_type = other DNA
agcactttgggaggccga

SEQ ID NO: 132 - DNA, 18 bp, synthetic construct
  misc_feature 1..18: note = Polynucleotide
  source 1..18: mol_type = other DNA
ccagcactttgggaggcc

SEQ ID NO: 133 - DNA, 18 bp, synthetic construct
  misc_feature 1..18: note = Polynucleotide
  source 1..18: mol_type = other DNA
tcccagcactttgggagg

SEQ ID NO: 134 - DNA, 18 bp, synthetic construct
  misc_feature 1..18: note = Polynucleotide
  source 1..18: mol_type = other DNA
aatcccagcactttggga

SEQ ID NO: 135 - DNA, 18 bp, synthetic construct
  misc_feature 1..18: note = Polynucleotide
  source 1..18: mol_type = other DNA
ataatcccagcactttgg

SEQ ID NO: 136 - DNA, 18 bp, synthetic construct
  misc_feature 1..18: note = Polynucleotide
  source 1..18: mol_type = other DNA
ctataatcccagcacttt

SEQ ID NO: 137 - DNA, 18 bp, synthetic construct
  misc_feature 1..18: note = Polynucleotide
  source 1..18: mol_type = other DNA
gcctataatcccagcact

SEQ ID NO: 138 - DNA, 18 bp, synthetic construct
  misc_feature 1..18: note = Polynucleotide
  source 1..18: mol_type = other DNA
acgcctataatcccagca

SEQ ID NO: 139 - DNA, 18 bp, synthetic construct
  misc_feature 1..18: note = Polynucleotide
  source 1..18: mol_type = other DNA
ttacgcctataatcccag

SEQ ID NO: 140 - DNA, 18 bp, synthetic construct
  misc_feature 1..18: note = Polynucleotide
  source 1..18: mol_type = other DNA
gcttacgcctataatccc

SEQ ID NO: 141 - DNA, 18 bp, synthetic construct
  misc_feature 1..18: note = Polynucleotide
  source 1..18: mol_type = other DNA
tggcttacgcctataatc

SEQ ID NO: 142 - DNA, 18 bp, synthetic construct
  misc_feature 1..18: note = Polynucleotide
  source 1..18: mol_type = other DNA
ggtggcttacgcctataa

SEQ ID NO: 143 - DNA, 18 bp, synthetic construct
  misc_feature 1..18: note = Polynucleotide
  source 1..18: mol_type = other DNA
agggtggcttacgcctat

SEQ ID NO: 144 - DNA, 18 bp, synthetic construct
  misc_feature 1..18: note = Polynucleotide
  source 1..18: mol_type = other DNA
acagggtggcttacgcct

SEQ ID NO: 145 - DNA, 18 bp, synthetic construct
  misc_feature 1..18: note = Polynucleotide
  source 1..18: mol_type = other DNA
agacagggtggcttacgc

SEQ ID NO: 146 - DNA, 18 bp, synthetic construct
  misc_feature 1..18: note = Polynucleotide
  source 1..18: mol_type = other DNA
ccagacagggtggcttac

SEQ ID NO: 147 - DNA, 18 bp, synthetic construct
  misc_feature 1..18: note = Polynucleotide
  source 1..18: mol_type = other DNA
ggccagacagggtggctt

SEQ ID NO: 148 - DNA, 18 bp, synthetic construct
  misc_feature 1..18: note = Polynucleotide
  source 1..18: mol_type = other DNA
caggccagacagggtggc

SEQ ID NO: 149 - DNA, 18 bp, synthetic construct
  misc_feature 1..18: note = Polynucleotide
  source 1..18: mol_type = other DNA
cacaggccagacagggtg

SEQ ID NO: 150 - DNA, 18 bp, synthetic construct
  misc_feature 1..18: note = Polynucleotide
  source 1..18: mol_type = other DNA
tgcacaggccagacaggg

SEQ ID NO: 151 - DNA, 18 bp, synthetic construct
  misc_feature 1..18: note = Polynucleotide
  source 1..18: mol_type = other DNA
tttgcacaggccagacag

SEQ ID NO: 152 - DNA, 18 bp, synthetic construct
  misc_feature 1..18: note = Polynucleotide
  source 1..18: mol_type = other DNA
catttgcacaggccagac

SEQ ID NO: 153 - DNA, 18 bp, synthetic construct
  misc_feature 1..18: note = Polynucleotide
  source 1..18: mol_type = other DNA
aacatttgcacaggccag

SEQ ID NO: 154 - DNA, 18 bp, synthetic construct
  misc_feature 1..18: note = Polynucleotide
  source 1..18: mol_type = other DNA
aaaacatttgcacaggcc

SEQ ID NO: 155 - DNA, 18 bp, synthetic construct
  misc_feature 1..18: note = Polynucleotide
  source 1..18: mol_type = other DNA
ttaaaacatttgcacagg

SEQ ID NO: 156 - DNA, 18 bp, synthetic construct
  misc_feature 1..18: note = Polynucleotide
  source 1..18: mol_type = other DNA
atttaaaacatttgcaca

SEQ ID NO: 157 - DNA, 18 bp, synthetic construct
  misc_feature 1..18: note = Polynucleotide
  source 1..18: mol_type = other DNA
gcatttaaaacatttgca

SEQ ID NO: 158 - DNA, 18 bp, synthetic construct
  misc_feature 1..18: note = Polynucleotide
  source 1..18: mol_type = other DNA
atgcatttaaaacatttg

SEQ ID NO: 159 - DNA, 18 bp, synthetic construct
  misc_feature 1..18: note = Polynucleotide
  source 1..18: mol_type = other DNA
acatgcatttaaaacatt

SEQ ID NO: 160 - DNA, 18 bp, synthetic construct
  misc_feature 1..18: note = Polynucleotide
  source 1..18: mol_type = other DNA
acacacatgcatttaaaa

SEQ ID NO: 161 - DNA, 18 bp, synthetic construct
  misc_feature 1..18: note = Polynucleotide
  source 1..18: mol_type = other DNA
acacacacatgcatttaa

SEQ ID NO: 162 - DNA, 18 bp, synthetic construct
  misc_feature 1..18: note = Polynucleotide
  source 1..18: mol_type = other DNA
tcacacacacatgcattt

SEQ ID NO: 163 - DNA, 18 bp, synthetic construct
  misc_feature 1..18: note = Polynucleotide
  source 1..18: mol_type = other DNA
agtcacacacacatgcat

SEQ ID NO: 164 - DNA, 18 bp, synthetic construct
  misc_feature 1..18: note = Polynucleotide
  source 1..18: mol_type = other DNA
acagtcacacacacatgc

SEQ ID NO: 165 - DNA, 18 bp, synthetic construct
  misc_feature 1..18: note = Polynucleotide
  source 1..18: mol_type = other DNA
tcacagtcacacacacat

SEQ ID NO: 166 - DNA, 18 bp, synthetic construct
  misc_feature 1..18: note = Polynucleotide
  source 1..18: mol_type = other DNA
actcacagtcacacacac

SEQ ID NO: 167 - DNA, 18 bp, synthetic construct
  misc_feature 1..18: note = Polynucleotide
  source 1..18: mol_type = other DNA
acactcacagtcacacac

SEQ ID NO: 168 - DNA, 18 bp, synthetic construct
  misc_feature 1..18: note = Polynucleotide
  source 1..18: mol_type = other DNA
tcacactcacagtcacac

SEQ ID NO: 169 - DNA, 18 bp, synthetic construct
  misc_feature 1..18: note = Polynucleotide
  source 1..18: mol_type = other DNA
attcacactcacagtcac

SEQ ID NO: 170 - DNA, 18 bp, synthetic construct
  misc_feature 1..18: note = Polynucleotide
  source 1..18: mol_type = other DNA
atattcacactcacagtc

SEQ ID NO: 171 - DNA, 18 bp, synthetic construct
  misc_feature 1..18: note = Polynucleotide
  source 1..18: mol_type = other DNA
atatattcacactcacag

SEQ ID NO: 172 - DNA, 18 bp, synthetic construct
  misc_feature 1..18: note = Polynucleotide
  source 1..18: mol_type = other DNA
acatatattcacactcac

SEQ ID NO: 173 - DNA, 18 bp, synthetic construct
  misc_feature 1..18: note = Polynucleotide
  source 1..18: mol_type = other DNA
acacatatattcacactc

SEQ ID NO: 174 - DNA, 18 bp, synthetic construct
  misc_feature 1..18: note = Polynucleotide
  source 1..18: mol_type = other DNA
acatacacatatattcac

SEQ ID NO: 175 - DNA, 18 bp, synthetic construct
  misc_feature 1..18: note = Polynucleotide
  source 1..18: mol_type = other DNA
acacatacacatatattc

SEQ ID NO: 176 - DNA, 18 bp, synthetic construct
  misc_feature 1..18: note = Polynucleotide
  source 1..18: mol_type = other DNA
acacacatacacatatat

SEQ ID NO: 177 - DNA, 18 bp, synthetic construct
  misc_feature 1..18: note = Polynucleotide
  source 1..18: mol_type = other DNA
acacacacatacacatat

SEQ ID NO: 178 - DNA, 18 bp, synthetic construct
  misc_feature 1..18: note = Polynucleotide
  source 1..18: mol_type = other DNA
acacacacacatacacat

SEQ ID NO: 179 - DNA, 18 bp, synthetic construct
  misc_feature 1..18: note = Polynucleotide
  source 1..18: mol_type = other DNA
tcacacacacacatacac

SEQ ID NO: 180 - DNA, 18 bp, synthetic construct
  misc_feature 1..18: note = Polynucleotide
  source 1..18: mol_type = other DNA
actcacacacacacatac

SEQ ID NO: 181 - DNA, 18 bp, synthetic construct
  misc_feature 1..18: note = Polynucleotide
  source 1..18: mol_type = other DNA
aaactcacacacacacat

SEQ ID NO: 182 - DNA, 18 bp, synthetic construct
  misc_feature 1..18: note = Polynucleotide
  source 1..18: mol_type = other DNA
acacaaactcacacacac

SEQ ID NO: 183 - DNA, 18 bp, synthetic construct
  misc_feature 1..18: note = Polynucleotide
  source 1..18: mol_type = other DNA
acacacaaactcacacac

SEQ ID NO: 184 - DNA, 18 bp, synthetic construct
  misc_feature 1..18: note = Polynucleotide
  source 1..18: mol_type = other DNA
tcacacacaaactcacac

SEQ ID NO: 185 - DNA, 18 bp, synthetic construct
  misc_feature 1..18: note = Polynucleotide
  source 1..18: mol_type = other DNA
attcacacacaaactcac

SEQ ID NO: 186 - DNA, 18 bp, synthetic construct
  misc_feature 1..18: note = Polynucleotide
  source 1..18: mol_type = other DNA
tcacattcacacacaaac

SEQ ID NO: 187 - DNA, 18 bp, synthetic construct
  misc_feature 1..18: note = Polynucleotide
  source 1..18: mol_type = other DNA
actcacattcacacacaa

SEQ ID NO: 188 - DNA, 18 bp, synthetic construct
  misc_feature 1..18: note = Polynucleotide
  source 1..18: mol_type = other DNA
atactcacattcacacac

SEQ ID NO: 189 - DNA, 18 bp, synthetic construct
  misc_feature 1..18: note = Polynucleotide
  source 1..18: mol_type = other DNA
atatactcacattcacac

SEQ ID NO: 190 - DNA, 18 bp, synthetic construct
  misc_feature 1..18: note = Synthetic polynucleotide
  source 1..18: mol_type = other DNA
tgttttaaatgcatgtgt

SEQ ID NO: 191 - DNA, 18 bp, synthetic construct
  misc_feature 1..18: note = Synthetic polynucleotide
  source 1..18: mol_type = other DNA
gtgtgaatatatgtgtat

SEQ ID NO: 192 - DNA, 18 bp, synthetic construct
  misc_feature 1..18: note = Synthetic polynucleotide
  source 1..18: mol_type = other DNA
gtgtgtgtgtgagtttgt

SEQ ID NO: 193 - DNA, 18 bp, synthetic construct
  misc_feature 1..18: note = Synthetic polynucleotide
  source 1..18: mol_type = other DNA
gagtttgtgtgtgaatgt

SEQ ID NO: 194 - DNA, 18 bp, synthetic construct
  misc_feature 1..18: note = Polynucleotide
  source 1..18: mol_type = other DNA
acacatgcatttaaaaca

SEQ ID NO: 195 - DNA, 18 bp, synthetic construct
  misc_feature 1..18: note = Polynucleotide
  source 1..18: mol_type = other DNA
atacacatatattcacac

SEQ ID NO: 196 - DNA, 18 bp, synthetic construct
  misc_feature 1..18: note = Polynucleotide
  source 1..18: mol_type = other DNA
acaaactcacacacacac

SEQ ID NO: 197 - DNA, 18 bp, synthetic construct
  misc_feature 1..18: note = Polynucleotide
  source 1..18: mol_type = other DNA
acattcacacacaaactc

SEQ ID NO: 198 - DNA, 17 bp, synthetic construct
  misc_feature 1..17: note = Synthetic polynucleotide
  source 1..17: mol_type = other DNA
tccacactgaacaaacc

SEQ ID NO: 199 - DNA, 208 bp, synthetic construct
  misc_feature 1..208: note = Polynucleotide
  source 1..208: mol_type = other DNA
gagacagcatctcactatgttgtccaggctagtttcgaactcctgggttcaagcaatcctcctgcctcggcctcccaaagtgctgggattataggcgtaagccaccctgtctggcctgtgcaaatgttttaaatgcatgtgtgtgtgactgtgagtgtgaatatatgtgtatgtgtgtgtgtgagtttgtgtgtgaatgtgagtatat

SEQ ID NO: 200 - DNA, 148 bp, synthetic construct
  misc_feature 1..148: note = Polynucleotide
  source 1..148: mol_type = other DNA
actcctgggttcaagcaatcctcctgcctcggcctcccaaagtgctgggattataggcgtaagccaccctgtctggcctgtgcaaatgttttaaatgcatgtgtgtgtgactgtgagtgtgaatatatgtgtatgtgtgtgtgtgagt

SEQ ID NO: 201 - DNA, 116 bp, synthetic construct
  misc_feature 1..116: note = Polynucleotide
  source 1..116: mol_type = other DNA
tcctgggttcaagcaatcctcctgcctcggcctcccaaagtgctgggattataggcgtaagccaccctgtctggcctgtgcaaatgttttaaatgcatgtgtgtgtgactgtgagt

SEQ ID NO: 202 - DNA, 76 bp, synthetic construct
  misc_feature 1..76: note = Polynucleotide
  source 1..76: mol_type = other DNA
tcctgggttcaagcaatcctcctgcctcggcctcccaaagtgctgggattataggcgtaagccaccctgtctggcc

SEQ ID NO: 203 - DNA, 56 bp, synthetic construct
  misc_feature 1..56: note = Polynucleotide
  source 1..56: mol_type = other DNA
gggttcaagcaatcctcctgcctcggcctcccaaagtgctgggattataggcgtaa

SEQ ID NO: 204 - DNA, 22 bp, synthetic construct
  misc_feature 1..22: note = Polynucleotide
  source 1..22: mol_type = other DNA
aagcaatcctcctgcctcggcc

SEQ ID NO: 205 - DNA, 18 bp, synthetic construct
  misc_feature 1..18: note = Polynucleotide
  source 1..18: mol_type = other DNA
ctgggattataggcgtaa

SEQ ID NO: 206 - DNA, 21 bp, synthetic construct
  misc_feature 1..21: note = TARDBP Primer 1
  source 1..21: mol_type = other DNA
cagctcatcctcagtcatgtc

SEQ ID NO: 207 - DNA, 21 bp, synthetic construct
  misc_feature 1..21: note = TARDBP Primer 2
  source 1..21: mol_type = other DNA
gatggtgtgactgcaaacttc

SEQ ID NO: 208 - DNA, 18 bp, synthetic construct
  misc_feature 1..18: note = ACTL6B Primer 1
  source 1..18: mol_type = other DNA
tctgagccaaacctgcac

SEQ ID NO: 209 - DNA, 21 bp, synthetic construct
  misc_feature 1..21: note = ACTL6B Primer 2
  source 1..21: mol_type = other DNA
atcagctctgtcagcttctcc

SEQ ID NO: 210 - DNA, 23 bp, synthetic construct
  misc_feature 1..23: note = TARDBP probe
  source 1..23: mol_type = other DNA
cagcgccccacaaacacttttct

SEQ ID NO: 211 - DNA, 21 bp, synthetic construct
  misc_feature 1..21: note = ACTL6B probe
  misc_feature 9..10: note = ZEN quencher between nucleotides 9 and 10
  source 1..21: mol_type = other DNA
cgaggctccgtggaacacacg